SEQUENCE LISTING (29 sequences)

SEQ ID NO: 1 - RNA, 1842 nt, Homo sapiens
  source 1..1842: mol_type = mRNA
ctgctggggagtcgcctacgcctacttcctgccgggaggaggggctcgagttccgcgtcgtcgcgcagagctgactctgggaggcgtttgggcccagagaagtggatccgccgcttgcgccgcatggagtccgaatcggaaagcggggctgctgctgacacccccccactggagaccctaagcttccatggtgatgaagagattatcgaggtggtagaacttgatcccggtccgccggacccagatgacctggcccaggagatggaagatgtggactttgaggaagaagaggaggaagagggcaacgaagagggctgggttctagaaccccaggaaggggtggtcggcagcatggagggccccgacgatagcgaggtcacctttgcattgcactcagcatctgtgttttgtgtgagcctggaccccaagaccaataccttggcagtgaccgggggtgaagatgacaaagccttcgtatggcggctcagcgatggggagctgctctttgagtgtgcaggccataaagactctgtgacttgtgctggtttcagccatgactccactctagtggccacaggggacatgagtggcctcttgaaagtgtggcaggtggacactaaggaggaggtctggtcctttgaagcgggagacctggagtggatggagtggcatcctcgggcacctgtcctgttggcgggcacagctgacggcaacacctggatgtggaaagtcccgaatggtgactgcaagaccttccagggtcccaactgcccagccacctgtggccgagtcctccctgatgggaagagagctgtggtaggctatgaagatgggaccatcaggatttgggacctgaagcagggaagccctatccatgtactgaaagggactgagggtcaccagggcccactcacctgtgttgctgccaaccaggatggcagcttgatcctaactggctctgtggactgccaggccaagctggtcagtgccaccaccggcaaggtggtgggtgtttttagacctgagactgtggcctcccagcccagcctgggagaaggggaggagagtgagtccaactcggtggagtccttgggcttctgcagtgtgatgcccctggcagctgttggctacctggatgggaccttggccatctatgacctggctacgcagactcttaggcatcagtgtcagcaccagtcgggcatcgtgcagctgctgtgggaggcaggcactgccgtggtatatacctgcagcctggatggcatcgtgcgcctctgggacgcccggaccggccgcctgcttactgactaccggggccacacggctgagatcctggactttgccctcagcaaagatgcctccctggtggtgaccacgtcaggagaccacaaagcgaaagtattttgtgtccaaaggcctgaccgttaatggctgcagcccctgcctgtgtgtctggtgttgaggggacgaagggacccctgcccctgtctgccagcagaggcagtagggcacagagggaagaggagggtggggccctggatgactttccagcctcttcaactgacttgctcccctctccttttcttctctttagagacccagcccagggccctcccacccttgtccagacctggtgggcccttcagagggaggggtggacctgtttctctttcactttcatttgctggtgtgagccatggggtgtgtatttgtatgtggggagtaggtgtttgaggttcccgttctttcccttcccaagtctctgggggtggaaaggaggaagagatactagttaaagattttaaaaatgtaaataaaatatacttcccagaaaaaaaaaaa

SEQ ID NO: 2 - RNA, 2175 nt, Homo sapiens
  source 1..2175: mol_type = mRNA
tttcccgccctctagtcgtgcgcggatctgacgcctgacgtaattgcgtagacgccattttagccggtcagacaagcactggacgtggcggccattttgttttggacaccgagcaggagctggcggccgctgcagacgaaaggcaggaaagggcaggccgggtgagcagacggatcggccgactagacagccaaccagcaacaacgaactgagctcgcatactaccgcttacgcatctaaccaaccgcccatctagctaacccgagcccctccaccgtcaactcaggttcggccggtccccggcccgcctgccggagccgtggtggcagccccgggaggagcactggcgtctgtttccttcgattctcgggattcgaagatggctgcacagtcagcgccgaaagttgtgctaaaaagcaccaccaagatgtctctaaatgagcgctttactaatatgctgaagaacaaacagccgacgccagtgaatattcgggcttcgatgcagcaacaacagcagctagccagtgccagaaacagaagactggcccagcagatggagaatagaccctctgtccaggcagcattaaaacttaagcagaagagcttaaagcagcgcctgggtaagagtaacatccaggcacggttaggccgacccataggggccctggccaggggagcaatcggaggacgaggcctacccataatccagagaggcttgcccagaggaggactacgtgggggacgtgccaccagaaccctacttaggggcgggatgtcactccgaggtcaaaacctgctccgaggtggacgagccgtagctccccgaatgggcttaagaagaggtggtgttcgaggtcgtggaggtcctgggagagggggcctagggcgtggagctatgggtcgtggcggaatcggtggtagaggtcggggtatgataggtcggggaagagggggctttggaggccgaggccgaggccgtggacgagggagaggtgcccttgctcgccctgtattgaccaaggagcagctggacaaccaattggatgcatatatgtcgaaaacaaaaggacacctggatgctgagttggatgcctacatggcgcagacagatcccgaaaccaatgattgaagcctgcccatcctcccatgagagactcttgttagtcaacacatctgtaaataaccttgagataacagatgagaagaaatctgattgatgctggatggacctatcacaataggctgtggacttacttgccaccagcttgtgcatttagtgtgttccttttactttttgatactgtgttgtatgaaacccttttgtcctttgatttggttttttgtttttgtttttttaggggggagggggggtttcccctcctttgcccagacttctctttgaacacaaatgcattagccttgtggctagaacaccctcttcctacctctgtctcccctcacttgtcatatgctctgacatgctaacatttcttttgttcatccctgttgcccccacagaaacatcccagaaaaaccggtcagtgttccttcctccctgatccttaggtttctgaaatagggttctgttacatcctcttcgatagcctgtttaaaatgtttagaaggtctggagctcaaaaatgcgttcttccacattgataatttagtaaactgagaacattgacatcactacagggcagcataagaggttgcttacatgtggtagcagctctggtttgattcaagttgctaccatgtacattgacagcacatataccataaccagcgtgttgggttgaattgcactttctacctttgtatgagatttacagactttccttctgggtttgtatcatgaccagaggggtactatagggttggtttatactgcaatatagaggatcagaagccatttgatttggtaggtgtgtcagaagggagaatgatggcagacgaactgctggaagaggtcagaagatagccatgctaaaatgcaattatatcctcatgtttatcccaaactaatcttggacttttccactcattagctttgttttgcccttgtttcccttgaaggtttaagttcaaccatattctgtcaactgttcagtttcagtggaatcttgtatttctggttcattataacaaactgttcgcttaaatccaaaaaaaaaa

SEQ ID NO: 3 - RNA, 4811 nt, Homo sapiens
  source 1..4811: mol_type = mRNA
tttcttctccggccctggcaggttccgggagcctcggctcgtgggtgccggaagtggaggcggttggtggggttggcggggctcagcgacgctgcgcgggtggcggtttgcgaactgcgggtggactgtgtagtgaccggcgtcccgctgtctcgccccgtggcgggtgagcgagggtgcgtggtgcgcggcggcggcggaacgaacgcggtgcgggcggggcgcccgccgcagggcccatggcctcctgcgcgagcatcgacatcgaggacgccacgcagcacctgcgggacatcctcaagctggaccggcccgcgggcggccccagtgcagagagcccacggccatccagtgcctacaatggggacctcaatggacttctggtcccagacccgctctgctcaggtgatagtacctcagcaaacaagactggtcttcggaccatgccacccattaacctgcaagagaagcaggtcatctgtctctcaggagatgatagctccacctgcattgggattttggccaaggaggtggagattgtggctagcagtgactctagcatttcaagcaaggcccggggaagcaacaaggtgaaaattcagcctgtcgccaagtatgactgggaacagaagtactactatggcaacctgattgctgtgtctaactccttcttggcctatgccattcgggctgccaacaatggctctgccatggtgcgggtgatcagcgtcagcacttcggagcggaccttgctcaagggcttcacaggcagtgtggctgatctggctttcgcgcacctcaactctccacagctggcctgcctggatgaggcaggcaacctgttcgtgtggcgcttggctctggttaatggcaaaattcaagaagagatcttggtccatattcggcagccagagggcacgccactgaaccactttcgcaggatcatctggtgccccttcatccctgaggagagcgaagactgctgtgaggagagcagcccaacagtggccctgctgcatgaagaccgggctgaggtgtgggacctggacatgctccgctccagccacagtacctggcctgtggatgttagccagatcaagcagggcttcattgtggtaaaaggtcatagcacgtgcctcagtgaaggagccctctctcctgatgggactgtgctggctactgcgagccacgatggctatgtcaagttctggcagatctacattgaggggcaagatgagccaaggtgtctgcacgagtggaaacctcatgatgggcggcccctctcctgcctcctgttctgtgacaaccataagaaacaagaccctgatgtccctttctggaggttccttattactggtgctgaccagaaccgagagttaaagatgtggtgtacagtatcctggacctgcctgcagactattcgcttctccccagatatcttcagctcagtgagtgtgccccctagcctcaaggtttgcttggacctctcagcagaatacctgattctcagcgatgtgcaacggaaggtcctctatgtgatggagctgctgcaaaaccaggaggagggccacgcctgcttcagctccatctcggagttcctgctcacccaccctgtgctgagctttggtatccaggttgtgagtcgctgccggctacggcacactgaggtgctgcctgccgaagaggaaaatgacagcctgggtgctgatggtacccatggagccggtgccatggagtctgcggccggtgtgctcatcaagctcttttgtgtgcatactaaggcactgcaagatgtgcagatccgcttccagccacagctgaaccctgatgtggtggccccactgcccacccacactgcccacgaggacttcacatttggagagtctcggcccgaactgggctctgagggcctggggtcagccgctcacggctcccagcctgacctccgacgaatcgtggagctgcctgcacctgccgacttcctcagtctgagcagtgagaccaagcccaagttgatgacacctgacgccttcatgacacctagcgcctccttgcagcagatcactgcctctcccagcagcagcagcagcggtagcagcagcagcagcagcagtagcagcagctcccttacagctgtgtctgccatgagcagcacctcagctgtggacccctccttgaccaggccacctgaggagctgaccttgagccccaagctgcagctggatggcagcctgacaatgagcagcagtggcagccttcaggcaagcccgcgtggcctcctgcctggcctgctcccagccccagctgacaaactgactcccaaggggccgggccaggtgcctactgccacctctgcactgtccctggagctgcaggaagtggagcccctggggctaccccaagcctcccctagccgcactcgttcccctgatgtcatctcctcagcttccactgccctgtcccaggacatccctgagattgcatctgaggccctgtcccgtggttttggctcctctgcaccagagggccttgagccagacagtatggcttcagccgcctcggcactgcacctgctgtccccacggccccggccagggcccgagctcggcccccagctcgggcttgatggaggccctggggatggagatcggcataataccccctccctcctggaggcagccttgacccaggaggcctcgactcctgacagtcaggtttggcccacagcacctgacattactcgtgagacctgcagcaccctggcagaaagccccaggaatggccttcaggaaaagcacaagagcctggccttccaccgaccaccatatcacctgctgcagcaacgtgacagccaggatgccagtgctgagcaaagtgaccatgatgatgaggtggccagccttgcctctgcttcaggaggctttggcaccaaagttcctgctccacggctgcctgccaaggactggaagaccaagggatcccctcgaacctcacccaagctcaagaggaaaagcaagaaggatgatggggatgcagccatgggatcccggctcacagagcaccaggtggcagagccccctgaggactggccagcactaatttggcaacagcagagagagctggcagagctgcggcacagccaggaagagctgctgcagcgtctgtgtacccaactcgaaggcctgcagagcacagtcacaggccacgtagaacgtgcccttgagactcggcacgagcaggaacagcggcggctggagcgagcactggctgaggggcagcagcggggagggcagctgcaggagcagctgacacaacagttgtcccaagcactgtcgtcagctgtagctgggcggctagagcgcagcatacgggatgagatcaagaagacagtccctccatgtgtctcaaggagtctggagcctatggcaggccaactgagcaactcagtggctaccaagctcacagctgtggagggcagcatgaaagagaacatctccaagctgctcaagtccaagaacttgactgatgccatcgcccgagcagctgcagacacattacaagggccgatgcaggctgcctaccgggaagccttccagagtgtggtgctgccggcctttgagaagagctgccaggccatgttccagcaaatcaatgatagcttccggctggggacacaggaatacttgcagcagctagaaagccacatgaagagccggaaggcacgggaacaggaggccagggagcctgtgctagcccagctgcggggcctggtcagcacactgcagagtgccactgagcagatggcagccaccgtggccggcagtgttcgtgctgaggtgcagcaccagctgcatgtggctgtgggcagcctgcaggagtccattttagcacaggtacagcgcatcgttaagggtgaggtgagtgtggcgctcaaggagcagcaggccgccgtcacctccagcatcatgcaggccatgcgctcagctgctggcacacctgtcccctctgcccaccttgactgccaggcccagcaagcccatatcctgcagctgctgcagcagggccacctcaatcaggccttccagcaggcgctgacagctgctgacctgaacctggtgctgtatgtgtgtgaaactgtggacccagcccaggtttttgggcagccaccctgcccgctctcccagcctgtgctcctttccctcatccagcagctggcatctgaccttggcactcgaactgacctcaagctcagctacctggaagaggccgtgatgcacctggaccacagtgaccccatcactcgggaccacatgggctccgttatggcccaggtgcgccaaaagctttttcagttcctgcaggctgagccacacaactcacttggcaaagcagctcggcgtctcagcctcatgctgcatggcctcgtgacccccagcctcccttagctgctaagcctgccttgcccaggggtgggatggcactgaaggccagcagacaggcctaggctggggcagggtcacggctggcctttacctgctcaggcccccatctctggggtgtttgggggtcagggagcagggagcactggccgtggtctacagcgtgtggtagtcagaaggtttagctgggcccagggcaggtattgcgcctgcttgggttctgccatgcctggagcatgaccctgagatcgtgacaccacttgagtggaattttccatgttcctttttacctctaatttggatctttttgtttttgaaaaacattgagaaattcaattaaatgcttttggaataaaatggagtatgtgtgtg

SEQ ID NO: 4 - RNA, 8518 nt, Homo sapiens
  source 1..8518: mol_type = mRNA
accctcttggtgtgcgcgtcatggccgtcagcaccgcgttcccgtcctcttccgcttggccccagaaagtttcggttctgcccggcggtggacccacgagcgcgtgccaccatggagtctgaccactgctgagcagacagccaccgagggccgaaattctgagccttcctctggacccaggcaggagacatacagacaagaaaggcaaactcaccatggcctccaccaatgcagagagccagctccagagaatcatccgagacttgcaagatgctgtgacagaactaagcaaagaatttcaggaagcaggggaacccatcacggatgacagcaccagcttgcataaattttcttataaacttgagtatctcctgcaatttgatcagaaagagaaggccaccctcctgggcaacaagaaggactactgggattacttctgtgcctgcctggccaaggtgaaaggagccaatgatgggatccgctttgtcaagtctatctcagagctccgaacatccttggggaaaggaagagcatttattcgctactccttggtgcaccagaggttggcagacaccttacagcagtgcttcatgaacaccaaagtgaccagtgactggtactatgcaagaagcccctttctgcagccaaagctgagctcggacattgtgggccaactctatgagctgactgaggttcagtttgacctggcgtcgaggggctttgacttggatgctgcctggccaacatttgccaggaggacgctgaccactggctcttctgcttacctgtggaaaccccctagccgcagctccagcatgagcagcttggtgagcagctacctgcagactcaagagatggtgtccaactttgacctgaacagccccctaaacaacgaggcattggagggctttgatgagatgcgactagagctggaccagttggaggtgcgggagaagcagctacgggagcgcatgcagcagctggacagagagaaccaggagctgagggcagctgtcagccagcaaggggagcaactgcagacagagagggagagggggcgcactgcagcggaggacaacgttcgcctcacttgcttggtagctgagctccagaagcagtgggaggtcacccaggccacccagaacactgtgaaggagctgcagacatgcctgcagggcctggagctaggagcagcagagaaggaggaggactaccacacagccctgcggcggctggagtccatgctgcagcccttggcacaggagcttgaggccacacgggactcactggacaagaaaaaccagcatttagccagcttcccaggctggctagccatggctcagcagaaggcagatacggcatcagacacaaagggccggcaagaacctattcccagtgatgcggcccaggagatgcaggagctaggggagaagcttcaagccctagaaagggagagaaccaaggtcgaggaggtcaacagacagcagagtgcccaactggaacagctggtcaaggagcttcagctgaaagaggatgcccgggccagcctggagcgcctggtgaaggagatggccccactccaggaggagttgtctgggaagggacaggaggcagaccagctctggcgacggctgcaggagttgctggcccacacgagctcctgggaggaggagctagcagagttgaggcgggagaaaaaacagcaacaggaggagaaggagctgctggagcaggaggtcaggtctctgacccggcagctgcagttcctggagacccagctggcacaggtgagccaacatgtgagtgacctggaggagcagaagaagcagctcattcaggacaaagaccacctcagccagcaggtgggtatgctcgagcggcttgctgggccgcctggcccagaactgccagtggcaggtgagaagaatgaggccctggtccctgtgaactccagtctgcaagaggcctgggggaagccagaggaggagcagaggggcctgcaggaggcacagttagacgataccaaggtgcaagagggcagccaggaggaagagctccggcaggccaacagggagctggagaaggagctacagaatgtggtcgggcgtaaccagctcctggagggcaagctgcaagccctgcaggccgattaccaggctttgcagcagcgggaatcagccatccagggctccttggcctccctggaggccgagcaggccagcatccggcacttgggtgaccagatggaggcgagcttgctggctgtaaggaaggccaaggaggccatgaaagcccagatggcagagaaggaggccattctacagagcaaggagggcgagtgtcagcagctgcgggaggaggtggagcagtgccagcaactggcagaagcccggcacagagagcttagggctctcgagagccagtgccagcagcagacccagctgattgaggtcctcacagcagagaaaggccaacagggagttggcccacccactgacaatgaagcccgtgagctggctgcccagctagccctgtctcaggcgcagctggaagtccatcagggggaggtccaacggctgcaggctcaggtggtggacctccaggccaagatgcgggcagccctggatgaccaggacaaggtgcagagccagctaagcatggctgaggccgtcctgagggagcacaaaacccttgtgcagcagctgaaggagcagaatgaagcccttaacagagcccatgtccaggagctgctgcaatgctcggagcgtgaaggggcactgcaggaggagagggccgatgaggcccagcagagggaggaggagctgcgggccctgcaggaggagctgtcccaggccaaatgcagctccgaggaagcacagctggagcacgctgagctgcaagagcagctgcaccgggccaacacagacacagctgagctgggcatccaggtttgcgcactgaccgtggaaaaggagcgagtggaggaggcactggcctgtgctgtccaggagctccaggacgccaaagaggcagcctcaagggagcgagagggcctggagcgccaagtagctgggctgcagcaagagaaggagagcttgcaggagaagctgaaggcggccaaggcagcagccggctcactgcctggcctgcaggcccagctcgcccaggcagagcagcgggcccagagcctccaagaggctgcacaccaggagctcaacaccctcaagttccagctgagtgctgaaatcatggactaccagagcagacttaagaatgctggtgaagagtgcaagagcctcaggggccagcttgaggagcaaggccggcagctgcaggctgctgaggaagctgtggagaagctgaaggccacccaagcagacatgggagagaagctgagctgcactagcaaccatcttgcagagtgccaggcggccatgctgaggaaggacaaggagggggctgccctgcgtgaagacctagaaaggacccagaaggaactcgaaaaagccacaacaaaaatccaagagtattacaacaaactctgccaggaggtgacaaatcgtgagaggaatgaccagaagatgcttgctgacctggatgacctcaacagaaccaagaagtatctcgaggagcggctgatagagctgctcagggacaaggatgctctctggcagaagtcagatgccctggaattccagcagaagctcagtgctgaggagagatggctcggagacacagaggcaaaccactgcctcgactgtaagcgggagttcagctggatggtgcggcggcaccactgcaggatatgtggccgcatcttctgttactactgctgcaacaactacgtcctgagcaagcacggtggcaaaaaggagcgctgctgccgagcctgtttccagaagctcagtgaaggccctggctcccctgatagcagtggctcaggcactagccagggagagcccagccctgcactgtcaccagcctcacctgggccccaggccacaggaggccaaggagcaaatacagactacaggccaccggacgacgctgtgtttgatatcatcacagatgaggaattgtgccagatacaggagtccggctcctctttgcctgaaacacccactgaaactgattctctcgacccaaatgcggctgaacaggatactacatcaacctcgctaacgcctgaggacactgaagacatgcccgtggggcaggattcggaaatctgcctgctgaagtctggagaactgatgatcaaagtacccctcacagtggatgagatcgccagcttcggggagggtagcagggagctgtttgtgaggtccagcacctacagcctgatccccatcactgtggccgaggcaggcctcaccatcagctgggtcttctcctctgaccccaagagcatctccttcagtgtggtcttccaggaggccgaggacacaccgctggatcagtgtaaggtcctcattcccacgacccgatgcaactcccacaaggagaacatccagggccagctcaaggttcgcacacccggcatctacatgctcatcttcgacaataccttctcaaggtttgtctctaaaaaggtattttatcacttgacggttgatcggcctgtgatctacgatggaagtgatttcctgtagcttcagcacctcagtaacttcacttcatccacaggaaacactgctcttcctcacctgtcacataaagcatttttttaaaaagtcagctgctccaaaatcatcaactcagcccctgggctgcccctcagaggcggtgtctggggaggactttgtgctcagcactctgcaccggccactcttagcccccgaggcgttgaagggctcaggcaatgtttccattaagtagagactcagctgttgtcacacccaaagggatgctctgccaaaggtttaaacacccaggagaccatcagcctctcctgggagcacagttggctacaggcctcttgtggagagtttcacgggcaggggtgattccaacttctgcctgtggagagattttctgccctgccccaccagggccctgcatgttggagactgagctgggtgcactggccatgccctgtgaatcctcaggctgtgacgccctcaggtactcctgggaaaaggaggtacacagccatcatgcgagtcggtgccaggggaccccccggagatcctgaccagctcctccagtcatgctcttgtccctcactgccccagtaagctggaggctgctccagaactcagcagtgttggaggggcctctaagctgcactctctttctggcccttttgtctgggtgattctgtcctcaaataaagcccttcactcagccagacctctccacagctcaaagcattgccctaagaatcagaagtaaagataatccaagagcaaaacccactgtacttggggcctgcaatggctgtgtgtacactacatctaatgcccaaatgccagccagtgtggatgttgtgaccacagagcaggattgtgcattggctttagagctactcctcagctgatggcccacttttgtttatataaataagagcttctgccccacctgcagacatgtttactaatgatcatagccaggattagaaccactttcaaacattggggccttcttaacaaaagtctttgataacttaagaaccaaagtaacagagtaaacagaggcatgatggatccctgggccccactcccctcctgacaggttccccaacagcccatttgcccacttcccactgctcagcccacaccagacctccaggagacatccccccttgaggcagagagatcctgttccctattcccagacaagaattatttaatcttccctgttctctgtggtccttttcttccccaacaacagatagctcaccttggacagctcttcgtcccttgttcatggaaccagctgcctgcagtcaggccccaggttcttccatgggtgaacagagcatctgacaaaaggtcccagtttggccaggggtgagggagagagcaccagacaggctatccgagaatctgagagctgggcccggcagttcctccagctacccttgtgacctaagtccagtcacacatttcccaaagtttctctttgtcataaccctggtctggctggttttgagggcttgagaatgggtcagggactccaggccaagtccaacagagaccccaaacccaccacacaccagcagccacaacctcaccaccaacaaagaggacttttgtggggccacaagtaagaggtcatttctggaatggactcagacctttaaacaggagagttgagcacttccagtcagtttttaagcaaggcatggggaacagggaatagaacctttcaaagaggttgcccagagaaaagctgggcctcttgcattcggcttccttggagcagcctcttctggcagaaagccatcaggtgctcaatcatcttctcctggccaaggctctgaccatgcttagtactggaatagaggtggccaggcccccagcgactcttcttggcctgatgtttgtcctcacaggcatgccacgtggcctgagatgattcagaacaaatcatgctaactttgaatccatccagccacttgcaaatgataatcagaagtcagcttgttcactgttagaaagaaactaacaaaagagaacccagagcaatctagaatctttgagtgcttggctttccaaggatactgcggagactctggccaagctgatgaccttctgaagtgtcactggcaccatatgcaacaagaaccaccattcactgagtagctaatgggtttggggcctgggacattccatctgaggtccttcctgaacatgtcactccacagcagaggaccggttgcagcttacccagaaccactcctccaggagagctggatgttttgcgtgcaacaccttgagcactgactgctattgttcaaaaaaagcctttgctgcattcggaggactgccccgtgccctgaggtgacttcctaactatgtggtttcattagcgaatttattttttgtgctgggtggacatttgtattttgttaggttgctgtttaagctcaagtttgctgtgctctctgcagctacaaaacatcttggcatatttaagagtggcttttataaatagctttattctgatattaatcagattcccaactttactgagaattaaggactggggtactttaaagaaatgcaaatagcaattgaagaaccactgctgcaggtggtagccctggctagactgaattacactagaaatcagccagaaggaagcgtccttgggatcccagatcactctttttttttttttttaaaaggggcagccccttgatggctcatctctctgaataacagttacgtcttcatatcgataccagatgccttcttcatcatgccactgaagccactcaccaccttcaagaacatgccaacctctgtcagattcacttacccacaaacaaggaggcacgtttggcacaaagtgttgtcctccaggtccaagtggactctacagagtgcttgacctcaacacactggattccaggtggactggaccaagagcaggcaaagacacgggaactgaaaaactccacagggtttggagaatagaaatgaaaagccacgtcatataactcaagaataaatggtgttttggaaattttaaaattatcatcgaaggtggtgaaactatttcaggcccaaatgaaaggaaatcgccagttggggatgaaatcacagagcctgtgttttatgatatggttggatgtccactgatgaaattttaaaggagtttcagttttaaaagtgcgcatgattctacatatgagaattctttaggccaagaaactgtccttggctcagaggtgttgggaattaaagcagagagaagccattcgtgatgcttagaaccaaggatggtcatgtacacaaagaccatcgagacggccattcttgtttacaaaacacttaccaagaaagcactttgtaggggaactttagtaagttcttctcatttcattatgtttcttccaaggaaacaggagagactgaattaataattctctctttcctcttaagcacttttaaaataataaagtacatcttgaaatttggggaggcatctctgatttaaaaaaagaaaaaggctgcttgatgtatgttatgcagagacactctgcctctggtggctgcagagcaatacccaagcctcatttggaaggctcaacatttggaattgcactttaattgattaatcctcaattcatgtggccttacgggatggtgggtctgggaccccaattcattcttatctgccaaagaattatctagaagcacatcaaataccagcaccccacctgctcaatgggggtggaaaacttttgtatccctaagcatattattttatagtgtctgccatgccatgtggaaatactttatttttaacctcaggatttaaataaagtaaacactatgacatttagaca

SEQ ID NO: 5 - RNA, 6789 nt, Homo sapiens
  source 1..6789: mol_type = mRNA
ctcgcgccaggcgagtctccgcgtctccctcgcgaactcggtgaaaggaattggcgccgttcgacaccaggcggatccgctctgcagcacgaacccatctccagccgcagccgcagccgccgcccgggccgaggagcagccgcagcagccgccaccagtggccgagtgagcggagccgagtttgaggcagcgcctagcggtgaatcggggccctcaccatgagttcctcgcctgttaatgtaaaaaagctgaaggtgtcggagctgaaagaggagctcaagaagcgacgcctttctgacaagggtctcaaggccgagctcatggagcgactccaggctgcgctggacgacgaggaggccgggggccgccccgccatggagcccgggaacggcagcctagacctgggcggggattccgctgggcgctcgggagcaggcctcgagcaggaggccgcggccggcggcgatgaagaggaggaggaagaggaagaggaggaggaaggaatctccgctctggacggcgaccagatggagctaggagaggagaacggggccgcgggggcggccgactcgggcccgatggaggaggaggaggccgcctcggaagacgagaacggcgacgatcagggtttccaggaaggggaagatgagctcggggacgaagaggaaggcgcgggcgacgagaacgggcacggggagcagcagcctcaaccgccggcgacgcagcagcaacagccccaacagcagcgcggggccgccaaggaggccgcggggaagagcagcggccccacctcgctgttcgcggtgacggtggcgccgcccggggcgaggcagggccagcagcaggcgggaggggacggcaaaacagaacagaaaggcggagataaaaagaggggtgttaaaagaccacgagaagatcatggccgtggatattttgagtacattgaagagaacaagtatagcagagccaaatctcctcagccacctgttgaagaagaagatgaacacttcgatgacacagtggtttgtcttgatacttataattgtgatctacattttaaaatatcaagagatcgtctcagtgcttcttcccttacaatggagagttttgcttttctttgggctggaggaagagcatcctatggtgtgtcaaaaggcaaagtgtgttttgagatgaaggttacagagaagatcccagtaaggcatttatatacaaaagatattgacatacatgaagttcgtattggctggtcactaactacaagtggaatgttacttggtgaagaagaattttcttatgggtattctctaaaaggaataaaaacatgcaactgtgagactgaagattatggagaaaagtttgatgaaaatgatgtgattacatgttttgctaactttgaaagtgatgaagtagaactctcgtatgctaagaatggacaagatcttggcgttgccttcaaaatcagtaaggaagttcttgctggacggccactgttcccgcatgttctctgccacaactgtgcagttgaatttaattttggtcagaaggaaaagccatattttccaatacctgaagagtatactttcatccagaacgtccccttagaggatcgagttagaggaccaaaggggcctgaagagaagaaagattgtgaagttgtgatgatgattggcttgccaggagctggaaaaactacctgggttactaaacatgcagcagaaaatccagggaaatataacattcttggcacaaatactattatggataagatgatggtggcaggttttaagaagcaaatggcagatactggaaaactgaacacactgttgcagagagccccccagtgtcttgggaaatttattgagattgctgcccgaaagaagcgaaattttattctggatcagacaaatgtgtctgctgctgcccagaggagaaaaatgtgcctgtttgcaggcttccagcgaaaagctgttgtagtttgcccaaaagatgaagactataagcaaagaacacagaagaaagcagaagtagaggggaaagacctaccagaacatgcggtcctcaaaatgaaaggaaactttaccctcccagaggtagctgagtgctttgatgaaataacctatgttgaacttcagaaggaagaagcccaaaaactcttggagcaatataaggaagaaagcaaaaaggctcttccaccagaaaagaaacagaacactggctcaaagaaaagcaataaaaataagagtggcaagaaccagtttaacagaggtggtggccatagaggacgtggaggattcaatatgcgtggtggaaatttcagaggaggagcccctgggaatcgtggcggatataataggaggggcaacatgccacagagaggtggtggcggtggaggaagtggtggaatcggctatccataccctcgtgcccctgtttttcctggccgtggtagttactcaaacagagggaactacaacagaggtggaatgcccaacagagggaactacaaccagaacttcagaggacgaggaaacaatcgtggctacaaaaatcaatctcagggctacaaccagtggcagcagggtcaattctggggtcagaagccatggagtcagcattatcaccaaggatattattgaatacccaaataaaacgaactgatacatatttctccaaaaccttcacaagaagtcgactgttttctttagtaggctaactttttaaacattccacaagaggaagtgcctgcgggttccttttttagaagctttgtgggttgattttttttcttttcttttttgtacatttttaattgcagtttaaaagtgaatcgtaagagaacctcagcattgtgcacgataagagaatgtgtcagtatttcagggttctacattttatctgtaaaatgtgacttttttttttttttatcacaacagaagtaaaatgttgctttgtacctggtgtcttttattaagaatttactccccccatttctcacagagaataacagtcgggagtcattgtcacaatataatagaaatgttagcaaccagattcatgtaaggactaagtggtcctcatgaattgcattaagactctgtactgctcatattacactccatcctctctgtagtttgctgggtagtggagggggtaagctaaatcatagtttctgacaataactgggaaggttttttcttaaaataacaatggaattggtataattgggattgaaaactaaaacttggaactaagatagagaagatggagtgtatgtagaagggctgttaaaaatgtaaaacttggttgcattatttgtggaggctcaaacttgtgaaggttaataccataatttttccatttgttctgcattttgattctgaaaagaaagctggctttgcccatttcttattaaaaaaacttgttgtaaatccagttgtctaatgggatctatatgaagttagccatgtctgtatgcccttctcccacaaaatactgtataactagtgtgcttgtagtagttaactccaccatctttgtaagctaatgaaattgtgagtcacccatttatatcttaatttttaatcatgtcagttcttgaatgggtatctccttagcctgctgatttctttttctttctaaagaaagtgggtggagaaattaatttagacgtttgtttgcaataaaaagaattcattttactcttgttttgggattctcgccatcaaggttcaaaatccctttatataactcccaagaggagaaatttattaagtgtgtgctttctggacagcttattctttactctgcatagaacatttaggttttaaaaacttaaatgtatactgacaattgatacataattatgaagtaaagttgaattcttcccttcccctcccccccagacaacttttaacatatttaatgaggggaaaaggtactggctgggagaagttaacactgagtttatcatctttacagaatgctaatgctgtcctcaactgattattttatatacatatatatgatacatgaaactctgggatcagatgcttttagaagccatcatgcaagccagtcattgatgtcactgctacacaacactgctaacttgactgtagctatgtaataacattagatcccctaattgtaattatattgggtttgcacagaacactttaatcttcccctcaccaatgtgaagtgaggaatcaggagtcaaactgtagaactaaaatttgacttcagtctagcgtttccttggtgtttttaggttgctttggtaagtttaggtttgctatatttctgattgcttagaattttgttttagccctttaaaatcagatcataaatatgaattcatacttctaaggaattttcttgctataagctggagtttaggtgatgtataggttcagttgagacatttttggaacaggcaaatccttagttaacataagatatttaacagttgaagatagtgtcatggatttttatcttttttagcaagtaatgctaagaaccactggcctgagctactactcttcagtatacattattaggattgcatagacttactagaggaacagtttcaggttttgatgctaatcagtgttgtgtcctaaagttgtcctttgtgcctttaaaaagttttggatatatcttctagtttaaaattgcttattaaggaattcattttataattgcagtgggaaagtaatggtcaagtaacactaggtagactatcatgcctgtttagcccagagaatttggggggagagagaatagataaaaatggcacccagaaaaatgttaaaatctttagtcaagactagaattaatacaattgtctacacttgtatggcagaaataaccttataaagtgtttaaggaattcagagaagggaatgtaccaaataagcaacagggagaaaattaggtaagaagtaagatacgaacgagaaacctgatttattgctcatccttcccttgcctccctaatggcaagcaaaactctgaacatctgaaaaggatgtagttctggacaaatcctgactacccagaggaaactcactgtgagattgctgttgatttgaagggtgctttcactaaggttatattttaaagtagaataacacatgctgagtgtaaactggctttggattggtcagctgcagtagtacaaaaacagcatagaatttgagaaaactaaaactgctatgagatagctatgagaaaactaaaactgctatgagatagaaatgatgtaaaattatgtggaaagttttccctcatatactcacatacagcctttgaagggctctggctctgaccggttgatggccttgagcgagatgaaatcatgaaattgagtcaaatcaatttgacattgaaatgacaagaggaaactcttaaatacataaaaacaagctctcatttgcctaggatagatactgtcttaaaaataaagactgaacctagatgttctgagcactagcaacaaggtattttaacaagtttaaaggaattctctgaaaaagttataaaattattctaggaaacataaccataatagtgttttaagggactttcacctggggattttatattcatgaacagagtgtattctgtatttaaaatgtctcatttgtgggaattggatgacatgttttttgataaatttattcacaatataaattgactttttattctaggaccatgtgaataatgggttccattgcacaaatacaaatattttaatagcttcttaggcagtggtgtagacatcttggatataaataattgtagatcttgtatatttgatttttaaaaaactagaataaacagagaggcataaacatatcttagagtccaagtggtagtgtttagcattggatataataaatggatgttttacaaagtgtttccataattctcttcctatacataaatgtcttgttttcaaaagtggatggaacttggctgggtgtggtggctcacgcctgtaatcctagcactttgggaggccaaggcgggaggatcacttgagctcaggagtttgagaccatcctgggcaacatagtgagacctggtctcttgaaaaaaaaaaagtggatggaacttgtagcagagaattttatctacttctcaacctgcttcagaatacccatttgagatgttcccctggaaagatgaacacaatactgcatctgaagccatttcttcccacctaacattcttaaaatgattagagtctaaactttgtcattcattcctaattctggagctctgaggttgaggtgttcagagtttggtgaataattgggtttaagttttaacattttagtaataataaaagcaaacacatacatgttaaggcctgacaataggttgcaatacccatgcattgggactcatacccagcataaatggtgagggactgaacattagtgctttgagcaagaattggttaactacctgagatctctaaaacagtaatttgaattactaatacataaaccacaagtctcttcgaattgttaattgcctaaatttaccctaaaacttagttaacgctgcagtgcctttattaaagcttttttttgggggggggtgtgggcacagctactccataactttaaattttaaagcatgaaggtgatctagctagtgttagtgcttgagttggtagttacataggctgctcttgaatcgtcctgttttctttgcctagtattagcaccacaggtacatatttgtaaaataagcattaaaagtactttgtccagc

SEQ ID NO: 6 - RNA, 1809 nt, Homo sapiens
  source 1..1809: mol_type = mRNA
tcgagcccgctttccagggaccctacctgagggcccacaggtgaggcagcctggcctagcaggccccacgccaccgcctctgcctccaggccgcccgctgctgcggggccaccatgctcctgcccaggcctggagactgacccgaccccggcactacctcgaggctccgcccccacctgctggaccccagggtcccaccctggcccaggaggtcagccagggaatcattaacaagaggcagtgacatggcgcagaaggagggtggccggactgtgccatgctgctccagacccaaggtggcagctctcactgcggggaccctgctacttctgacagccatcggggcggcatcctgggccattgtggctgttctcctcaggagtgaccaggagccgctgtacccagtgcaggtcagctctgcggacgctcggctcatggtctttgacaagacggaagggacgtggcggctgctgtgctcctcgcgctccaacgccagggtagccggactcagctgcgaggagatgggcttcctcagggcactgacccactccgagctggacgtgcgaacggcgggcgccaatggcacgtcgggcttcttctgtgtggacgaggggaggctgccccacacccagaggctgctggaggtcatctccgtgtgtgattgccccagaggccgtttcttggccgccatctgccaagactgtggccgcaggaagctgcccgtggaccgcatcgtgggaggccgggacaccagcttgggccggtggccgtggcaagtcagccttcgctatgatggagcacacctctgtgggggatccctgctctccggggactgggtgctgacagccgcccactgcttcccggagcggaaccgggtcctgtcccgatggcgagtgtttgccggtgccgtggcccaggcctctccccacggtctgcagctgggggtgcaggctgtggtctaccacgggggctatcttccctttcgggaccccaacagcgaggagaacagcaacgatattgccctggtccacctctccagtcccctgcccctcacagaatacatccagcctgtgtgcctcccagctgccggccaggccctggtggatggcaagatctgtaccgtgacgggctggggcaacacgcagtactatggccaacaggccggggtactccaggaggctcgagtccccataatcagcaatgatgtctgcaatggcgctgacttctatggaaaccagatcaagcccaagatgttctgtgctggctaccccgagggtggcattgatgcctgccagggcgacagcggtggtccctttgtgtgtgaggacagcatctctcggacgccacgttggcggctgtgtggcattgtgagttggggcactggctgtgccctggcccagaagccaggcgtctacaccaaagtcagtgacttccgggagtggatcttccaggccataaagactcactccgaagccagcggcatggtgacccagctctgaccggtggcttctcgctgcgcagcctccagggcccgaggtgatcccggtggtgggatccacgctgggcctaggatgggacgtttttcttcttgggcccggtccacaggtccaaggacaccctccctccagggtcctctcttccacagtggcgggcccactcagccccgagaccacccaacctcaccctcctgacccccatgtaaatattgttctgctgtctgggactcctgtctaggtgcccctgatgacgggatgctctttaaataataaagatggttttgattaaaaaaaaaaaaaaaaaaaaaaaaaa

SEQ ID NO: 7 - RNA, 1700 nt, Homo sapiens
  source 1..1700: mol_type = mRNA
aactagcacgtggctcccgcccccagactgcttctttattcattcctaacctttaccttggcagatgaaatataagattcatcaaccacatttgacagcccatggcaggtttcctgttttccatcgtccctctgcaggtcacagacacacagagcccagccgtggcaggctcagccggggtccggggctgctaacaacggctacattcctcccccagggccaagggaaatcctgagcgcaggccagggttgtttggttttgaggtgtgctgggatgaaaggcaccctggaagtggaaggttcggtcattcattaattaattacatctataattgagggtttgttcttaagagcgagtcctttgaaagtactttccttcaaacagtgactgccacaaaggcatcagatattcaccaccttctcggctgcctcagcacagcaagctttattctgggacctgagatcctgttctgagctggctttcccttctccaggctcgctcaccctccctttagagatagtggatggtaagatgaccaatgctcagattattcttctcattgacaatgccaggatggcagtggatgacttcaacctcaagtatgaaaatgaacactcctttaagaaagacttggaaattgaagtcgagggcctccgaaggaccttagacaacctgaccattgtcacaacagacctagaacaggaggtggaaggaatgaggaaagagctcattctcatgaagaagcaccatgagcaggaaatggagaagcatcatgtgccaagtgacttcaatgtcaatgtgaaggtggatacaggtcccagggaagatctgattaaggtcctggaggatatgagacaagaatatgagcttataataaagaagaagcatcgagacttggacacttggtataaagaacagtctgcagccatgtcccaggaggcagccagtccagccactgtgcagagcagacaaggtgacatccacgaactgaagcgcacattccaggccctggagattgacctgcagacacagtacagcacgaaatctgctttggaaaacatgttatccgagacccagtctcggtactcctgcaagctccaggacatgcaagagatcatctcccactatgaggaggaactgacgcagctacgccatgaactggagcggcagaacaatgaataccaagtgctgctgggcatcaaaacccacctggagaaggaaatcaccacgtaccgacggctcctggagggagagagtgaagggacacgggaagaatcaaagtcgagcatgaaagtgtctgcaactccaaagatcaaggccataacccaggagaccatcaacggaagattagttctttgtcaagtgaatgaaatccaaaagcacgcatgagaccaatgaaagtttccgcctgttgtaaaatctattttcccccaaggaaagtccttgcacagacaccagtgagtgagttctaaaagatacccttggaattatcagactcagaaacttttattttttttttctgtaacagtctcaccagacttctcataatgctcttaatatattgcacttttctaatcaaagtgcgagtttatgagggtaaagctctactttcctactgcagccttcagattctcatcattttgcatctattttgtagccaataaaactccgcactagctgcaaaaaaaaaaaaaaaaaaa

SEQ ID NO: 8 - RNA, 4048 nt, Homo sapiens
  source 1..4048: mol_type = mRNA
ggaagtgggcctggcaccttcccggcctgccgcagggatggggcagctgtgctggctgccgctgctggcaccgctcctgttgctgcgaccgccaggggtccagtccgccggccccatccgggccttcgtggtgccccacagccacatggacgtgggctgggtctacactgtgcaggaaagcatgcgggcgtacgccgccaatgtctacacctcagtggtggaagagctggcccgcggccagcagcgccggttcatcgctgtggagcaggagtttttccggctgtggtgggatggcgtcgcctcggaccagcagaaataccaggtccgccagctcctggaggaaggacgcctggaatttgtcatcggaggccaggtcatgcatgacgaggctgtgacgcaccttgatgaccagatcctgcagctcacagaaggacacgggtttctctatgaaacatttgggatccggccacagttctcctggcacgttgacccgtttggcgcctctgccacgacgcccaccctatttgcgctggcgggcttcaatgcccacctcggctcccggatcgactacgacctgaaggcagccatgcaggaggcccgggggctgcagttcgtgtggcgagggtccccatccctctcagagcggcaggaaatcttcacgcacatcatggaccagtacagctactgcaccccgtcccacatccctttctccaacaggtcaggattttactggaatggcgtggctgtcttccccaagcctccccaagatggggtgtaccccaacatgagtgagcctgtcaccccagccaacatcaacctctatgccgagctcggtgtctcggtgcagtatgccacgctgggcgactacttccgtgccctgcacgctctcaatgtcacctggcgtgtccgcgaccaccacgacttcctgccctattccacagaaccattccaggcctggacgggcttctacacgtcccgcagctcactgaaggggctggcccggcgagccagcgccttgttgtatgccggggagtccatgttcacacgctacctgtggccggccccccgtgggcatctggaccccacctgggccctgcagcagctccagcagcttcgctgggccgtctccgaggtccagcaccatgatgccatcactgggactgagtcccccaaggtgagagacatgtacgcaacgcacctggcctcggggatgctgggcatgcgcaagctgatggcctccatcgtcctagatgagctccagccccaggcacccatggcggccagctccgatgcaggacctgcaggacattttgcctcggtctacaacccgctggcctggacggtcaccaccatcgtcaccctgactgttggtttccctggagtccgcgtcacagatgaggcgggccacccagtgccctcgcagatccagaactcaacagagaccccatctgcgtatgacctgcttattctgaccacaatcccaggcctcagttaccggcactacaacatcagacccactgcaggggcccaagagggcacccaggagccggctgccactgtggcgagcacccttcaatttggccgcaggctgaggagacgcaccagccatgcgggcaggtacttggtgcctgtggcaaacgactgctacattgtgctgctcgaccaggataccaacctgatgcacagcatctgggagagacagagtaaccgaacggtgcgcgtgacccaggaattcctggagtaccacgtcaacggggatgtgaaacagggccccatttccgataactacctgttcacaccgggcaaggccgcggtgcctgcgtgggaagctgtggaaatggagattgtggcgggacagcttgtgactgagatccggcagtacttctacaggaacatgacagcacagaattacacgtatgcaatccgctcccggctcacccatgtgccgcagggccatgacggggagctgctctgccaccggatagagcaggagtaccaagccggccccctggagctgaaccgtgaggctgtcctgaggaccagcaccaacctaaacagccagcaggtcatctactcagacaacaacggctaccagatgcagcggaggccctacgtttcctatgtgaacaacagcatcgcccggaattactaccccatggttcagtcggccttcatggaggatggcaaaagcaggcttgtgttgctgtcggagcgggcacatggcatctccagccaagggaatgggcaggtggaggtcatgctccaccggcggctgtggaacaacttcgactgggacctgggctacaacctcacgctgaacgacacctcagtcgtccacccagtgctctggcttctgctgggatcctggtccctcaccactgccctgcgccagaggagcgcactggcgctgcagcacaggcccgtggtgctgttcggagacctcgctgggactgcgccgaagctcccaggaccccagcagcaagaggccgtgacgctgcccccgaatcttcacctgcagatcctgagcatccctggctggcgctacagctccaaccacacggagcactctcagaatctccggaaaggccatcgaggggaagcccaggctgacctccgccgtgtcctgctgcggctctaccacctgtatgaagtgggcgaggacccagtcctgtctcagccagtgacagtgaatctggaggctgtgctgcaggcgctggggtccgtggtggcagtggaggagcgctcgctcacagggacctgggatttgagcatgctgcaccgctggagctggaggacggggcctggccgccacagaggtgacaccacctctccctcgaggccaccaggaggccccatcatcaccgtccacccaaaggaaatccggacgttctttattcactttcaacagcagtgagccctgggcagatgccccggccccagggcttcccccaggaactccatgtaacagaacagacccaggacagggaaaagcagtgcggagggatgggactggggagtcagctgctcatctgcaggctaatggcaggaaatggtcatatttggggtttttccctaatttttttaaacaaaaattacattacaagatccaggttcttcccccccacactcaatcaagccagccctctcctcttctgtcacgtaaaggatatttggcacactcatgcgtcattcattcacaaaacacaaacccaggactttctgcctaaggcagagcacaagactcacagcagcaccgaagcgcatctgccgtccgggccctgccaggcttgccaggctgccagtggtaactgtggacctactgcgtgccacgtgttttcatagactcatcccatgctggcaacagccctgcaaggggcttggctctgccacagggcaggagaggaagttgtagcgcctagcgagagttccagccccagacgcccacctgtgcctcagggcaccgcctgccgagcagagaaggcacagcagccgtcagagtccatgagaggtgaaaccacacagcagggatgtccaatatcagaactattaatatcaataaaagtataaccttcccaggtctatgcccaagagaattgaaaacatccatccacacaatacctgtgctcccgcgttcatagcagcattactcaaaagtcaaacggtagcaacaacccaaatgtccatccacagatgaattaagacatgaagtgtgttctgtccatacaatggaatattatttggccataaaaaggaaggaaattctgacgcatgccacagcctgagtgaatcctacaaatattacgctaagtgaaagaagccaatcacgagtttatgtgaaatgtccagaataggcaaatctgtgtatcagagacaaagcacattggtggttgccaggtactggaggaagagagaagaggcatgacagctaacagggacgggctttctttggaagatgatgaaattgtggaatgatggttgcacaactttgtgaatatactagaaaccaatgaattaaaaactttggaagatgaaaaaaaaaaaaaaaaaa

SEQ ID NO: 9 - RNA, 2141 nt, Homo sapiens
  source 1..2141: mol_type = mRNA
tgcgtgccgccgttgatccggtgctgcagtgaggaggtggttcttgcccgtgttgtgtgtgtgtgtgagtgagagagcgagtgagtgagtgagtgagtgtgtgtgtgggggggactcggcttgttgttgtcggtgacttccccctccccttcaccccttcccctccccgccgccgctgcagtggccgctccctgggccgtaggaaatgagcgataacgatgacatcgaggtggagagcgacgaagagcaaccgaggtttcaatctgcggctgacaaacgggctcatcataatgcactggaacgaaaacgtagggaccacatcaaagacagctttcacagtttgcgggactcagtcccatcactccaaggagagaaggcatcccgggcccaaatcctagacaaagccacagaatatatccagtatatgcgaaggaaaaaccacacacaccagcaagatattgacgacctcaagcggcagaatgctcttctggagcagcaaggggaaagcgagagctgatcaagttctttgttcctggggaattcacttctcttcctccctcatggaagatgcaagtaaaaggaaattccgtgcactggagaaggcgaggtcaagtgcccaactgcagaccaactacccctcctcagacaacagcctctacaccaacgccaagggcagcaccatctctgccttcgatgggggctcggactccagctcggagtctgagcctgaagagccccaaagcaggaagaagctccggatggaggccagctaagccactcggggcaggccagcaataaaaactgtctgtctccatcgtctcatcctcctttcagttcgttggtagagccctcagaaccatttaagagactctttatttttctctttctcccttttttttttaaatttttatttttacgtagaagctcttggacaacagctctcgttctccttccccatttccactgtatattttttaatgtattcccttcagggattccctgtccccaacaggaatttttaaaccaaaacaccccaacttggcagctttttctgtggaggacagacggccggccggacctctgagcacatagtgtcctgcccaccctaccagctcctccagccctgccgggcacatgcccgggggacgcctgccctgcccaggtggcctcctggcccgccctcacctctgatagactttgtgaatctgaactgctctactttgagaagatgaccggtttggagtaatcagaatgaaccctcctcctttttaagggttttttttttttcctttttctaaaaagctatgtatcgctcctattgaaagaccagatccttagagaagtttgtggtataaaaaggaagtggggacagattcgcagcacagagtcgctggcatgtttcactcctgcttctctcagccagctgtttaagcctgcggcgccagcctcacggagggccgtgtgacactctcgtggtatgtatgggagatggcagcagtgaagcagcagccaccagggagtggccatttggggttgggacagggagggtgttttgggtggcatagaggttttgtattgagggccagtgatgatgttttgatatttatttcctgctacttaaatttgaatctgagtgaattgtacctatttctgatgatgtcggtcttgcaaagcgacagattcataaagtaatgatgaaatctttctttcttcccgtgtgtatttctaagaaatagagccaactgattttgtatgtaaataccaagagcaatttacctggtactaaacccgcaccccagtgcggacccttcccagccctcatcccacttcctttcctactgtcctggaacctgtctccattgtgtgatccagccctggttctggctgtggtcagcagatgccagtgaagggttttgtgtgtttaggcctcatttctttgtctttttcctactccgttcctggcatttgctgatttctagtgtatactctgtagtctcagttcgtgtttgattccattccatggaaataaaaagtatgttgtacatactgccgaagaattgtcttgcaagttaaggcttccccctttactataagactataaataaaaacttattttatccttcttaaaaaaaaaaaaaaaa

SEQ ID NO: 10 - RNA, 4600 nt, Homo sapiens
  source 1..4600: mol_type = mRNA
gaagcgtctcctgtgcgtctgcgcgggaagtggaacctggctctggggagaagccgcgtgagatccgcgcgggtgctagctagtcctttctcgtcgctgctcggctcgcggcccgtggggtcggccccgccaccgttgccgccatgcccatgaagggccgcttccccatccgccgcaccctgcaatatctgagccaggggaacgtggtgttcaaggactccgtgaaggtcatgacagtgattacaacacgcatggggagctgggcgagggcgccaggaagtttgtgtttttcaacatacctcagattcaatacaaaaacccttgggtgcagatcatgatgtttaagaacatgacgccgtcacccttcctgcgattctacttagattctggggagcaggtcctggtggatgtggagaccaagagcaataaggagatcatggagcacatcagaaaaatcttggggaagaatgaggaaaccctcagggaagaggaggaggagaaaaagcagctttctcacccagccaacttcggccctcgaagtactgcctgcgggagtgcatctgtgaagtggaagggcaggtgccctgccccagcctggtgccattacccaaggagatgagggggaagtacaaagccgctctgaaagccgatgcccaggactaaggcccacggtcactgtgggctggggtgatggtgtctgaccagtggggagattggaatgggattactttggcccagggaagcccctggttctgtccctggagactctggaaatccttttgcattaaaaggactttacacacctgtgtaaaaggatgtgggagaggagggtctgaagctgagctgctaaatgaatatccctgctctgctggtcaataaaacgcttcctaatagcagcttggcgtgtatctggtcctagtgaagaggaaggcctgtgtagcagaaaggctttgggcctgagaggttaaggccacagcctgttgacacctgttttggtcctgcgaccctttactggtctccgctggctttgaatcttcctctgggctctactctggagaacataagggctgctgtggttgagtctggctagcactgtctgtggttggcagtgtgtacacccctccgttcagttccttgggggtatttttcagaaatccaaaggcaacccttcgtgcagtgctcacttttttaagtacagttgattacccttgcctgctggggggcctagccatgggccagagatggaggagccccagtggctgacaggccagcctcactcaggcacgtacctgctgaccagtcagccactgccaacccatggcccagccactgtgtgcattagcagggaggtttgtaggccatggaggaaatgaggagacaccacctagtggagacattggggccctgctggggggatggtgtctatagctggctctgctggctccctcaggccctgcttaccaagctctggaggagggagtgctgcattactgagcaccttccttgttctttcctcataggacactgatgttactgtcactttagttatgctaaagtggaggtttcagcctccagaagacagcagagccttctagggtcaccttaagaataggtttagctaggctgggtgtggtggctcatgcctgtaatcccagcactttgggaggctgatgtgggcggatagttgagcccaggagttcgagaccagcctgggcaacatggcaagaccttgtctctacaaaaagtacaaaaattagatgggtgtggtgtttcgcgtctgtagtcccagctatttgggaagctgaggtgggaggatctcgagcctgcgagatcaaggctgcagtgaatcatgatcgtgcgctactgcactccagcctgggccacagagcaagactgtctcaagaaaaaaaattttttaaataagtttaattataaagtgaaggaccagttggaccacggacccctagaaactcagccaaggagacctgactttatctgagacaggaaggcagtggttaaaagaattaaaaaaggacagtggtgcttgacagagcaggactgaagttttcattcctctacctgctggggccttggtcaagtcctaaaagctttccaggtctgtaactggttttgtctgtaaaatggggaaaaggttaccttttctgccacatgaagtagctggctggtgaaatgatgtcatactacgatgtgtttcattaactgttggagattatcacattaaattgagcagatttggatcttttacatttgatgctcaagtttgattctgtaccagatctgggtttggggtgatggcttgtcgggggtgggaggggaaggtgggcagagactgatgtataatggacaggtgttccttgagtgacagacttacaaagggagtgtttaattgaaatctagcatcttgtctctctgctatcagcattttggctgggtggaaaaagctctcctccacagagagccctcccatgtctgctggtttcagttccccacccaactgactgaagtcttgaccttgataccacctgcctgggcgttctacttggctgcaagggctcttcagaaagcacctagttccttttggctttcatctgcagtgtaggtctccatagctagtgacacacaaagccttatgagagtattccttccctctagggcccaaagaggctgctaaccagcaggaatgtcctgaaccaaaaggagtggtgaccattctctagacagaataaatgtctggtccctaatgggacagataactagggtttgagttcaggcttgacacaggagcaatgggcagtgaggtggacagcccagaccctgaaaggacgtgtggagacagcagcagtcctgtggtgcagttactgcctgtgtggccagcttcacaggcagcagataatttgtggaaataaacattattttctttttttgttccctgtaggctacttgatcctggatagtttctctttaaacctttgtctttccctattttgtttagtagtgatgaaagatgggtttaaaatatacagaaatgggagaaaattaattagagaaaatgtgaggattttataattgataacatgttcttaattttacttgcacaagcagtgttcacagccttattctgaagtgaccacttttcttctctttgaggcactacctggcagcaccatctaaacctgtatcatctgctgtggtcccagttactcaggaggctgaggcaggaggatcacttgagcccaggaggttgaggctggagtgagccatgactgtaccactgcattccagcctgggtgacggagtgaggccctatctgcctgccagtgagttctgtcatgcatcagactttggacctcagaattttcacctggaacatgagaggtcagatgacataatgaatatgttctaacttctgaaacttttggagcttgtttttgcccttgatagcacgtatccttagagaataagcttgccaatttaaggcctgtctgtaatggcatctgaatgttccagcagcctaactgtgctctgctccagaaacatgtgtgtggcttccctacctccacaccaaggagctgccttgctgggtggatgtaaagactgtcctgccagggtttcccactaaaggtattaataagtccagtggaccacagagggcccaatccctcttggccgctaaggctaaggtaggagccagagagctgtagagacaggaccaccaggatgcttcatgtccataacacaagagagcgctgcattgtttgggggccaggcattactactatcatgaagaaaaatgaagagcaggatgcatgttagtctttaattcacttttaatagtataaacctcatttaggtagtagtattaagccacaacaataatgccacattgaaacagcatttaataaaatgcataaagctaattcatgcactgcaatactctatatacaaacacaacaatgcaaattcttcttcaagactgaagacattgattagatataaaattcagtttaaaaagaacatgctattttttaaatgccatcacataaacaaagtgatttcacagggagaagaaagctgtataaagctgcagctttcaacaggttttaaacctggcattaaaatgtaatggcaaaaccaatattttctatattgtgaagggcaaaggttacagaaacggtcccaagaaaatctaacacccaaattttcctttgatagatagatgcctttaaaagggctggtaatgcagttacattctaacagagaagtccaaactacaggtaaaaactacggcttgtactgtgaaaaatgtgcagcttttcagttataaaactagttgaacactggtttacaagataatccgtagaacagagagactgtagaaaaatattccagcacttgagttgtgtgtggcagcagcatttgagtccatgaatgctatggtcattaaaataattgattatactttcctaaagaaaagccattttttaatgcaaagtctactacttccaaatgttattccaaacattaaattttaacagtctatcaatcaataaatgttgaacattttttttctaaaaaaaaa

SEQ ID NO: 11 - RNA, 2082 nt, Homo sapiens
  source 1..2082: mol_type = mRNA
aaaagccagccaggagctgtgggaggaaacgccctcagtaaagatgaccgcggtcactgttatctaaacgcaagtgaagccgagtcacaggacccggatgttgtcagttcgacggtaaacgaccctgccagcttccaagagggcggcttcactgtgcgaataggtgagaagccaagaaggaggcgcgctggagttacttccgcccggttctccttcccgcagtctgcagccggagtaagatggcggcgctgagggctttgtgcggcttccggggcgtcgcggcccaggtgctgcggcctggggctggagtccgattgccgattcagcccagcagaggtgttcggcagtggcagccagatgtggaatgggcacagcagtttgggggagctgttatgtacccaagcaaagaaacagcccactggaagcctccaccttggaatgatgtggaccctccaaaggacacaattgtgaagaacattaccctgaactttgggccccaacacccagcagcgcatggtgtcctgcgactagtgatggaattgagtggggagatggtgcggaagtgtgatcctcacatcgggctcctgcaccgaggcactgagaagctcattgaatacaagacctatcttcaggcccttccatactttgaccggctagactatgtgtccatgatgtgtaacgaacaggcctattctctagctgtggagaagttgctaaacatccggcctcctcctcgggcacagtggatccgagtgctgtttggagaaatcacacgtttgttgaaccacatcatggctgtgaccacacatgccctggaccttggggccatgacccctttcttctggctgtttgaagaaagggagaagatgtttgagttctacgagcgagtgtctggagcccgaatgcatgctgcttatatccggccaggaggagtgcaccaggacctaccccttgggcttatggatgacatttatcagttttctaagaacttctctcttcggcttgatgagttggaggagttgctgaccaacaataggatctggcgaaatcggacaattgacattggggttgtaacagcagaagaagcacttaactatggttttagtggagtgatgcttcggggctcaggcatccagtgggacctgcggaagacccagccctatgatgtttacgaccaggttgagtttgatgttcctgttggttctcgaggggactgctatgataggtacctgtgccgggtggaggagatgcgccagtccctgagaattatcgcacagtgtctaaacaagatgcctcctggggagatcaaggttgatgatgccaaagtgtctccacctaagcgagcagagatgaagacttccatggagtcactgattcatcactttaagttgtatactgagggctaccaagttcctccaggagccacatatactgccattgaggctcccaagggagagtttggggtgtacctggtgtctgatggcagcagccgcccttatcgatgcaagatcaaggctcctggttttgcccatctggctggtttggacaagatgtctaagggacacatgttggcagatgtcgttgccatcataggtacgaggcctattgtgtagtagaggtatcctagacaaaggagttcgggacgcccactggggacagaaggagaacacttcctgttcaccataggccatggcatggactcgggtcctcaatcttttgagcacagtaatgggttctggatcttgggtaacaccactttttttgtttgttttgcctcacaacaggaagataagtaacatcacttttttcctccatcctctcacctaggtacccaagatattgtatttggagaagtagatcggtgagcaggggagcagcgtttgatcccccctgcctatcagcttcttctgtggagcctgttcctcactggaaattggcctctgtgtgtgtgtgtgtgtgtgtgtgtgtgtgtatgttcatgtacacttggctgtcaggctttctgtgcatgtactaaaaaaggagaaattataataaattagccgtcttgcggcccctaggcctaaaaaaaaaaaaaaaaaa

SEQ ID NO: 12 - RNA, 4598 nt, Homo sapiens
  source 1..4598: mol_type = mRNA
gtagttcctttcccacaatcggctgggcgaggcggcgccagcgatcagagcagcgctgggtgttcaggggccaagatggcggcgcgccggggacggagagacggagtcgcgccgcccccgagtgggggccccggtccggaccctggcgggggagcccgcggcagtggttggggaagtcgaagccaagcgccgtatgggactttgggcgctgtgagcggcggcgagcaggtgctgctgcatgaggaggcgggtgattctggctttgtcagtctctctcggctgggcccatctctgagggacaaggacctggaaatggaggagctaatgctgcaggatgagacactgctggggaccatgcagagctacatggatgcctcccttatctccctcattgaggattttgggagccttggagagagcaggttatctctggaggaccagaatgaagtgtcgctgctcacggctctgacggagatcttggacaatgcagattctgagaacctttctccatttgacagcattcctgattcggagctgcttgtgtcaccccgggagggctcctctctgcacaagctgcttactctctctcggacacccccagaacgtgacctcatcaccccagttgacccactggggcccagtacaggcagcagtagagggagtggggttgaaatgtctcttccagatccctcttgggacttctccccaccctctttcttagagacctcttcccccaagcttcctagctggagacccccaagatcaagaccacgctggggccaatccccacctccccagcagcgcagtgatggagaagaagaggaggaggtggccagcttcagtggccagattcttgccggggagcttgacaactgtgtgagcagtatcccggacttccccatgcatttggcctgccctgaggaggaagataaagcaacagcagcagagatggcagtgccagcagctggtgatgagagcatctcctccctgagtgagctggtgcgggccatgcacccatactgcctgcccaacctcacccacctggcatcacttgaggatgagcttcaggagcagccagatgatttgacactgcctgagggctgcgtagtgctggagattgtggggcaggcagccacagctggcgatgacctggagatcccagttgtggtgcgacaggtctctcctggaccccggcctgtgctcctggatgactcgctagagactagttctgccttgcagctgcttatgcctacactggagtcagagacagaggctgctgtgcccaaggtaaccctctgctctgagaaagaggggttgtcattgaactcagaggagaagctggactcagcctgcttattgaagcccagggaggtcgtggagccggtggtgcccaaggagcctcagaacccacctgccaatgcagcaccaggttcccagagagctcgaaagggcaggaagaagaagagcaaggagcagccagcagcctgtgtggaaggctatgccaggaggctgaggtcatcttctcgcgggcagtctactgtaggtacagaagtgacctctcaggtagacaacttgcagaaacagcctcaggaagaacttcaaaaagagtctgggcctctccagggtaaggggaagccccgggcttgggctcgggcctgggcagctgccttggagaattctagccctaagaacttggagagaagtgctggacaaagtagtcctgctaaagaaggccctctagacctctacccaaagctggctgacactatccaaaccaatcctataccaacccatctctcattggtcgactctgcccaagccagccccatgccagttgactctgttgaagctgatcccactgcagttggccctgttctagctggccctgtacctgttgaccctgggttggttgaccttgcttcaaccagctcagaactggttgagcctctcccggctgagccagtgctgatcaacccagtcctggctgactcagcagcagttgaccctgcagtggttcccatctcagataacttgccaccagttgatgctgtcccgtctggcccagcaccagttgatctagcactggttgaccctgttcctaatgacctgactccagttgacccagtgctagttaagtccagaccaactgatcccagacgtggtgcagtgtcatcagccctggggggttcagcaccccagctcctcgtggagtcagagtccttggacccaccaaagaccatcatccctgaagtcaaagaggttgtggattctctgaaaattgaaagtggtaccagtgctacaacccatgaagccagacctcggcctctcagcttatctgagtaccggcgacgaaggcagcaacgccaagcagaaacagaagagagaagtccacagcccccaactgggaagtggcctagccttccagagactcccacagggctggcagacatcccttgtcttgtcatcccaccagccccagccaagaagacagctctgcagagaagccctgaaacaccccttgagatttgccttgtgcctgtaggtcccagccctgcttctcctagtcctgagccacctgtaagcaaacctgtggcctcatctcccactgagcaggtgccatcccaggagatgccactgttggcgagaccttcccctcctgtgcagtctgtgtcccctgctgtgcccacacctccctcgatgtctgctgccctgcctttccctgcaggtgggcttggcatgccccccagtctgcccccacctcccttgcagcctcctagtcttccattgtctatggggccagtactacctgatccgtttactcactatgcccccttgccatcctggccttgttatcctcatgtgtccccttctggctatccttgcctgccccccccaccaacggtgcccctagtgtctggtactcctggtgcctatgccgtgcctcccacttgcagtgtgccttgggcaccccctcctgccccagtctcaccttacagttccacatgtacctatgggcccttgggatggggcccagggcctcaacatgctccattctggtctactgttcccccacctcctttgcctccagcctccattgggagagctgttccccaacctaaaatggagtctaggggcactccagctggccctcctgaaaatgtacttcccttgtcgatggctcctcccctcagtcttgggctacctggccatggagctcctcagacagagcctaccaaggtggaggtcaagccagtgcctgcatctccccatccgaaacacaaggtgtctgccctggtgcaaagtccccagatgaaggctctagcatgtgtgtctgctgaaggtgtgactgttgaggagcctgcatcagagaggctaaagcctgagacccaagagaccaggcccagggagaagccccccttgcctgctaccaaggctgttcccacaccaaggcagagcactgtccccaagctgcctgctgtccacccagcccgtctaaggaagctgtccttcctgcctaccccacgtactcagggttctgaagatgtggtacaggctttcatcagtgagattggaattgaggcatcggacctgtccagtctgctggagcagtttgagaaatcagaagccaaaaaggagtgtcctcctccggctcctgctgacagcttggctgtaggaaactcagggtccagctgtagttcctctggacgttctcgaagatgctcttcctcttcttcgtcatcatcttcctcttcgtcttcctcatcctcatcatccagttctcgaagccgctcacgatccccatccccccgccggagaagtgacaggaggcggcggtacagctcttatcgttcacatgaccattaccaaaggcaaagagtgctacaaaaggagcgtgcaatagaagaaagaagggtggtcttcattggaaagatacctggccgcatgactcgatcagagctgaaacagaggttctccgtttttggagagattgaggagtgcaccatccacttccgtgtccaaggggacaactacggcttcgtcacttatcgctatgctgaggaggcatttgcagccattgagagtggccacaagctgcggcaggcagatgagcagccctttgatctctgctttgggggccgaaggcagttctgcaagaggagctattctgatcttgactccaaccgggaagactttgacccagcacctgtaaagagcaaatttgattctcttgactttgacacattgttgaaacaggcccagaagaacctcaggaggtaaccttgggcccttccctgctatcctttttctcctttggaggtgcccaacctcctccacccccttcccctactctaggggagagagctgctagtgagatgactgttttataaagaaatggaaaaaagtgaaataaaaaatatgttgaatcagattttttaaaaggggtatttgtttttttataacaggtattgaaacaagttaacttgcattcctatgtaagataggaggggctgaggggatccccagtgtttggaacataagtcactatgcagactaataaacatcaactagagagaactcccaaaaaaaaaaaaaaaaaa

SEQ ID NO: 13 - RNA, 1818 nt, Homo sapiens
  source 1..1818: mol_type = mRNA
ggtggtcagcgcgcgcattgcctgccccggaagtggtcggcgcgcggcgcggcgcgcctgggcgctaagatggcggcggcgtgagttgcatgttgtgtgaggatcccggggccgccgcgtcgctcgggccccgccatggccgtcaccatcacgctcaaaacgctgcagcagcagaccttcaagatccgcatggagcctgacgagacggtgaaggtgctaaaggagaagatagaagctgagaagggtcgtgatgccttccccgtggctggacagaaactcatctatgccggcaagatcttgagtgacgatgtccctatcagggactatcgcatcgatgagaagaactttgtggtcgtcatggtgaccaagaccaaagccggccagggtacctcagcacccccagaggcctcacccacagctgccccagagtcctctacatccttcccgcctgcccccacctcaggcatgtcccatcccccacctgccgccagagaggacaagagcccatcagaggaatccgcccccacgacgtccccagagtctgtgtcaggctctgttccctcttcaggtagcagcgggcgagaggaagacgcggcctccacgctagtgacgggctctgagtatgagacgatgctgacggagatcatgtccatgggctatgagcgagagcgggtcgtggccgccctgagagccagctacaacaacccccaccgagccgtggagtatctgctcacgggaattcctgggagccccgagccggaacacggttctgtccaggagagccaggtatcggagcagccggccacggaagcaggagagaaccccctggagttcctgcgggaccagccccagttccagaacatgcggcaggtgattcagcagaaccctgcgctgctgcccgccctgctccagcagctgggccaggagaaccctcagcttttacagcaaatcagccggcaccaggagcagttcatccagatgctgaacgagccccctggggagctggcggacatctcagatgtggagggggaggtgggcgccataggagaggaggccccgcagatgaactacatccaggtgacgccgcaggagaaagaagctatagagaggttgaaggccctgggcttcccagagagcctggtcatccaggcctatttcgcgtgtgaaaaaaatgagaacttggctgccaacttcctcctgagtcagaactttgatgacgagtgatgccaggaagccaggccaccgaagcccccaccctacccttattccatgaaagttttataaaagaaaaaatatatatatattcatgtttatttaagaaatggaaaaaaaaatcaaaaatcttaaaaaaacaagcaaacagtccagcttcctgtcctcctaaagtggcccctgttcccatctcccgggccagacagctgtccccccgtcctcctccccagcccagcctgctcagagaagctggcaggactgggaggcgacagatgggcccctcttggcctctgtcccagctctctgcagccagacggaaaggcggctgcttgcctctccatcctccgaaaaacccctgaggacccccccccatcctcttctaggatgaggggaagctggagccccaactttgatcctccattggagtggcccaaatctttccatctagggcaagtcctgaaaggcccaaggccccctccccagtctggccttggcctccagcctggagaagggctaacatcagctcattgtcaaggccacccccaccccagaacagaaccgtgtctctgataaaggttttgaagtgaataaagttttaaaaactag

SEQ ID NO: 14 - RNA, 3310 nt, Homo sapiens
  source 1..3310: mol_type = mRNA
cccggcggggggaggttggggacgggcctggcagttgtgaactcgaacctgccgctgtcgccgcggcggggcggggagcgagagtgggccgcggaggccggccttcgggctccatggacgggcgccgcgtccctgcacagcccgccgcagaggtaaggctggcctctctgcagtcagaggtctgagctctgccatggggataggggtgtctttattactgcagttttctctaacacctgggggctaccggagtgtgggccgaagcaggcgctgcagccgcggaagtatccccaggaacatccccaagaggagctggaaaaagcctcatccccagctctgcagtctccaggggagctcagtgtctgtttgtccagcttctcagagttgctgtgcagctcggatgtggcataggaaacagcagacacagggagagggcagcataaggcactgtagggagcagtggccacattttctgcagaggaagaaccgatgctggaacgtcgttgcaggggccccctggccatgggcctggcccagccccgactcctttctgggccctcccaggagtcaccccagaccctggggaaggagtcccgcgggctgaggcaacaaggcacgtcagtggcccagtctggtgcccaagccccaggcagggcccatcgctgtgcccactgtcgaaggcacttccctggctgggtggctctgtggcttcacacccgccggtgccaggcccggctgcccttgccctgccctgagtgtggccgtcgctttcgccatgcccccttcttagcactgcaccgccaggtccatgctgctgccaccccagacctgggctttgcctgccacctctgtgggcagagcttccgaggctgggtggccctggttctgcatctgcgggcccattcagctgcaaagcggcccatcgcttgtcccaaatgcgagagacgcttctggcgacgaaagcagcttcgagctcatctgcggcggtgccaccctcccgccccggaggcccggcccttcatatgcggcaactgtggccggagctttgcccagtgggaccagctagttgcccacaagcgggtgcacgtagctgaggccctggaggaggccgcagccaaggctctggggccccggcccaggggccgccccgcggtgaccgccccccggcccggtggagatgccgtcgaccgccccttccagtgtgcctgttgtggcaagcgcttccggcacaagcccaacttgatcgctcaccgccgcgtgcacacgggcgagcggccccaccagtgccccgagtgcgggaagcgctttaccaataagccctatctgacttcgcaccggcgcatccacaccggcgagaagccctacccgtgcaaagagtgcggccgccgcttccggcacaaacccaacctgctgtctcacagcaagattcacaagcgatccgaggggtcggcccaggccgcccccggcccggggagcccccagctgccagccggcccccaggagtccgcggccgagcccaccccggcggtacctctgaaaccggcccaggagccgccgccaggggccccgccagagcacccgcaggacccgatcgaagcccccccctccctctacagctgcgacgactgcggcaggagcttccggctggagcgcttcctgcgggcccaccagcggcagcacaccggggagcggcccttcacctgcgccgagtgcgggaagaacttcggcaagaagacgcacctggtggcgcactcgcgcgtgcactccggcgagcggcccttcgcctgcgaggagtgcggccgccgcttctcccagggcagccatctggcggcgcatcggcgcgaccacgcccccgatcggcccttcgtgtgtcccgactgcggcaaggccttccgccacaaaccctacctggcggcgcaccggcgcatccacaccggcgagaagccctacgtctgccccgactgcggcaaagccttcagccagaagtccaacctggtgtcgcaccggcgcatccacacgggcgagcggccctacgcctgtcccgactgcgaccgcagcttcagccagaagtccaacctcatcacccaccgcaagagccacatccgggacggcgccttctgctgtgccatctgtggccagaccttcgacgacgaggagagactcctggcccaccagaagaagcacgatgtctgagacggtgggcggggccgtgttggctgagagagggctggggtccttcgtggtgggagtcgcagtgggctgggggtgcctgcctagtgctggagtaggggacaatgggaatcctagaggggatggaagacgcggggagtgagctgggtgggccctgctagcgagagaggtcaaccccggtggccagggaacccacttccaagcgcagggacgccggcctccagctggtgtgtgctaaggctccgtcctgactgccctgtgccctggaaaagcagcaatagcatccgccccttagagccctctggctagaggagccaccagtggaaaggaagaccctccatcctctggtattaacgccttaatgcccctgtcttttactgtaagttacttaagatcatttttggaagcaggcgtggtagagtcctgtaaatgaatgctctgggctagatacagcttggagaacctgctggccttgttagacagcacttgggcctttgccagcagcaagaggtgaagcgaagccactcttacctctcccttcccctcccacctgccccctgcgtaggcacccagacttggagagacccgtctgctgttaatacttccatcctcttccttcccaaagagcagatcccaaggcatttactccttggtctgtctcgctttatctgtcgcccctcccagcgctgagagcctcccctggctgtcagcagcactgtgtccaggctcttgtctgaacaccgcagcccctccttcgctccttccagagctcagcatgtcacggcaaggactgccgcattggtgatggagggccagctgaggggaagttgctggtgagtttccttttctccatttctagcatatggacacctggcctctgcttgagcacttaggtgacaggaacttccgcacctcctgaggccctggatgattctaattgttagaaattctaattgttagaaatccttccttataatgaatgaattctgctttcctataatttctacctattgggccttgttctgttctctggaactaaacagaacaaccatttacccctccttttcaaactagagaataaagatttggttttagaactggtaaaaaaaaaaaaaaaaaa

SEQ ID NO: 15 - RNA, 1246 nt, Homo sapiens
  source 1..1246: mol_type = mRNA
actttacgcaggcgcgttaggtcctagtcgctatgcgtgtgcttgtgggtggcgggacagcttcattgggacagccctaacccagctgctgaatgccagaggccacgaagtgacgttggtctcccgaaagcccgggcccggccggatcacgtgggatgagctcgctgcatcggggctgccgagctgcgatgccgccgtcaacctggccggagagaacatcctcaaccctctccgaagatggaatgaaaccttccaaaaagaggtaatcggcagccgcctagagaccacccaattgctggctaaagccatcaccaaagccccacaaccccccaaggcctgggtcttagtcacaggtgtagcttactaccagcccagtctgactgcggagtatgatgaagacagcccaggaggggactttgactttttctccaacctcgtaaccaaatgggaagctgcagccaggcttcctggagattctacacgccaggtggtggtgcgctcaggggttgtgctgggccgtgggggtggtgccatgggccacatgctgctgccctttcgcctgggcctggggggccccatcggctcaggccaccaattcttcccctggatacacatcggggacctggcaggaatcctgacccatgcccttgaagcaaaccacgtgcacggggtcctgaatggagtggctccatcctccgccactaatgctgagtttgcccagaccttgggtgctgccctgggccgccgagccttcatccctctccccagcgctgtggtgcaagctgtctttgggcgacagcgtgccatcatgctgctggagggccagaaggtgatcccacagcgaacactggccactggctaccagtattccttcccagagctaggggctgccttaaaggaaattgtagcctaagtaggtcgtggcaagggcctgaggcctgttcctcacaggcttccaggttaggcactgtgaataggctcagctcctctagagagctgaagccatctggttcttagattcctctcccagtcctctttcccattgttctgttgctccaccttattgtctcaaggccgtaatctcatcaggttgggacattaatcttttcaactccttgtaagatttcccagtttggtttctctacatgtcctgcagctgccccacttctcctttacgctgtgtagagaatgctctgcagtttaggcaataaaaataaattgtctcactaaaaaaaaaaaaaaaaaa

SEQ ID NO: 16 - RNA, 1803 nt, Homo sapiens
  source 1..1803: mol_type = mRNA
aagcgcggggccggccggggcgcccgccgctcgccgccgcctccgcgcgcccgggggccccggcgccccccgagctagggcggccagctcgcggctcgccgtttgacagatgctcatcgccatggagttgccgcagcagcacctttgggggctcgggcgagcgacgggagccgggatctgagcgagcgccggggccagcggagccggagccgccgggacatggttgcagatctgatctcttctgaacacctcatcgtgtctccatccctgggaatctgaccctagcaactggaccactttgttcttggaattttgggtgtcctcttttctcacctttcccttttcccttttccccttccccctcctgagaactccggaagactgtagagattgtcatggagtccagggaaaccttaagcagctcccggcaaagagggggcgagtcagacttcctgccggtctcctcagccaagcccccagctgctcctggctgtgcaggagaacctttgctctccactccaggacctgggaaggggatcccggtgggcggagagcgcatggagccagaggaggaggatgaactaggctcagggcgggatgtggattccaactccaacgcggacagtgagaaatgggtggcaggagatggtttggaagagcaggaattttctatcaaggaggcaaacttcacagagggaagtctgaagctaaagattcagaccacaaagcgggctaagaaacccccaaagaatttggagaactatatatgtccacctgagatcaagatcaccatcaagcagtctggggaccagaaggtgtcccgtgctggaaaaaatagcaaagccacgaaggaggaagaaagaagccactccaaaaagaagctcctcacagccagtgaccttgcagccagtgacctcaaaggatttcagccacagattaaagactccagtaaggaggaagtctggaagagaagaggaggccaaggcatcccattcaaaaagcaattcctgtcccaggaacgtgccatgtgcttctcatgcccccggaacccattccccgcaaaacccggttctctcactcttccttttcacagtgaacctgcagtctgggcacaagaagtataacttcgcatggattctgcaaagcccacacctgtggtcattccctgttctttccattcaacaatggagacttgcccaagattgtaaactagtgagtgacagcatttgggcttatgatcttctctgcctgctagctagaattctctctgggtctaaaagataatccaaaaagatccagcttcacaatgctgccctgaagagataatgcattaggcggccctgatgcagcattcactgtcttccagggcaggcttgatcccatgagtttgcttgctcagacgatcacttaggaaacacatgcctttactctctaggccttttttctagcttgccttgtatcagctatgccatggatctttgctctcttaccccatgctatacagagtatgggctccaagccacagctggcctgtcaagtgtgtgtcgctggtccaccatgggatacatttagaaacttttatagcaatttgacatttttgtgatatccaagcatgtgattgttttcctacggatttgtcttatagtattttaccaaagtttccacacaaaaagtatggattaaggacaaagtatctggtccttcatcaaagatcgtttgataagctctgttctagttaaccaacactgagctttcctagttttaataaaagagtaggatttggaaaaaaaaaaaaaaaaaaaa

SEQ ID NO: 17 - RNA, 3885 nt, Homo sapiens
  source 1..3885: mol_type = mRNA
agagccggacggggtaaactgagcggcggcggcggggcgctggggcggagactgcgacccggagccgcccggactgacggagcccactgcggtgcgggcgttggcgcgggcacggaggacccgggcaggcatcgcaagcgaccccgagcggagccccggagccatggccctgagcgagctggcgctggtccgctggctgcaggagagccgccgctcgcggaagctcatcctgttcatcgtgttcctggcgctgctgctggacaacatgctgctcactgtcgtggtccccatcatcccaagttatctgtacagcattaagcatgagaagaatgctacagaaatccagacggccaggccagtgcacactgcctccatctcagacagcttccagagcatcttctcctattatgataactcgactatggtcaccgggaatgctaccagagacctgacacttcatcagaccgccacacagcacatggtgaccaacgcgtccgctgttccttccgactgtcccagtgaagacaaagacctcctgaatgaaaacgtgcaagttggtctgttgtttgcctcgaaagccaccgtccagctcatcaccaaccctttcataggactactgaccaacagaattggctatccaattcccatatttgcgggattctgcatcatgtttgtctcaacaattatgtttgccttctccagcagctatgccttcctgctgattgccaggtcgctgcagggcatcggctcgtcctgctcctctgtggctgggatgggcatgcttgccagtgtctacacagatgatgaagagagaggcaacgtcatgggaatcgccttgggaggcctggccatgggggtcttagtgggcccccccttcgggagtgtgctctatgagtttgtggggaagacggctccgttcctggtgctggccgccctggtactcttggatggagctattcagctctttgtgctccagccgtcccgggtgcagccagagagtcagaaggggacacccctaaccacgctgctgaaggacccgtacatcctcattgctgcaggctccatctgctttgcaaacatgggcatcgccatgctggagccagccctgcccatctggatgatggagaccatgtgttcccgaaagtggcagctgggcgttgccttcttgccagctagtatctcttatctcattggaaccaatatttttgggatacttgcacacaaaatggggaggtggctttgtgctcttctgggaatgataattgttggagtcagcattttatgtattccatttgcaaaaaacatttatggactcatagctccgaactttggagttggttttgcaattggaatggtggattcgtcaatgatgcctatcatgggctacctcgtagacctgcggcacgtgtccgtctatgggagtgtgtacgccattgcggatgtggcattttgtatggggtatgctataggtccttctgctggtggtgctattgcaaaggcaattggatttccatggctcatgacaattattgggataattgatattctttttgcccctctctgcttttttcttcgaagtccacctgccaaagaagaaaaaatggctattctcatggatcacaactgccctattaaaacaaaaatgtacactcagaataatatccagtcatatccgataggtgaagatgaagaatctgaaagtgactgagatgagatcctcaaaaatcatcaaagtgtttaattgtataaaacagtgtttccagtgacacaactcatccagaactgtcttagtcataccatccatccctggtgaaagagtaaaaccaaaggttattatttcctttccatggttatggtcgattgccaacagccttataaagaaaaagaagcttttctaggggtttgtataaatagtgttgaaactttattttatgtatttaattttattaaatatcatacaatatattttgatgaaataggtattgtgtaaatctataaatatttgaatccaaaccaaatataattttttaacttacattaacaaacatttgggcaaaaatcatattggtaatgagtgtttaaaattaaagcacacattatctctgagactcttccaacaaagagaaactagaatgaagtctgaaaaacagaatcaagtaagacagcatgttatatagtgacactgaatgttatttaacttgtagttactatcaatatatttatgcgttaaacagctagttctctcaagtgtagaggacaagaacttgtgtcagttatcttttgaatccataaatcttagctggcattagttttctatgtaatcacctacctagagagagttgtaaattatatgttaacatgttatctggttggcagcaaacactaaagccaataaaggaaaaacagtaaatgttccgaaagcagagaaaagcaaccaaacatattgttatgaactaaaagctttccctttaagatgcatacttgtcttactggatgaagaaaattgagggtacatgtaccttatactgtcaaggttgtttaaacatgataaggttaatcgccatctacttcaagttttagaaaaggaaacaagaagctgaaaacagctgctctgactttaatatctgactatatctttgatctgtttgcaggtcatccaagtgttttctaggaatatatttattttaggttgtctgaaactactattttttagactcctgaaagttgttcacatcaatgtgaagacaaattttaaatgaaaatgaagaatgaaattatgtcttgaatcatatattaagaagtaaaaataatagtgatcaggcagaaaagaaaaatggaacatctaaaaatgtatgtgctaactatatcatccagtgtgcagtgttgtgtatttttctaagcatgacaacattgatgtgccttttcagtgtaacagcaaatactgttagtgaacattgtcaatttatgtcattttgttaagagatatgactggagtgtgcagtgtggaatgtctctaatactacttgtgaatcctgcagttctataatcataaacaaaaattacttagtttcgttaagctaagattgtgtttgtgttaacttcgacatcaaggagcaaagaactttagaacagactcctcaatcttgtgactttcttattctctaggaaagtaacacttcgtttcatgaagcttttctgtggggcttcgattatttcaagtctggtttctaagtgcagtgtgtttgaagcaaacgaacttccaactcacttatttggcattgggcaacttggccaagtctgccactttggaagatggctctggaggaaactctcatatggctaaaaaggcaggctagtttcttacttctacaggggtagagccttaaaaaagaacgtgctacaaattggttctctttgagggtttctggttctccctgcccccaataccatatactttattgcaattttatttttgcctttacggctctgtgtctttctgcaagaaggcctggcaaaggtatgcctgctgttggtccctcgggataagataaaatataaataaaaccttcagaactgttttggagcaaaagatagcttgtacttggggaaaaaaattctaagttcttttatatgactaatattcttggttagcaagactggaaagaggtgtttttttaaaatgtacataccagaacaaagaacatacagctctctgaacatttattttttgaacagaggtggtttttatgtttggacctggtaatacagatacaaaaactttaatgaggtagcaatgaatattcaactgtttgactgctaagtgtatctgtccatattttagcaagtttacttaataaatcttctgaaccatgaaaaaaaaaaaaa

SEQ ID NO: 18 - RNA, 5486 nt, Homo sapiens
  source 1..5486: mol_type = mRNA
gccctcttctgaagaggcgtttctggaccactgagccccgcctcccactgtgagcggaaccctaccgtttttaaaaaaatctttttcaaaacttgccaggttgtctttccaaatatttttaataatagtgctgctgctgtagaccacagagaaaagaatccctcgctcttccttttcacttagtagaaacttctaccgcgtaggtcccgccaggagttcgcgcatgcgcaggagcgacaataagatggcggtgataatcgccgcactttttttcaaattagtggatcccagaaatcattgcgcgcatttgtaacgaatttccgttcgagtttgtattttaggcgccattttcgagtgaaggacccggagccgaaacaccggtaggagcggggaggtgggtactacacaaccgtctccagccttggtctgagtggactgtcctgcagcgaccatgccccgtaaaggcacccagccctccactgcccggcgcagagaggaagggccgccgccgccgtcccctgacggcgccagcagcgacgcggagcctgagccgccgtccggccgcacggagagcccagccaccgccgcagagactgcaagtgaggaacttgataatagaagtttagaagagattttgaacagcattcctcctcccccgcctccagcaatgaccaatgaagctggagctcctcggcttatgataactcatattgtaaaccagaacttcaaatcctatgctggggagaaaattctgggacctttccataagcgcttttcctgtattatcgggccaaatggcagtggcaaatccaatgttattgattctatgctttttgtgtttggctatcgagcacaaaaaataagatctaaaaaactctcagtattaatacataattctgatgaacacaaggacattcagagttgtacagtagaagttcattttcaaaagataattgataaggaaggggatgattatgaagtcattcctaacagtaatttctatgtatccagaacggcctgcagagataatacttctgtctatcacataagtggaaagaaaaagacatttaaggatgttggaaatcttcttcgaagccatggaattgacttggaccataatagatttttaattttacagggtgaagttgaacaaattgctatgatgaaaccaaaaggccagactgaacacgatgagggtatgcttgaatatttagaagatataattggttgtggacggctaaatgaacctattaaagtcttgtgtcggagagttgaaatattaaatgaacacagaggagagaagttaaacagggtaaagatggtggaaaaggaaaaggatgccttagaaggagagaaaaacatagctatcgaatttcttaccttggaaaatgaaatatttagaaaaaagaatcatgtttgtcaatattatatttatgagttgcagaaacgaattgctgaaatggaaactcaaaaggaaaaaattcatgaagataccaaagaaattaatgagaagagcaatatactatcaaatgaaatgaaagctaagaataaagatgtaaaagatacagaaaagaaactgaataaaattacaaaatttattgaggagaataaagaaaaatttacacagctagatttggaagatgttcaagttagagaaaagttaaaacatgccacgagtaaagccaaaaaactggagaaacaacttcaaaaagataaagaaaaggttgaagaatttaaaagtatacctgccaagagtaacaatatcattaatgaaacaacaaccagaaacaatgccctcgagaaggaaaaagagaaagaagaaaaaaaattaaaggaagttatggatagccttaaacaggaaacacaagggcttcagaaagaaaaagaaagtcgagagaaagaacttatgggtttcagcaaatcggtaaatgaagcacgttcaaagatggatgtagcccagtcagaacttgatatctatctcagtcgtcataatactgcagtgtctcaattaactaaggctaaggaagctctaattgcagcttctgagactctcaaagaaaggaaagctgcaatcagagatatagaaggaaaactccctcaaactgaacaagaattaaaggagaaagaaaaagaacttcaaaaacttacacaagaagaaacaaactttaaaagtttggttcatgatctctttcaaaaagttgaagaagcaaagagctcattagcaatgaatcgaagtagggggaaagtccttgatgcaataattcaagaaaaaaaatctggcaggattccaggaatatatggaagattgggggacttaggagccattgatgaaaaatacgacgtggctatatcatcctgttgtcatgcactggactacattgttgttgattctattgatatagcccaagaatgtgtaaacttccttaaaagacaaaatattggagttgcaacctttataggtttagataagatggctgtatgggcgaaaaagatgaccgaaattcaaactcctgaaaatactcctcgtttatttgatttagtaaaagtaaaagatgagaaaattcgccaagctttttattttgctttacgagataccttagtagctgacaacttggatcaagccacaagagtagcatatcaaaaagatagaagatggagagtggtaactttacagggacaaatcatagaacagtcaggtacaatgactggtggtggaagcaaagtaatgaaaggaagaatgggttcctcacttgttattgaaatctctgaagaagaggtaaacaaaatggaatcacagttgcaaaacgactctaaaaaagcaatgcaaatccaagaacagaaagtacaacttgaagaaagagtagttaagttacggcatagtgaacgagaaatgaggaacacactagaaaaatttactgcaagcatccagcgtttaatagagcaagaagaatatttgaatgtccaagttaaggaacttgaagctaatgtacttgctacagcccctgacaaaaaaaagcagaaattgctagaagaaaacgttagtgctttcaaaacagaatatgatgctgtggctgagaaagctggtaaagtagaagctgaggttaaacgcttacacaataccatcgtagaaatcaataatcataaactcaaggcccaacaagacaaacttgataaaataaataagcaattagatgaatgtgcttctgctattactaaagcccaagtagcaatcaagactgctgacagaaaccttcaaaaggcacaagactctgtcttgcgtacagagaaagaaataaaagatactgagaaagaggtggatgacctaacagcagagctgaaaagtcttgaggacaaagcagcagaggtcgtaaagaatacaaatgctgcagaggaatccttaccagagatccagaaagaacatcgcaatctgcttcaagaattaaaagttattcaagaaaatgaacatgctcttcaaaaagatgcacttagtattaagttgaaacttgaacaaatagatggtcacattgctgaacataattctaaaataaaatattggcacaaagagatttcaaaaatatcactgcatcctatagaagataatcctattgaagagatttcggttctaagcccagaggatcttgaagcgatcaagaatccagattctataacaaatcaaattgcacttttggaagcccggtgtcatgaaatgaaaccaaacctcggtgccatcgcagagtataaaaagaaggaagaattgtatttgcaacgggtagcagaattggacaaaattacttatgaaagagacagttttagacaggcatatgaagatcttcggaaacaaaggcttaatgaatttatggcaggtttttatataataacaaataaattaaaggaaaattaccaaatgcttactttgggaggggacgccgaactcgagcttgtagacagcttggatcctttctctgaaggaatcatgttcagtgttcgaccacctaagaaaagttggaaaaagatcttcaacctttcgggaggagagaaaacacttagttcattggctttagtatttgctcttcaccactacaagcccactcccctttacttcatggatgagattgatgcagcccttgattttaaaaatgtgtccattgttgcattttatatatatgaacaaacaaaaaatgcacagttcataataatttctcttcgaaataatatgtttgagatttcggatagacttattggaatttacaagacatacaacataacaaaaagtgttgctgtaaatccaaaagaaattgcatctaagggactttgttgaactttatgctgaagattcttcaagttgattcagtgtattactgatttttttctatttgtaaaggattatgagttgtataaaatacatactccctaaactagatcatgaaactggtttctgttttatgcagttgtcatttgtaaagtctaataaaatattctctataattgcttctagattacaaaaatatgacaatcttgtaagtagcagactatggagaaaaatgagttacctggagggtcaggtaacttgccaaactaaaaagtatgttagttgaggcaaagtcctaagcaaggttgtgctatcaaggctcagcataccttcgtgggcctttgatttaccaacactggaaatgcctgccaactaatcttggatagattctttaaggcattccacttagcttgccagttgagacaatcaccacagttattacccaaatactatgaacatatttttgtaaaccagtcattctgaattatagtgatgagaatttaaatatatgcttttctagaatttgatgtttgaccatttatgacttaattaccagagagccagtaaattaggacagtgtttcaacaagcctaggctatctcgtaagttgaaaaatatcccactatagttgcttcatgagtatgaagtaagatggcctctgatttacactggttcaatttacaaattttcaactttatgataggtttatccgggtactaaatgcatttcaacttgatagtttcaacttatgataggtttaccaggatgtagtcccactgttgaggagcatctatttaggggttaattactttagtaataagtggaaagtaagataccttgagtaatgtttgcctataaaattgtcagcgtatttttacactattggctcaagaatgttataatgctaagggacataagttggcaaccacttggtttttggaaggactttcggtattgtattagaagtctgccctagctgttaaatttctgggtatttatcctaaggaattaattaaagagttaattgttcctttcttcagtgggccattgttttagatatttaaaaaatccaacagtttctatcataatgtaactgtaaaaatgtaaacacattattagcatggacttttaaataaagatttaaagaaagcaagatcggaaaaaaaaaaaaaaaaaa

SEQ ID NO: 19 - RNA, 3601 nt, Homo sapiens
  source 1..3601: mol_type = mRNA
gggagaaggaggaggccgggggaaggaggagacaggaggaggagggaccacggggtggaggggagatagacccagcccagagctctgagtggtttcctgttgcctgtctctaaacccctccacattcccgcggtccttcagactgcccggagagcgcgctctgcctgccgcctgcctgcctgccactgagggttcccagcaccatgagggcctggatcttctttctcctttgcctggccgggagggccttggcagcccctcaagaagccctgcctgatgagacagaggtggtggaagaaactgtggcagaggtgactgaggtatctgtgggagctaatcctgtccaggtggaagtaggagaatttgatgatggtgcagaggaaaccgaagaggaggtggtggcggaaaatccctgccagaaccaccactgcaaacacggcaaggtgtgcgagctggatgagaacaacacccccatgtgcgtgtgccaggaccccaccagctgcccagcccccattggcgagtttgagaaggtgtgcagcaatgacaacaagaccttcgactcttcctgccacttctttgccacaaagtgcaccctggagggcaccaagaagggccacaagctccacctggactacatcgggccttgcaaatacatccccccttgcctggactctgagctgaccgaattccccctgcgcatgcgggactggctcaagaacgtcctggtcaccctgtatgagagggatgaggacaacaaccttctgactgagaagcagaagctgcgggtgaagaagatccatgagaatgagaagcgcctggaggcaggagaccaccccgtggagctgctggcccgggacttcgagaagaactataacatgtacatcttccctgtacactggcagttcggccagctggaccagcaccccattgacgggtacctctcccacaccgagctggctccactgcgtgctcccctcatccccatggagcattgcaccacccgctttttcgagacctgtgacctggacaatgacaagtacatcgccctggatgagtgggccggctgcttcggcatcaagcagaaggatatcgacaaggatcttgtgatctaaatccactccttccacagtaccggattctctctttaaccctccccttcgtgtttcccccaatgtttaaaatgtttggatggtttgttgttctgcctggagacaaggtgctaacatagatttaagtgaatacattaacggtgctaaaaatgaaaattctaacccaagacatgacattcttagctgtaacttaactattaaggccttttccacacgcattaatagtcccatttttctcttgccatttgtagctttgcccattgtcttattggcacatgggtggacacggatctgctgggctctgccttaaacacacattgcagcttcaacttttctctttagtgttctgtttgaaactaatacttaccgagtcagactttgtgttcatttcatttcagggtcttggctgcctgtgggcttccccaggtggcctggaggtgggcaaagggaagtaacagacacacgatgttgtcaaggatggttttgggactagaggctcagtggtgggagagatccctgcagaacccaccaaccagaacgtggtttgcctgaggctgtaactgagagaaagattctggggctgtgttatgaaaatatagacattctcacataagcccagttcatcaccatttcctcctttacctttcagtgcagtttcttttcacattaggctgttggttcaaacttttgggagcacggactgtcagttctctgggaagtggtcagcgcatcctgcagggcttctcctcctctgtcttttggagaaccagggctcttctcaggggctctagggactgccaggctgtttcagccaggaaggccaaaatcaagagtgagatgtagaaagttgtaaaatagaaaaagtggagttggtgaatcggttgttctttcctcacatttggatgattgtcataaggtttttagcatgttcctccttttcttcaccctccccttttttcttctattaatcaagagaaacttcaaagttaatgggatggtcggatctcacaggctgagaactcgttcacctccaagcatttcatgaaaaagctgcttcttattaatcatacaaactctcaccatgatgtgaagagtttcacaaatccttcaaaataaaaagtaatgacttagaaactgccttcctgggtgatttgcatgtgtcttagtcttagtcaccttattatcctgacacaaaaacacatgagcatacatgtctacacatgactacacaaatgcaaacctttgcaaacacattatgcttttgcacacacacacctgtacacacacaccggcatgtttatacacagggagtgtatggttcctgtaagcactaagttagctgttttcatttaatgacctgtggtttaacccttttgatcactaccaccattatcagcaccagactgagcagctatatccttttattaatcatggtcattcattcattcattcattcacaaaatatttatgatgtatttactctgcaccaggtcccatgccaagcactggggacacagttatggcaaagtagacaaagcatttgttcatttggagcttagagtccaggaggaatacattagataatgacacaatcaaatataaattgcaagatgtcacaggtgtgatgaagggagagtaggagagaccatgagtatgtgtaacaggaggacacagcattattctagtgctgtactgttccgtacggcagccactacccacatgtaactttttaagatttaaatttaaattagttaacattcaaaacgcagctccccaatcacactagcaacatttcaagtgcttgagagccatgcatgattagtggttaccctattgaataggtcagaagtagaatcttttcatcatcacagaaagttctattggacagtgctcttctagatcatcataagactacagagcacttttcaaagctcatgcatgttcatcatgttagtgtcgtattttgagctggggttttgagactccccttagagatagagaaacagacccaagaaatgtgctcaattgcaatgggccacatacctagatctccagatgtcatttcccctctcttattttaagttatgttaagattactaaaacaataaaagctcctaaaaaatcaaactgtattctggtgttctcttctacacagtgggagggcgagcagtaggagagattggcccatttggtgctggccatttgaggaatgcaagcccagcactagtctcataatctctaggaatctgtagagagaggaattgaagtaaatttcagcattggctcattcagtcattcggcgacattcatcaggtacctgcaatgtgttaggggatcttatgagtaggcagcgtgcgtgatccttgctcccctggagctttctaacattctagcaggcagaccacacataaatttgcaatactgtttctgataaaaacgtgctgtaaaggaaataaagcagagaactatcatggaaaaaaaaaaaaaaaaaa

SEQ ID NO: 20 - RNA, 2989 nt, Homo sapiens
  source 1..2989: mol_type = mRNA
gtctgggccgagcccgcccagctggctgagacgcgtggagcctggcggcgagtgggggcgtgcgacggttactctggttactggggccgcgccgcgctggcgagagccgccgcccgcgagggatgctggtgaggaagccgtcgggagccgccgccgccatctgagggaggtaccctggaaaccaccttttatcggtggggaagtgcagtcgcggtgggcggctctgggggccagcgaaacgggaggcctctaaatctttaggttggggctgcattgccctggagccgcactcttgagtccgaggccatcttttgttggagaaggcgtcggcgttggcgttttcccgaggttgggctgtacagtgtctccgtccgcggaaaaagaagcctctgaacccgcgccggcccgcagcccccgtgccttccggccgctgctcgccgtcgccagaggctaggccacgtttcccccagtgccgaggtgtttctgtgaccctccctccactcccattcccttctgaaagggcacctgctcttggtgagaaaagaaattatagcacgaagagccagtatcagaagagtatccatcacccgcagcaaccgctcagggaacaccatcaaaaaagaaaaaaagggaatatctggatttcctgggcgaggaggagcgagtctgctcgggagctgttccagcaggcgatttttaaatactgctttctacgccctatacaacttggcttcacatacttttacactaactttatatgatttttaaaaactggtctgatcggacttctcgtcctgggacactgtttactggagtctggccggctctccgtgctcctcttggtacctcattttggggagaaccttaaacccactcgagcagataatctccgccttgaccggtgccaccaaagaagccttggaaccatgtggacttttctgggcattgccactttcacctatttttataagaagttcggggacttcatcactttggccaacagggaggtcctgttgtgcgtgctggtgttcctctcgctgggcctggtgctctcctaccgctgtcgccaccgaaacgggggtctcctcgggcgccagcagagcggctcccagttcgccctcttctcggatattctctcaggcctgcctttcattggcttcttctgggccaaatccccccctgaatcagaaaataaggagcagctcgaggccaggaggcgcagaaaaggaaccaatatttcagaaacaagcttaataggaacagctgcctgtacatcaacatcttctcagaatgacccagaagttatcatcgtgggagctggcgtgcttggctctgctttggcagctgtgctttccagagatggaagaaaggtgacagtcattgagagagacttaaaagagcctgacagaatagttggagaattcctgcagccgggtggttatcatgttctcaaagaccttggtcttggagatacagtggaaggtcttgatgcccaggttgtaaatggttacatgattcatgatcaggaaagcaaatcagaggttcagattccttaccctctgtcagaaaacaatcaagtgcagagtggaagagctttccatcacggaagattcatcatgagtctccggaaagcagctatggcagagcccaatgcaaagtttattgaaggtgttgtgttacagttattagaggaagatgatgttgtgatgggagttcagtacaaggataaagagactggagatatcaaggaactccatgctccactgactgttgttgcagatgggcttttctccaagttcaggaaaagcctggtctccaataaagtttctgtatcatctcattttgttggctttcttatgaagaatgcaccacagtttaaagcaaatcatgctgaacttattttagctaacccgagtccagttctcatctaccagatttcatccagtgaaactcgagtacttgttgacattagaggagaaatgccaaggaatttaagagaatacatggttgaaaaaatttacccacaaatacctgatcacctgaaagaaccattcttagaagccactgacaattctcatctgaggtccatgccagcaagcttccttcctccttcatcagtgaagaaacgaggtgttcttcttttgggagacgcatataatatgaggcatccacttactggtggaggaatgactgttgcttttaaagatataaaactatggagaaaactgctaaagggtatccctgacctttatgatgatgcagctattttcgaggccaaaaaatcattttactgggcaagaaaaacatctcattcctttgtcgtgaatatccttgctcaggctctttatgaattattttctgccacagatgattccctgcatcaactaagaaaagcctgttttctttatttcaaacttggtggcgaatgtgttgcgggtcctgttgggctgctttctgtattgtctcctaaccctctagttttaattggacacttctttgctgttgcaatctatgccgtgtatttttgctttaagtcagaaccttggattacaaaacctcgagcccttctcagtagtggtgctgtattgtacaaagcgtgttctgtaatatttcctctaatttactcagaaatgaagtatatggttcattaagcttaaaggggaaccatttgtgaatgaatatttggaacttaccaagtcctaagagacttttggaagaggatatatatagcatagtaccataccacttataaagtggaaactcttggaccaagatttggattaatttgtttttgaagttttttgtatataaatatgtaaatacatgctttaatttgcaatttaaaatgaaggggttaaataagttagacatttaaaagaaatgattgttaccataaattagtgctaatgctgaggagaactacagtttttcttttgaatttagtatttgagatgagttgttgggacatgcaaataaaatgaagaatgaa

SEQ ID NO: 21 - RNA, 3138 nt, Homo sapiens
  source 1..3138: mol_type = mRNA
attttacccagcccctgttcaagatggagttgctgtggttcacacatctctgacaaaaatacagggctattcggagtcaccagacctggagtttgagtatgctgacacagacaagtgggctgcagagctctcggagctttacagctacacggaagggccagaattcctgatgaatcgaaaatgctttgaggaggacttccggatccatgtgacagacaagaagtggactgagctggataccaaccagcaccggacccatgccatgaggctcctggatggcttggaagtcactgccagggagaagagactcaaggtggctcgagcaattctctatgttgctcaaggcacgtttggggagtgcagctcggaggcagaggtgcagtcctggatgcgctacaacatctttctcctcctggaggtgggcacgttcaatgctttggtggagcttctgaacatggaaatagacaacagtgccgcctgcagcagtgctgtgaggaagcctgccatctccctggctgacagcacagacctcagggtcctgctcaacatcatgtacctgatagtggagaccgttcatcaggagtgtgagggtgacaaggctgagtggaggaccatgcggcagaccttcagagccgagctgggctccccgctgtacaacaatgagccatttgccatcatgctgtttgggatggtgaccaaattttgcagtggtcacgcccctcactttcccatgaagaaagttctcttgctgctctggaagacagtattgtgcacgctaggcggctttgaggagctgcagagcatgaaggctgagaagcgcagcatcctgggcctccccccgcttcctgaggacagcatcaaagtgattcgcaacatgagagcagcctctccaccagcatctgcttcagacttgattgagcagcagcagaaacggggccgccgagagcacaaggctctgataaagcaggacaacctagatgccttcaacgagcgggatccctacaaggctgatgactctcgagaagaggaagaggagaatgatgatgacaacagtctggagggggagacgtttcccctggaacgggatgaagtgatgcctcccccgctacagcacccacagactgacaggctgacttgccccaaagggctcccgtgggctcccaaggtcagagagaaagacattgagatgttccttgagtccagccgcagcaaatttataggttacactctaggcagtgacacgaacacagtggtggggctgcccaggccaatccacgaaagcatcaagactctgaaacagcacaagtacacgtcgattgcagaggtccaggcacagatggaggaggaatacctccgctcccctctctcagggggagaagaagaagttgagcaagtccctgcagaaaccctctaccaaggcttgctccccagcctgcctcagtatatgattgccctcctgaagatcctgttggctgcagcacccacctcaaaagccaaaacagactcaatcaacatcctagcggacgtcttgcctgaggagatgcccaccacagtgttgcagagcatgaagctgggggtggatgtaaaccgccacaaagaggtcattgttaaggccatttctgctgtcctgctgctgctgctcaagcactttaagttgaaccatgtctaccagtttgaatacatggcccagcacctggtgtttgccaactgcattcctttgatcctaaagttcttcaatcaaaacatcatgtcctacatcactgccaagaacagcatttctgtcctggattaccctcactgcgtggtgcatgagctgccagagctgacggcggagagtttggaagcaggtgacagtaaccaattttgctggaggaacctcttttcttgtatcaatctgcttcggatcttgaacaagctgacaaagtggaagcattcaaggacaatgatgctggtggtgttcaagtcagcccccatcttgaagcgggccctaaaggtgaaacaagccatgatgcagctctatgtgctgaagctgctcaaggtacagaccaaatacttggggcggcagtggcgaaagagcaacatgaagaccatgtctgccatctaccagaaggtgcggcatcggctgaacgacgactgggcatacggcaatgatcttgatgcccggccttgggacttccaggcagaggagtgtgcccttcgtgccaacattgaacgcttcaacgcccggcgctatgaccgggcccacagcaaccctgacttcctgccagtggacaactgcctgcagagtgtcctgggccaacgggtggacctccctgaggactttcagatgaactatgacctctggttagaaagggaggtcttctccaagcccatttcctgggaagagctgctgcagtgaggctgttggttaggggactgaaatggagagaaaagatgatctgaaggtacctgtgggactgtcctagttcattgctgcagtgctcccatcccccaccaggtggcagcacagccccactgtgtcttccgcagtctgtcctgggcttgggtgagcccagcttgacctccccttggttcccagggtcctgctccgaagcagtcatctctgcctgagatccattcttcctttacttcccccaccctcctctcttggatatggttggttttggctcatttcacaatcagcccaaggctgggaaagctggaatgggatgggaacccctccgccgtgcatctgaatttcaggggtcatgctgatgcctctcgagacatacaaatccttgctttgtcagcttgcaaaggaggagagtttaggattagggccagggccagaaagtcggtatcttggttgtgctctggggtgggggtggggtgtttctgatgttattccagcctcctgctacattatatccagaagtaattgcggaggctccttcagctgcctcagcactttgattttggacagggacaaggtaggaagagaagcttcccttaaccagaggggccatttttccttttggctttcgagggcctgtaaatatctatatataattctgtgtgtattctgtgtcatgttggggtttttaatgtgattgtgtattctgtttacattaaaaagaagcaaaaataaaaaaaaaaaaa

SEQ ID NO: 22 - RNA, 3098 nt, Homo sapiens
  source 1..3098: mol_type = mRNA
gcacgcagccttagctccttccccgcccttgctcttcccagtttctccgtcagcctgcgggtcccggctggcggctgcttccggtaggagagcggtgtagagcgagcaggtctcagctcctcgtcatgtcatacggtcccttagacatgtaccggaacccggggccctcggggccccagctccgggacttcagcagcatcatccagacgtgcagcggcaacatccagcggatcagccaagccactgctcagataaagaatttgatgagccagctaggaactaagcaggactcaagcaagctacaggaaaatctgcaacagttacaacactccacaaatcagctcgccaaggaaacaaatgaattgctgaaagaattagggtccttgccccttcccttatctacttcagaacagcgccagcagagacttcagaaggaacgcctcatgaatgacttctctgcagccttaaacaatttccaggctgtgcagagaagggtatctgaaaaggaaaaggagagtattgccagagcaagagctggatctcgtctttctgcagaagagaggcaaagagaggagcagctggtctcatttgacagccatgaggagtggaaccagatgcagagccaggaggatgaggtggccatcactgagcaggatttggaacttattaaagaaagagaaacggcaattcggcagctggaggctgacattttggatgtcaatcagatatttaaagatttggccatgatgatccatgaccagggtgatctgattgatagcatagaagccaatgtggaaagctcagaggtgcacgtcgaaagagccactgaacagttacagcgagctgcttactatcagaaaaaatctcgcaagaagatgtgtatcctggtgcttgtcctgtcagtgattattctaatcttgggacttattatctggctagtttataaaacgaagtgattgcctccgatcgttctcccgctgagctgttttcaagggcaagtgcttgttgaagtcttgccagaacaaactgatcacaagaagacagcatatatcagaacgtcctgtaatcatttagttagaaactaactactaactagtctttggaattcgtgacctatggagacagtaattatcaatttattgattctattgatttctcaaattaggaattaacttatgtggattttgcttcctcttgtattctgattgcccttcatcccaagtgtttactgaaaattccattctagatattcttgttttgacaaatgacactacagtctcgtaatattgtcttttatgtatatacaaaatttacctttttactagcatctgagatagagttactttctggtacccagtatattggagtctgtcagaaactctataataggccaccagtttttattatttaacatttttatttgaatttctaagaagcctattctctatctattttgaaagattttggcactatatttaattggaaggtaaaatattgtacatgtgatccagagtaaatgagaagtctctatctgagctggtcagttactggagtacatgttactaatctgggtttaaagtttacttcattatctgctagtgtcatccacagcagttcatcctcatccacactaagccatcctgttagcttttaaaggaagttaatttaattaacattaatatactctatgggctccctctcccacctgtctgcatagaaaggcagaattagacatagcatgctttggaaaagcaaataggaattgttgggaatgatttaatcttgttgttgttgttgttgttgttcacttgtggttctacattcctggtgaatgatgaatgttgctgtcaaagggctgccccctaccttataagggttgctgggcatttgaaggcaggaagatttttaaagatagattgaggttggtttaaaattattcctgtaaaccaacaataaagcaaagaagaggttcatttttgtaaataacactggtttcaaatagtgatgttagacttaacctaatttataaacaagagattaatatctccatgcatagttttagacaaaaaaagatgtttcaataaaattactgtcttgtaatataaatgttgtccacttcccttttccacaggcctagaacagttaaagggaacataatttgtttaggctcccacataaatgtgaatctggccaacaactttggttcatcctttagtgaattagaggatttggctaccctgagtatatttatattcatttcttctgttctccttctgttattatacttaatcttctaaactaaactaatgtgaacagtagggaagcaagggcccaaatgcataagtttctttgcactgttgcacttacttaatacaaataaatgttttttaaagcttttgtagtatgtttttatgagttaacatcctaatgtggtaggtattaggtaatgtgctgtcatgagaaaaattgagacttccaagaaaactggacaccaggtgagggttggtttggagacggaataggtgtagctgcctttccttgaaaaacagtgtgtagagatggctgagtgcaatggctcacacttgtaatcccaacactttggaaggctgaggcgggaggatcagtatatcggtacgtctgagcccaagagttcaagatcagcctgggcagtatagcaagaccccatctccattttttttttaatgattttttaattaaaaaaaagaacaacaggatagagctgttggggtggcacagtggcccaaagagcagcttcagagataattccttggttctatgatccctgtttaactccaaattacagtcggacttggatacatcatttgtaacattgtaggaaagaaaaaagtcttggttgtgaaaaacgatttgcatttgggtaaaataaagtgaccatgcttttgttctgtaatactgtgtgacctgtggttgttgtaatggtgatcatggagagcaaatatgaacttggcctggattttaaatggcctagaatttgtggtagttgccaaagaggttctcctaggtggtcttaataaacctattcacagaattctccttaaaaaaaaaaaaaaaaaaaa

SEQ ID NO: 23 - RNA, 4035 nt, Homo sapiens
  source 1..4035: mol_type = mRNA
acttaacaaccgaagtaacccgcaatgcggaagggcgaggggattgcgagtcaccgagtttcccgcgcggcttgagtcacggcctagaaagagagatgttggggttcccaggaccaggacagaggtggtagtgaactctcatgggcatccagagaaggtcaggccccttgctgacaggcctatctgtggggctactgctgctcttcagctgggtgacccttgtccagccaacctctctctcagctctggtccaccaccctcacttgtgccagaccacccgggatgtccatggccgtcactaccctggtttcttttgccctcgtctgtctgattctccagaggaagcctactgctgccacctgcaggctgcagggggctcctgctgcacccgggctgaatttgaggccctgtaccaagtcaatctgtccgctcttccgcccccgcccatcctcaggggcccaggcccgctcctagtgctgggcctctacaacctactggttgtgaccctgatgaccgtagacctcgtgcacttctgctgcggtcggggccggagtctgggctggagccaccgcaggcctccctctgggtcctccgccgcgagctccctgcaggtctctgcggggacagcttaggtgcgcccggagcttgcctgcacctgcgatccagagccaagcgccccgcccctgcccgggcgcgctccctccttagccctgcccctctctgaccccacctccgacgcaagagtggggcggggcagctgccggtggcgtcccgaacccagactcgccccgccccagagactgcgcctgcgcgggcacgagacaacctctccgcgatgactgccagctcagtggagcagctgcggaaggagggcaatgagctgttcaaatgtggagactacgggggcgccctggcggcctacactcaggccctgggtctggacgcgacgccccaggaccaggccgttctgcaccggaaccgggccgcctgccacctcaagctggaagattacgacaaagcagaaacagaggcatccaaagccattgaaaaggatggtggggatgtcaaagcactctaccggcggagccaagccctagagaagctgggccgcctggaccaggctgtccttgacctgcagagatgtgtgagcttggagcccaagaacaaagttttccaggaggccttgcggaacatcgggggccagattcaggagaaggtgcgatacatgtcctcgacggatgccaaagtggaacagatgtttcagatactgttggacccagaagagaagggcactgagaaaaagcaaaaggcttctcagaacctggtggtgctggccagggaggatgctggagcggagaagatcttccggagtaatggggttcagctcttgcaacgtttactggacatgggagagactgacctcatgctggcggctctgcgtacgctggttggcatttgctctgagcatcagtcacggacagtggcaaccctgagcatactgggaactcggcgagtagtctccatcctgggcgtggaaagccaggctgtgtccctggctgcctgccacctgctgcaggttatgtttgatgccctcaaggaaggtgtcaaaaaaggcttccgaggcaaagaaggtgccatcattgtggatcctgcccgggagctgaaggtcctcatcagtaacctcttagatctgctgacagaggtgggggtctctggccaaggccgagacaatgccctgaccctcctgattaaagcggtgccccggaagtctctcaaggaccccaacaacagcctcaccctctgggtcatcgaccaaggtctgaaaaagattttggaagtggggggctctctacaggaccctcctggggagctcgcagtgaccgcaaacagccgcatgagcgcctctattctcctcagcaagctctttgatgacctcaagtgtgatgcggagagggagaatttccacagactttgtgaaaactacatcaagagctggtttgagggccaagggctggccgggaagctacgggccatccagacggtgtcctgcctcctgcagggcccatgtgacgctggcaaccgggccttggagctgagcggtgtcatggagagtgtgattgctctgtgtgcctctgagcaggaggaggagcagctggtggccgtggaggctctgatccatgcagccggcaaggctaagcgggcctcattcatcactgccaatggtgtctcgctgctgaaggacctatataagtgcagcgagaaggacagcatccgcatccgggcgctagtgggactctgtaagctcggttcggctggagggactgacttcagcatgaagcagtttgctgaaggctccactctcaaactggctaagcagtgtcgaaagtggctgtgcaatgaccagatcgacgcaggcactcggcgctgggcagtggagggcctggcttacctgacctttgatgccgacgtgaaggaagagtttgtggaggatgcggctgctctgaaagctctgttccagctcagcaggttggaggagaggtcagtgctctttgcggtggcctcagcgctggtgaactgcaccaacagctatgactacgaggagcccgaccccaagatggtggagctggccaagtatgccaagcagcatgtgcccgagcagcaccccaaggacaagccaagcttcgtgcgggctcgggtgaagaagctgctggcagcgggtgtggtgtcggccatggtgtgcatggtgaagacggagagccctgtgctgaccagttcctgcagagagctgctctccagggtcttcttggctttagtggaagaggtagaggaccgaggcactgtggttgcccagggaggcggcagggcgctgatcccgctggccctggaaggcacggacgtggggcagacaaaggcagcccaggcccttgccaagctcaccatcacctccaacccggagatgaccttccctggcgagcggatctatgaggtggtccggcccctcgtctccctgttgcacctcaactgctcaggcctgcagaacttcgaggcgctcatggccctaacaaacctggctgggatcagcgagaggctccggcagaagatcctgaaggagaaggctgtgcccatgatagaaggctacatgtttgaggagcatgagatgatccgccgggcagccacggagtgcatgtgtaacttggccatgagcaaggaggtgcaggacctcttcgaagcccagggcaatgaccgactgaagctgctggtgctgtacagtggagaggatgatgagctgctacagcgggcagctgccgggggcttggccatgcttacctccatgcggcccacgctctgcagccgcattccccaagtgaccacacactggctggagatcctgcaggccctgcttctgagctccaaccaggagctgcagcaccggggtgctgtggtggtgctgaacatggtggaggcctcgagggagattgccagcaccctgatggagagtgagatgatggagatcttgtcagtgctagctaagggtgaccacagccctgtcacaagggctgctgcagcctgcctggacaaagcagtggaatatgggcttatccaacccaaccaagatggagagtgagggggttgtccctgggcccaaggctcatgcacacgctacctattgtggcacggagagtaaggacggaagcagctttggctggtggtggctggcatgcccaatactcttgcccatcctcgcttgctgccctaggatgtcctctgttctgagtcagcggccacgttcagtcacacagccctgcttggccagcactgcctgcagcctcactcagaggggccctttttctgtactactgtagtcagctgggaatggggaaggtgcatcccaacacagcctgtggatcctggggcatctggaagggcgcacacatcagcagcctcaccagctgtgagcctgctatcaggcctgcccctccaataaaagtgtgtagaactccaaaaaaaaaaaaaaaaaaaaaaaaaaaaaaaaaa

SEQ ID NO: 24 - RNA, 3729 nt, Homo sapiens
  source 1..3729: mol_type = mRNA
cgaaggggcgtggccaagcgcaccgcctcggggcggggccggcgttctagcgcatcgcggccgggtgcgtcactcgcgaagtggaatttgcccagacaagcaacatggctcggaaacgcgcggccggcggggagccgcggggacgcgaactgcgcagccagaaatccaaggccaagagcaaggcccggcgtgaggaggaggaggaggatgcctttgaagatgagaaacccccaaagaagagccttctctccaaagtttcacaaggaaagaggaaaagaggctgcagtcatcctgggggttcagcagatggtccagcaaaaaagaaagtggccaaggtgactgttaaatctgaaaacctcaaggttataaaggatgaagccctcagcgatggggatgacctcagggactttccaagtgacctcaagaaggcacaccatctgaagagaggggctaccatgaatgaagacagcaatgaagaagaggaagaaagtgaaaatgattgggaagaggttgaagaacttagtgagcctgtgctgggtgacgtgagagaaagtacagccttctctcgatctcttctgcctgtgaagccagtggagatagagattgaaacgccagagcaggcgaagacaagagaaagaagtgaaaagataaaactggagtttgagacatatcttcggagggcgatgaaacgtttcaataaaggggtccatgaggacacacacaaggttcaccttctctgcctgctagcaaatggcttctatcgaaataacatctgcagccagccagatctgcatgctattggcctgtccatcatcccagcccgctttaccagagtgctgcctcgagatgtggacacctactacctctcaaacctggtgaagtggttcattggaacatttacagttaatgcagaactttcagccagtgaacaagataacctgcagactacattggaaaggagatttgctatttactctgctcgagatgatgaggaattggtccatatattcttactgattctccgggctctgcagctcttgacccggctggtattgtctctacagccaattcctctgaagtcagcaacagcaaagggaaagaaaccttccaaggaaagattgactgcggatccaggaggctcctcagaaacttccagccaagttctagaaaaccacaccaaaccaaagaccagcaaaggaaccaaacaagaggaaacctttgctaagggcacctgcaggccaagtgccaaagggaagaggaacaagggaggcagaaagaaacggagcaagccctcctccagcgaggaagatgagggcccaggagacaagcaggagaaggcaacccagcgacgtccgcatggccgggagcggcgggtggcctccagggtgtcttataaagaggagagtgggagtgatgaggctggcagcggctctgattttgagctctccagtggagaagcctctgatccctctgatgaggattccgaacctggccctccaaagcagaggaaagcccccgctcctcagaggacaaaggctgggtccaagagtgcctccaggacccatcgtgggagccatcgtaaggacccaagcttgccagcggcatcctcaagctcttcaagcagtaaaagaggcaagaaaatgtgcagcgatggtgagaaggcagaaaaaagaagcatagctggtatagaccagtggctagaggtgttctgtgagcaggaggaaaagtgggtatgtgtagactgtgtgcacggtgtggtgggccagcctctgacctgttacaagtacgccaccaagcccatgacctatgtggtgggcattgacagtgacggctgggtccgagatgtcacacagaggtacgacccagtctggatgacagtgacccgcaagtgccgggttgatgctgagtggtgggccgagaccttgagaccataccagagcccatttatggacagggagaagaaagaagacttggagtttcaggcaaaacacatggaccagcctttgcccactgccattggcttatataagaaccaccctctgtatgccctgaagcggcatctcctgaaatatgaggccatctatcccgagacagctgccatccttgggtattgtcgtggagaagcggtctactccagggattgtgtgcacactctgcattccagggacacgtggctgaagaaagcaagagtggtgaggcttggagaagtaccctacaagatggtgaaaggcttttctaaccgtgctcggaaagcccgacttgctgagccccagctgcgggaagaaaatgacctgggcctgtttggctactggcagacagaggagtatcagcccccagtggccgtggacgggaaggtgccccggaacgagtttgggaatgtgtacctcttcctgcccagcatgatgcctattggctgtgtccagctgaacctgcccaatctacaccgcgtggcccgcaagctggacatcgactgtgtccaggccatcactggctttgatttccatggcggctactcccatcccgtgactgatggatacatcgtctgcgaggaattcaaagacgtgctcctgactgcctgggaaaatgagcaggcagtcattgaaaggaaggagaaggagaaaaaggagaagcgggctctagggaactggaagttgctggccaaaggtctgctcatcagggagaggctgaagcgtcgctacgggcccaagagtgaggcagcagctccccacacagatgcaggaggtggactctcttctgatgaagaggaggggaccagctctcaagcagaagcggccaggatactggctgcctcctggcctcaaaaccgagaagatgaagaaaagcagaagctgaagggtgggcccaagaagaccaaaagggaaaagaaagcagcagcttcccacctgttcccatttgagcagctgtgagctgagcgcccactagaggggcacccaccagttgctgctgccccactacaggccccacacctgccctgggcatgcccagcccctggtggtgggggcttctctgctgagaaggcaaactgaggcagcatgcacggaggcggggtcaggggagacgaggccaagctgaggaggtgctgcaggtcccgtctggctccagcccttgtcagattcacccagggtgaagccttcaaagctttttgctaccaaagcccactcaccctttgagctacagaacactttgctaggagatactcttctgcctcctagacctgttctttccatctttagaaacatcagtttttgtatggaagccaccgggagatttctggatggtggtgcatccgtgaatgcgctgatcgtttcttccagttagagtcttcatctgtccgacaagttcactcgcctcggttgcggacctaggaccatttctctgcaggccacttaccttcccctgagtcaggcttactaatgctgccctcactgcctctttgcagtaggggagagagcagagaagtacaggtcatctgctgggatctagttttccaagtaacattttgtggtgacagaagcctaaaaaaagctaaaatcaggaaagaaaaggaaaaatacgaattgaaaattaaggaaatgttagtaaaatagatgagtgttaaactagattgtattcattactagataaaatgtataaagctctctgtactaaggagaaatgacttttataacattttgagaaaataataaagcatttatctaaaaaaaaaaa

SEQ ID NO: 25 - RNA, 2817 nt, Homo sapiens
  source 1..2817: mol_type = mRNA
agtcggcggccggactgggaagatggacgcagctactctgacctacgacactctccggtttgctgagtttgaagattttcctgagacctcagagcccgtttggatactgggtagaaaatacagcattttcacagaaaaggacgagatcttgtctgatgtggcatctagactttggtttacatacaggaaaaactttccagccattggggggacaggccccacctcggacacaggctggggctgcatgctgcggtgtggacagatgatctttgcccaagccctggtgtgccggcacctaggccgagattggaggtggacacaaaggaagaggcagccagacagctacttcagcgtcctcaacgcattcatcgacaggaaggacagttactactccattcaccagatagcgcaaatgggagttggcgaaggcaagtccataggccagtggtacgggcccaacactgtcgcccaggtcctgaagaagcttgctgtcttcgatacgtggagctccttggcggtccacattgcaatggacaacactgttgtgatggaggaaatcagaaggttgtgcaggaccagcgttccctgtgcaggcgccactgcgtttcctgcagattccgaccggcactgcaacggattccctgccggagctgaggtcaccaacaggccgtcgccatggagacccctggtacttctcattcccctgcgcctggggctcacggacatcaacgaggcctacgtggagacgctgaagcactgcttcatgatgccccagtccctgggcgtcatcggagggaagcccaacagcgcccactacttcatcggctacgttggtgaggagctcatctacctggacccccacaccacgcagccagccgtggagcccactgatggctgcttcatcccggacgagagcttccactgccagcacccgccgtgccgcatgagcatcgcggagcttgacccgtccatcgctgtggggtttttctgtaagactgaagatgacttcaatgattggtgccagcaagtcaaaaagctgtctctgcttggaggtgccctgcccatgtttgagctggtggagctgcagccttcacatctggcctgccccgacgtcctgaacctgtccctaggtgagagctgccaagtccagattcttctgatgtagagcgactggaaagattcttcgactcagaagatgaagactttgaaatcctgtccctttgaaaatcctggggtcgggggtggcacctgtgagagcctggggctcctggtgccgctgcgtttcatccatcccgcccgctcgcctgccgagggctgcgccccgtgctgcctccccccagagggccacccgctgtgctcgtggactgaggctgcgctgcccgggaggccttactgcttggtgtcagactgcccagctcagagtgcccgtcagggcctgtgcatccgcacgcggagccgtctgttaggagcttccagagtgttctctcgacactgccagccccgtgttagcacctgggcctcagtcccacttgctcccaggcgccggttctgtggttggtttggaattaaagtcctgtttgaagttgtcagacacagacatgaatttctgggcgctccctgagtcagagtctcagaagacctgtgcaggctggcgtgagaggagcggcagccacactgcggccccacgcccaaggactgggctgctctcgaggggggcgcgcccaccgctgtgtcctctctgcccagcctggcttaccaagggctacctcagtgggagatgaggttggaggaacgaaggcgaggttcctccttgctttggggagaaaagtattcaggaagtgggtgtgtgggaaacctgaagatggcgtgcacaggacacagcgtgggcggcctgggcagaagggcggctggctgtcctggagctgctgctggagcctgccctcagagtgtccctttccagtgctgtggcattctgtggcagcttccccaggtgtggtgacggggggggggcggggcctccacctgtgacagccaggcttgagggtggacggcgtgcctctcccaggagccttccccatgtccttgccttgctgagaattgccctcccatgccgctgaggtgttaggtggtttagggccaaaaggggaaaaccacttgagtcttgtggtgtgtggtgggcagacaccacagggtggcatcacctggtggcatttccagaacctcagccccgattccagcacccaccaccgcctgaccctgtgtaacctgctgtcccgggtcccagagtgcactctgccccgctgctctgctgcctgtcctgggaaagtatctttgccccactaggaaatgtaaacaggagggcttggggagcgtgggcacttttctcatgagcagctactgcggcgttggcaggactcgctgctgctgctgctgcttgtgtaggtcggggagccagagatccccgaggacgcgcgccggacagtcggcactgaccggcccacctggtagcagaggacacccccagccccccaagcattgaagacatagtgtatttcctcgtatcctttctcccttgggtgtagttggggtggggaagcagggaaggctggtgcgatctccattccttgggctccacgtccgagttcatggtgcgccgctgtgctgggagctgcagtggtaatgtgtgggacaccttgaccaaaggggagctttgtctcgtgtgttttgaaaaaggcttaatgaagagaatgttgttcattcttagtagtatagtttgcaattcttaatggcaaataataagtttcagtagaaaacaaa

SEQ ID NO: 26 - RNA, 1900 nt, Homo sapiens
  source 1..1900: mol_type = mRNA
gctctctcgcgctaccctcccgccgcccgcggtcctccgtcggttctctcgttagtccacggtctggtcttcagctacccgccttcgtctccgagtttgcgactcgcggaccggcgtccccggcgcgaagaggctggactcggattcgttgcctgagagatggtgtcttgctatgttgcccagacagatcttgtactcctgggctcaagttgtcctcctgcctcagcctcccaaagtgctgggattaaagatcaatggctgccatccggaagaaactggtgattgttggtgatggagcctgtggaaagacatgcttgctcatagtcttcagcaaggaccagttcccagaggtgtatgtgcccacagtgtttgagaactatgtggcagatatcgaggtggatggaaagcaggtagagttggctttgtgggacacagctgggcaggaagattatgatcgcctgaggcccctctcctacccagataccgatgttatactgatgtgtttttccatcgacagccctgatagtttagaaaacatcccagaaaagtggaccccagaagtcaagcatttctgtcccaacgtgcccatcatcctggttgggaataagaaggatcttcggaatgatgagcacacaaggcgggagctagccaagatgaagcaggagccggtgaaacctgaagaaggcagagatatggcaaacaggattggcgcttttgggtacatggagtgttcagcaaagaccaaagatggagtgagagaggtttttgaaatggctacgagagctgctctgcaagctagacgtgggaagaaaaaatctgggtgccttgtcttgtgaaaccttgctgcaagcacagcccttatgcggttaattttgaagtgctgtttattaatcttagtgtatgattactggcctttttcatttatctataatttacctaagattacaaatcagaagtcatcttgctaccagtatttagaagccaactatgattattaacgatgtccaacccgtctggcccaccagggtccttttgacactgctctaacagccctcctctgcactcccacctgacacaccaggcgctaattcaaggaatttcttaacttcttgcttctttctagaaagagaaacagttggtaacttttgtgaattaggctgtaactactttataactaacatgtcctgcctattatctgtcagctgcaaggtactctggtgagtcaccacttcagggctttactccgtaacagattttgttggcatagctctggggtgggcagttttttgaaaatgggctcaaccagaaaagcccaagttcatgcagctgtggcagagttacagttctgtggtttcatgttagttaccttatagttactgtgtaattagtgccacttaatgtatgttaccaaaaataaatatatctaccccagactagatgtagtattttttgtataattggatttcctaatactgtcatcctcaaagaaagtgtattggttttttaaaaaagaaagtgtatttggaaataaagtcagatggaaaattcattttttaaattcccgttttgtcactttttctgataaaagatggccatattaccccttttcggccccatgtatctcagtaccccatggagctgggctaagtaaataggaattggtttcacgcctgaggcaattagacactttggaagatggcataacctgtctcacctggacttaagcatctggctctaattcacagtgctcttttctcctcactgtatccaggttccctcccagaggagccaccagttctcatgggtggcactcagtctctcttctctccagctgactaaactttttttctgtaccagttaatttttccaactactaatagaataaaggcagttttctaaa

SEQ ID NO: 27 - RNA, 4445 nt, Homo sapiens
  source 1..4445: mol_type = mRNA
cttgcggaagtgacggcagttccgagtccagtgggggcggtgggagcgatgagggtctgagacggtgggagcggttgtgtgaagatggagacattccatacaccaagcttgggtgatgaggaatttgaaatcccacctatctccttggattctgatccctcattggctgtctcagatgtggttggccactttgatgacctggcagacccttcctcttcacaggatggcagtttttcagcccagtatggggtccagacattggacatgcctgtgggcatgacccatggcttgatggagcagggcggggggctcctgagtgggggcttgaccatggacttggaccactctataggaactcagtatagtgccaacccacctgttacaattgatgtaccaatgacagacatgacatctggcttgatggggcatagccagttgaccaccattgatcagtcagaactgagttcccagctgggtttgagcctagggggtggcaccatcctgccacctgcccagtcacctgaagatcgtctttcaaccaccccttcacctactagttcacttcacgaggatggtgttgaggatttccggaggcaacttcccagccagaagacagtcgtggtggaagcagggaaaaagcagaaggccccaaagaagagaaaaaagaaagatcctaatgaacctcagaaaccagtttcagcatatgctttattctttcgtgatacacaggctgccatcaagggacagaatcctaatgccacttttggtgaggtttcaaaaattgtggcctccatgtgggatagtcttggagaggagcaaaaacaggtatataagaggaaaactgaggctgccaagaaagagtatctgaaggcactggctgcttacaaagacaaccaggagtgtcaggccactgtggaaacagtggaattggatccagcaccaccatcacaaactccttctccacctcctatggctactgttgacccagcatctccagcaccagcttcaatagagccccctgccctgtccccatccattgttgttaactccaccctttcatcctatgtggcaaaccaggcatcttctggagctgggggtcagcccaatatcaccaagttgattattaccaaacaaatgttgccctcttctattactatgtctcaaggagggatggttactgttatcccagccacagtggtgacctcccgggggctccaactaggccaaaccagtacagctactatccagcccagtcaacaagcccagattgtcactcggtcagtgttgcaggcagcagcagctgctgctgctgctgcttctatgcaactgcctccaccccgactacagccccctccattacaacagatgccacagcccccgactcagcagcaagttaccattctgcagcagcctcctccactccaggccatgcaacagcctccacctcagaaagttcgaatcaatttacagcaacagcctcctcctctgcagatcaagagtgtgcctctacccactttgaaaatgcagactaccttagtcccaccaactgtggaaagtagtcctgagcggcctatgaacaacagccctgaggcccatacagtggaggcaccttctcctgagactatctgtgagatgatcacagatgtagttcctgaggttgagtctccttctcagatggatgttgaattggtgagtgggtctcctgtggcactctcaccccagcctcgatgtgtgaggtctggttgtgagaaccctcccattgtgagtaaggactgggacaatgaatactgcagcaatgagtgtgtggtgaagcactgcagggatgtattcttggcctgggtagcctctagaaattcaaacacagtggtgtttgtgaaatagtccttcctgttctccaagccagtgaagagttatctgctgggaaagtgtccaagagcctgtttttgaaacacaagctgggcttctggtagtgcctcatcacaacccatgatggctgttcatgtttcaccccttttcttccttcagcagaggccaggctatggagcagggccactgaatttgctgtaatctggagatgctttttactttcaaccataagcggtaatagcagaggaaagggtgaagggagtctgggcaagcaaagcatagagatggtggggtggtggtggggttgaagaaacttgttggtataattgtcataggacttgcctaaaatattattaaaattacgggagtgtactcagctttgagcctaggagaaaatgccactgtgtgcatccattttaaagggttccctcataaaaaaatgttattccccattatcacatcagtacactgctttgaaaacaaaacttttcaacatgggcatactgggctacatggaaaatgacatcacccaggagtgatttctctttatatatattatttctgcagttaccatccttatctgagttatcacagttcatgaatctaagaggcggaactctacatcattagtaagaggttccaccaaagtctaaagttgtattcacttgtgtttgatgaactatctttaaaagaccataggtctatcattatttcttagacataatctaaagaaaaacagactagagaagccacctggttgtaacagaataagcagaagtttacagcatgatagtccaagtggtgataactttaaataaaactcaaatttttactgtttgtagacaggaatgctgtcctagagaacctcctcctcaaccagctacgtacatagttttatcctatgcattcctgttttctgtgtgttttttgttttttttttttttttttttttttgagacagagtctcgctctgtcacccaggctggagtgcagtggtgcgacctcagctcactgaaacctctgcctcccgggttcaagcgattctcctgcatcagcctcccgagtagctaggattacaggcgcccgccactacgcccagctaatttgtggtatttttagtagagacagggtttcaccatgttggccaggctggtctcgaactcctgacctcatgatccgcccgccttgacctcccaaagtgctgggattacaggcatgagccaccgcacccagcctgcattcctgtttttttaatggttttggagggtagcagtagagatggggtctcactatgttgcccagtctagtcttgaactcctgggctacagttaccctcctacctcggcttcccaaagtgctcggattacaggtgtgagccactgtgcctagcctataatgatcattttaatgtttcccatgcactcatttagtttgaaccttcacagcaacccaatgaggtaatactcccatttcacatataatactgagagatgagttgcacaagattatacactgttaagtagcagagccagaatggacttcagaatcccaactacaatacaaatgtttatttaaataaagaagaaagctattgtacaaatatcactcttcaggtttagcttacagagccatggctatggattcttagctctgtaaggaagtgcttctataaattcttaggtttagagatgataccatctgggtacctttgcttgaaccgtgcaaccacatctgggtctagtaggtggatcccatccagttggtttccaagggtgatcctgaaacagtgtaaaaggaggggcaaaccagaaatcctggaattagagggtttaatattgttaaaaaatgcataccaaatgaagactgcctatcatcatatcaaatatgccaattctaaaaagagcttaacattagaatagtatatggtagaattactagttcagaattggcatagattctggtgttaaaatagactggatctgtattatctgagggttagtaactaatgcttagccaggcctgcttcacagagttgctaccagggagtattctttggataagcaaaatgctagcagcatgtgttttaagctctgttaaggggtgaaagatgtaattattgacagattaaatagataacttcgtaaccaccagggggcagattcaatacatcacagaatggctgaggaagatccttgggttgtgaagagagtagaaaccctagggagcagtgcttttgggtcctagaacctgttgagtttctaatgaatatttgtagaatctcataaaacagtttaaatacaagcttaagtggcttatgaatcctgtgaagctcatttatggactagtgtaaaacaatgtgaagctctactaagttctgtccttaatcataaataatagccccttgaggactagcctgttctctggtcaccttaccagttgggttgcacattgtgtggtcgtccaaataactcaatcttgcgagtgccaggagatagtctttcaatcatgccatagatttcatctggtttatgactggtggaacgaacctaggaaataaaaactagctgctttttaagtta

SEQ ID NO: 28 - RNA, 4713 nt, Homo sapiens
  source 1..4713: mol_type = mRNA
cttttccgcccgctcccccctccccccgagcgccgctccggctgcaccgcgctcgctccgagtttcaggctcgtgctaagctagcgccgtcgtcgtctcccttcagtcgccatcatgattatctaccgggacctcatcagccacgatgagatgttctccgacatctacaagatccgggagatcgcggacgggttgtgcctggaggtggaggggaagatggtcagtaggacagaaggtaacattgatgactcgctcattggtggaaatgcctccgctgaaggccccgagggcgaaggtaccgaaagcacagtaatcactggtgtcgatattgtcatgaaccatcacctgcaggaaacaagtttcacaaaagaagcctacaagaagtacatcaaagattacatgaaatcaatcaaagggaaacttgaagaacagagaccagaaagagtaaaaccttttatgacaggggctgcagaacaaatcaagcacatccttgctaatttcaaaaactaccagttctttattggtgaaaacatgaatccagatggcatggttgctctattggactaccgtgaggatggtgtgaccccatatatgattttctttaaggatggtttagaaatggaaaaatgtgatgcaaaagaaagaaatccctgcgctttctgtctgtctttgtggcggcccagattgaattggggaatacatctttagcctggaaatgtaggctgcatgttaatggtaatgtaacttttgcagtgtaatgtttgaaaaatattaatgtagtttttgcttttacagtaacaaatgtggcaattattttggatctatcacctgtcatcataactggcttctgcttgtcatccacacaacaccaggacttaagacaaatgggactgatgtcatcttgagctcttcatttattttgactgtgatttatttggagtggaggcattgtttttaagaaaaacatgtcatgtaggttgtctaaaaataaaatgcatttaaactcatttgagagaatgccttttagtttaatgcatatttaaactaaattgatcctgtagtgttcctggagaagctagagcctgattgtaggctactactcatcaattaacttctacagtggagactacttctgggactggaatataaaaaagaatcaaaggttctgattttgagttgcaataaagggaaagaccatgctcatagcagtgccaacatctgaagtgtggagccttacccatttcatcacctacaacggaagtagttaactggaagagattaccaagagaataaaaagagactcattcagtggaagcaactttgtctcagcttatttcacataaagagagcgaagtcttttgggatgaatgttaattaaactccctggtaactagaacagggactggcaaactagcctatctgaccacctgttttgtacactttaaggtggttggttgcctttttaaatggttgaggggaaaagaataccttgtgggatatggaatttaagttcgagtccagttttattggaacgtggctatgcttattcatttatggattgactgtggctgttgtcagtgcatgagcagagttgtgtctaacagactagagcctgcaagtttgccagcccctgatttaaaagatgaaggtacacagaatgtgggctggctggtgggcaaaggggtaaaaatgttctctatattgtatctgaaaagatggggtgtctgaataagaaaatgcatctatttgacagacctggagcagttgctatctgctgctatggtttccaccacagatgcaagaagaacatgtccttgcgctttccgtctgtctaattgtggcagctgagattgaatagaggaatacaggaggaaaaaaagcgggaagagtttttgaggcaggtcggtcacccaggcttgtagtgcagtggcacaagcaactcactgcattctctgcatcctgtgctcaagccattttcccacctcagtctcactagttgctgggactgcaggcatgcacccctatgcccagctaatttttgtagagaccgagtatcgcttagttgcccagggtggtctcaactcctgggctcaaggagatctgcccacctcagcctcccaaagtgcaggcctagcctgggaggggaattttcaaaacgtgagttttgggaaatagtctatcagccttacctggttgattacacttgtaaaagaaagattaaaagcaggccagtgactctggtctgcttgaacatgtgaatgtagtggtttgagcaatctggagtttgccctagtgtcaaattccagactgtccatagtgtccaaaacctgaggcagatactaatgttaacccccagcaccccgtgattggaaacaaacctaaatacgtattgggaacttaatagcaattttaagcattctgatagattttttgtagggatggggtcatgccatgtggcccaggctggtctgaaaactctggcctcaagtgatctcaagctttggccttctaaagtgttgggattacaggtgtgaggcattgcacctggcttagcgttctgatttgacattgtaatgaaaagtgtgagtctcatctacagggccttttgtcctctgaaatgatagcaggaagggaattttcaggcagtggtcaaagctggggaaaccaggatagtgaagaaggccttgaggtgagagatggaagctaattggtgaactagccttggaagcctgaaacagacaagtagcaattcagagactttgtgggctccactgctccaacttgttttgaagattttcagttctgcagaagaggtatttccccagttgtcctttcagtgctcttagctgttttcccaacatccagatccaatcaaggctgggacatagcattttatcatgtctatttaagtcagaagtgatgaaccccagctgtttacctcatggtaaacctttgaagattccaggtagaatcttctcagactttgaagactgtctcattttatatctttttctcgttattcctagggtcaagacgttttgggcaagaataaggatgtgaacatcagaaagctcataacattttgtttttgatgctaagtttaacaaaggcatgctttagtagcctgtgggccctagggtttgttaaagtgtggagaacaactgagtggagcaagaggacttttctaggaaggtccttgtaatgtgacatttgaaaacaaatgaaggtgtggaagtaggccatgtggatatcaggacaaaccattccaggccaagacaacagcagttagtctggagtgtgatgtgttctgggaaaaaagtggccactttgctaacccaagaagacaggaagggttgtaaagcagtgggagtgtgcaaggaaggaagaccagacctcaaggaaaccacaggcgctctgagcagaagagttacatgatatgactcaaatttttaaaggatcactttggctgccaggtggcagggtaaaagcatagaataattgtgtataatgtgtttttaaggcaaagatagtggcttagtctagggtagtagactgaggtggtaggaaatgaagatagagacaacaggatatgctggtgggtgaggatggatttaatgttgatacaagtattttggtctgagcgtttggaagaaagttggcactgaggtgggaagtcgagtttagttttgttagttttggatgtgttaagtttgagatgctgattcttcagagaagtctaagctggagaactatatagagagtggaaagataacaatagacattgaaagccatgatacaggataaggtcatttggagagaggatagactgcattccaacatgagattggttgacaaagagaaaccaacaaaggtaattaagaggtgctcccactgcacttgtactcagaaggctgaggtaggattgttagaggccagcctgggcaccacagggagaccccatctctaaaatttagccaggaaccatggctcatgcctgtagccccaggaatttgggaggctgagtggggaggatcgcttgaggtcaggagtttgagaccagcctgggcaacatagggagacctaaaaaaattaattgggcatctgtagtcccagctactcaggcggctgagctgagaggatggcttgagtccgagagattgagggtgcagtgagctgtgatcataccactgcactccagcctgggcggcagtgagacactatctgaaaaaagtttaaaaattttaaaaaagaaggaactgcccctgaggtaagaaccaagggagggcctcccagaggtcaggtggaaaaagttttaggaaggaggaagtagtcaacagggttacctgttgcaaagtacttaagtaatatgaggcctgatagtggtaaacttgactaccgttggatttcactagtgggaaaggaagtctaattaaaatgcactcaagagactaacagtcgcaggcatgaaatacaatacaggtacatggttttttattatgtgtgcatctgcttcagtaataggtgtgaattactcatttggatcattaggagtttcaaaatctagttaaatgactagatttttgttgatgtaaattctgtcattctgaactgcagggattgtcagtaacttaactgcaaactaaactggtgataattatggtaaaattgcaagacgagcaataaatctcaaccaacttgagagaacactgataa

SEQ ID NO: 29 - RNA, 2610 nt, Homo sapiens
  source 1..2610: mol_type = mRNA
attctttgtctacttcctatttgagatgttagtagagaactgtcctgggtgaatctttcagtattgcagggcttggcaacttgctgcccgacaaaatacatcagaatttctctttaagaacaatatgggatggattaaaaaatatatatatgggatgaaattgggggtacttcaataccttgcatgccacccaagcattccttatcacacagatgcattttaagtgtaacagcaagcctaatggctactcgattttctttcccttcaggtgagaatgagatggtgatcatgagacctggaaacaaatatgagtacaagttcggctttgagcttcctcaggggcctctgggaacatccttcaaaggaaaatatgggtgtgtagactactgggtgaaggcttttcttgaccgcccgagccagccaactcaagagacaaagaaaaactttgaagtagtggatctggtggatgtcaatacccctgatttaatggcacctgtgtctgctaaaaaagaaaagaaagtttcctgcatgttcattcctgatgggcgggtgtctgtctctgctcgaattgacagaaaaggattctgtgaaggtgatgagatttccatccatgctgactttgagaatacatgttcccgaattgtggtccccaaagctgccattgtggcccgccacacttaccttgccaatggccagaccaaggtgctgactcagaagttgtcatcagtcagaggcaatcatattatctcagggacatgcgcatcatggcgtggcaagagccttcgggttcagaagatcaggccttctatcctgggctgcaacatccttcgagttgaatattccttactgatctatgttagcgttcctggatccaagaaggtcatccttgacctgcccctggtaattggcagcagatcaggtctaagcagcagaacatccagcatggccagccgaaccagctctgagatgagttgggtagatctgaacatccctgataccccagaagctcctccctgctatatggatgtcattcctgaagatcaccgattggagagcccaaccactcctctgctagatgacatggatggctctcaagacagccctatctttatgtatgcccctgagttcaagttcatgccaccaccgacttatactgaggtggatccctgcatcctcaacaacaatgtgcagtgagcatgtggaagaaaagaagcagctttacctacttgtttctttttgtctctcttcctggacactcactttttcagagactcaacagtctctgcaatggagtgtgggtccaccttagcctctgacttcctaatgtaggaggtggtcagcaggcaatctcctgggccttaaaggatgcggactcatcctcagccagcgcccatgttgtgatacaggggtgtttgttggatgggtttaaaaataactagaaaaactcaggcccatccattttctcagatctccttgaaaattgaggccttttcgatagtttcgggtcaggtaaaaatggcctcctggcgtaagcttttcaaggttttttggaggctttttgtaaattgtgataggaactttggaccttgaacttacgtatcatgtggagaagagccaatttaacaaactaggaagatgaaaagggaaattgtggccaaaactttgggaaaaggaggttcttaaaatcagtgtttcccctttgtgcacttgtagaaaaaaaagaaaaaccttctagagctgatttgatggacaatggagagagctttccctgtgattataaaaaaggaagctagctgctctacggtcatctttgcttagagtatactttaacctggcttttaaagcagtagtaactgccccaccaaaggtcttaaaagccatttttggagcctattgcactgtgttctcctactgcaaatattttcatatgggaggatggttttctcttcatgtaagtccttggaattgattctaaggtgatgttcttagcactttaattcctgtcaaattttttgttctccccttctgccatcttaaatgtaagctgaaactggtctactgtgtctctagggttaagccaaaagacaaaaaaaattttactacttttgagattgccccaatgtacagaattatataattctaacgcttaaatcatgtgaaagggttgctgctgtcagccttgcccactgtgacttcaaacccaaggaggaactcttgatcaagatgcccaaccctgtgatcagaacctccaaatactgccatgagaaactagagggcaggtcttcataaaagccctttgaacccccttcctgccctgtgttaggagatagggatattggcccctcactgcagctgccagcacttggtcagtcactctcagccatagcactttgttcactgtcctgtgtcagagcactgagctccacccttttctgagagttattacagccagaaagtgtgggctgaagatggttggtttcatgtttttgtattatgtatctttttgtatggtaaagactatattttgtacttaaccagatatatttttaccccagatggggatattctttgtaaaaaatgaaaataaagtttttttaatggaaaaaaaaa

(19) United States
(12) Patent Application Publication
(10) Pub. No.: US 2025/0354220 A1
(43) Pub. Date: Nov. 20, 2025

(54) METHODS FOR PROSTATE CANCER DETECTION IN SALIVA

(71) Applicant: Liquid Biopsy Research LLC, Charlestown (KN)

(72) Inventors: Irvin Mark Modlin, Woodbridge, CT (US); Mark KIDD, New Haven, CT (US); Ignat DROZDOV, Stratford Upon Avon (GB)

(21) Appl. No.: 19/120,295

(22) PCT Filed: Oct. 12, 2023
(86) PCT No.: PCT/US2023/076706

§ 371 Date: Apr. 11, 2025

Related U.S. Application Data
(60) Provisional application No. 63/379,190, filed on Oct. 12, 2022.

(51) Int. Cl. C12Q 1/6886

(52) U.S. Cl. CPC C12Q 1/6886; C12Q 2600/158

(57) ABSTRACT

The present disclosure is directed to methods for detecting a prostrate cancer, methods for determining whether a prostrate cancer is stable or progressive, low or high Gleason grade, methods for determining the completeness of surgery, and methods for evaluating the response to a prostrate cancer therapy.

### RELATED APPLICATIONS

This application claims priority to, and the benefit of, U.S. Provisional Application No. 63/379,190, filed Oct. 12, 2022, the contents of which are incorporated herein by reference in their entireties.

### REFERENCE TO ELECTRONIC SEQUENCE LISTING

The Sequence Listing XML associated with this application is provided electronically in XML file format and is hereby incorporated by reference into the specification. The name of the XML file containing the Sequence Listing XML is “LBIO-008_001WO_SeqList.xml”. The XML file is 125,439 bytes and was created on Oct. 11, 2023.

### BACKGROUND

Prostate cancer (PCa) is the fourth most commonly diagnosed cancer worldwide and the second most common cancer in men. Although the incidence and prevalence have been decreasing, ˜200,000 men will be diagnosed in the USA with PCa annually. Multiple factors including age and family history, genetic susceptibility and ethnicity all contribute to the high incidence of the disease. While 90% of PCa are diagnosed while they are localized (non-disseminated), the clinical behavior of tumors is highly variable and ranges from indolence that can be monitored through watchful waiting or active surveillance (e.g., biomarkers and 6 monthly digital rectal examination) to malignant evolution and androgen-resistant disease, metastatic dissemination and death. Symptoms of prostate cancers include problems urinating, blood in the urine or semen, trouble getting an erection, pain in the hips, back (spine), chest (ribs), or other areas from cancer that has spread to bones, weakness or numbness in the legs or feet, or even loss of bladder or bowel control from cancer pressing on the spinal cord.

Multiple risk stratification systems have been developed that combines clinical data and pathological information e.g., Gleason score. These, including the more recently developed next generation tools, are only ˜70% accurate for predicting outcome.

Molecular genetic information is increasingly being used to inform pathology and better subtype cancers. This information has been used as both prognostic tools as well as to stratify patients for different therapeutic interventions. Prostate cancers have been examined and mutations, DNA copy number alterations, rearrangements and gene fusions have all been identified. These may correlate with some pathological features. For example, low-Gleason tumors have few DNA copy number alterations while high grade tumors exhibit significant genome-wide copy number alterations. Somatic point mutations in contrast are relatively uncommon with the frequency of mutations ranging from 1% (IDH1) to 11% (SPOP). The most common abnormality is androgen-regulated fusions of ERG and other ETS family members (˜50% of tumors). However, fusion-bearing tumors do not have a significantly different prognosis to fusion-negative tumors after prostatectomy. Androgen receptor variant 7 (AR-V7) in contrast is implicated in the progression to castration resistance prostate cancer (CRPC) and is considered potentially useful as a treatment selection biomarker. Overall, however, there is an incomplete understanding of the molecular mechanisms underpinning PCa pathogenesis and an absence of molecular-based biomarkers that can be used to predict sensitivity to therapeutic agents. Consequently, the development of diagnostic methods that more accurately define disease status, identify sensitivity to therapy and can ultimately be used to better monitor disease progression, is critical.

Surveillance remains a cornerstone approach to monitor PCa and detect recurrence at an early stage. After potentially curative resection, monitoring can be undertaken through measurement of blood biomarkers and/or imaging like CT to detect asymptomatic metastatic disease earlier. The current biomarker used for monitoring is prostate specific antigen (PSA) (also gamma-seminoprotein or kallikrein-3). This glycoprotein enzyme is encoded by the KLK3 gene and is secreted by epithelial cells in the prostate. It, however, is not a unique indicator of prostate cancer, but may also detect prostatitis or benign prostatic hyperplasia (BPH). Use of PSA in isolation results in either unnecessary biopsies for men without cancer or an under diagnosis of men with significant disease. This is based on the low sensitivity (20-40%) and specificity (70-90%) ranges with a consequent positive predictive value of only 25-40%. The United States Preventive Services Task Force (USPSTF) does not recommend PSA use for prostate cancer. PSA, however, is included in clinical nomograms e.g., the UCSF-CAPRA score for prostate cancer risk, which has some utility in predicting disease free survival after surgery.

Saliva is an important testing compartment that allows evaluation of biomarkers for viral, bacterial, and fungal parasitic infections as well as for the measurement of markers that characterize systemic and non-systemic disease. Human RNA obtained from cell-free saliva has been evaluated using sequencing and PCR technologies. Cell-free RNA from healthy individuals contains more than 3,000 species of mRNA. RNA typically enters the oral cavity through secretion (from the parotid, submandibular and sublingual glands) as a component of gingival crevice fluid and from desquamated oral epithelial cells. RNA can originate form acinar cells or by circulation.

Saliva has been determined as a testing compartment for other cancers e.g., head and neck tumors. Typically, viral DNA (HPV) is isolated and amplified. This is used to provide a diagnosis of the disease. Recently, tumor RNA has been detected in saliva. For example, a 4 gene RNA based biomarker was developed for the diagnosis of oral cancer. The source of RNA may be from the salivary glands themselves or be secondary to cells e.g., lymphocytes, that are secreted into the mouth. It is also known that salivary glands are vascularized and filter blood products. This suggests that blood may also be a source of RNA detectable in saliva.

### PCa

### SUMMARY

The present disclosure provides methods of identifying the presence or absence of prostate cancer in a subject in need thereof, the methods comprising: (a) determining the expression level of at least 24 biomarkers in a test sample from the subject, wherein the at least 24 biomarkers comprise AAMP, CHTOP, EDC4, FYCO1, HNRNPU, HPN, KRT23, MAN2B2, MAX, MRPS25, NDUFS2, PPRC1, RAD23A, REPIN1, SDR39U1, SETBP1, SLC18A2, SMC4, SPARC, SQLE, STRIP1, STX12, UNC45A, XPC, and a housekeeping gene; (b) normalizing the expression level of each of AAMP, CHTOP, EDC4, FYCO1, HNRNPU, HPN, KRT23, MAN2B2, MAX, MRPS25, NDUFS2, PPRC1, RAD23A, REPIN1, SDR39U1, SETBP1, SLC18A2, SMC4, SPARC, SQLE, STRIP1, STX12, UNC45A, and XPC to the expression level of the housekeeping gene, thereby obtaining a normalized expression level of each of AAMP, CHTOP, EDC4, FYCO1, HNRNPU, HPN, KRT23, MAN2B2, MAX, MRPS25, NDUFS2, PPRC1, RAD23A, REPIN1, SDR39U1, SETBP1, SLC18A2, SMC4, SPARC, SQLE, STRIP1, STX12, UNC45A, and XPC; (c) inputting each normalized expression from step (b) into an algorithm to generate a score; (d) comparing the score to a predetermined cutoff value; and (e) identifying the presence of prostate cancer in the subject when the score is greater than or equal to the predetermined cutoff value or determining the absence of prostate cancer in the subject when the score is less than the predetermined cutoff value. In some aspects, a predetermined cutoff value is 23% on a scale of 0-100%.

The present disclosure provides methods of determining whether a prostate cancer in a subject is stable or progressive, the methods comprising: (a) determining the expression level of at least 24 biomarkers in a test sample from the subject, wherein the at least 24 biomarkers comprise AAMP, CHTOP, EDC4, FYCO1, HNRNPU, HPN, KRT23, MAN2B2, MAX, MRPS25, NDUFS2, PPRC1, RAD23A, REPIN1, SDR39U1, SETBP1, SLC18A2, SMC4, SPARC, SQLE, STRIP1, STX12, UNC45A, XPC, and a housekeeping gene; (b) normalizing the expression level of each of AAMP, CHTOP, EDC4, FYCO1, HNRNPU, HPN, KRT23, MAN2B2, MAX, MRPS25, NDUFS2, PPRC1, RAD23A, REPIN1, SDR39U1, SETBP1, SLC18A2, SMC4, SPARC, SQLE, STRIP1, STX12, UNC45A, and XPC to the expression level of the housekeeping gene, thereby obtaining a normalized expression level of each of AAMP, CHTOP, EDC4, FYCO1, HNRNPU, HPN, KRT23, MAN2B2, MAX, MRPS25, NDUFS2, PPRC1, RAD23A, REPIN1, SDR39U1, SETBP1, SLC18A2, SMC4, SPARC, SQLE, STRIP1, STX12, UNC45A, and XPC; (c) inputting each normalized expression level from step (b) into an algorithm to generate a score; (d) comparing the score to a predetermined cutoff value; and (e) determining that the prostate cancer is progressive when the score is greater than or equal to the predetermined cutoff value or determining that the prostate cancer is stable when the score is less than the predetermined cutoff value. In some aspects, the predetermined cutoff value is 50% on a scale of 0-100%.

The present disclosure provides methods of determining whether a prostate cancer in a subject has a low Gleason score (≤6) or a high Gleason score (≥7), the methods comprising: (a) determining the expression level of at least 24 biomarkers in a test sample from the subject, wherein the at least 24 biomarkers comprise AAMP, CHTOP, EDC4, FYCO1, HNRNPU, HPN, KRT23, MAN2B2, MAX, MRPS25, NDUFS2, PPRC1, RAD23A, REPIN1, SDR39U1, SETBP1, SLC18A2, SMC4, SPARC, SQLE, STRIP1, STX12, UNC45A, and XPC, and a housekeeping gene; (b) normalizing the expression level of each of AAMP, CHTOP, EDC4, FYCO1, HNRNPU, HPN, KRT23, MAN2B2, MAX, MRPS25, NDUFS2, PPRC1, RAD23A, REPIN1, SDR39U1, SETBP1, SLC18A2, SMC4, SPARC, SQLE, STRIP1, STX12, UNC45A, and XPC to the expression level of the housekeeping gene, thereby obtaining a normalized expression level of each of AAMP, CHTOP, EDC4, FYCO1, HNRNPU, HPN, KRT23, MAN2B2, MAX, MRPS25, NDUFS2, PPRC1, RAD23A, REPIN1, SDR39U1, SETBP1, SLC18A2, SMC4, SPARC, SQLE, STRIP1, STX12, UNC45A, and XPC; (c) inputting each normalized expression level from step (b) into an algorithm to generate a score; (d) comparing the score to a predetermined cutoff value; and (e) determining that the prostate cancer in the subject has a high Gleason score (≥7) when the score is greater than or equal to the predetermined cutoff value or determining that the prostate cancer in the subject has a low Gleason score (≤6) when the score is less than the predetermined cutoff value. In some aspects, the predetermined cutoff value is 50% on a scale of 0-100%.

The present disclosure provides methods of determining the completeness of surgery in a subject having a prostate cancer, the method comprising: (a) determining the expression level of at least 24 biomarkers in a test sample from the subject after the surgery, wherein the 24 biomarkers comprise AAMP, CHTOP, EDC4, FYCO1, HNRNPU, HPN, KRT23, MAN2B2, MAX, MRPS25, NDUFS2, PPRC1, RAD23A, REPIN1, SDR39U1, SETBP1, SLC18A2, SMC4, SPARC, SQLE, STRIP1, STX12, UNC45A, XPC, and a housekeeping gene; (b) normalizing the expression level of each of AAMP, CHTOP, EDC4, FYCO1, HNRNPU, HPN, KRT23, MAN2B2, MAX, MRPS25, NDUFS2, PPRC1, RAD23A, REPIN1, SDR39U1, SETBP1, SLC18A2, SMC4, SPARC, SQLE, STRIP1, STX12, UNC45A, and XPC to the expression level of the housekeeping gene, thereby obtaining a normalized expression level of each of AAMP, CHTOP, EDC4, FYCO1, HNRNPU, HPN, KRT23, MAN2B2, MAX, MRPS25, NDUFS2, PPRC1, RAD23A, REPIN1, SDR39U1, SETBP1, SLC18A2, SMC4, SPARC, SQLE, STRIP1, STX12, UNC45A, and XPC; (c) inputting each normalized expression level from step (b) into an algorithm to generate a score; (d) comparing the score to a predetermined cutoff value; and (e) identifying that the prostate cancer is not completely removed when the score is greater than or equal to the score or identifying that the prostate cancer is completely removed when the score is less than the predetermined cutoff value. In some aspects, the predetermined cutoff value is 50% on a scale of 0-100%.

The present disclosure provide methods of evaluating the response of a subject having a prostate cancer to an anti-prostate cancer therapy, the methods comprising: (a) at a first time point: (i) determining the expression level of at least 24 biomarkers in a test sample from the subject, wherein the at least 24 biomarkers comprise AAMP, CHTOP, EDC4, FYCO1, HNRNPU, HPN, KRT23, MAN2B2, MAX, MRPS25, NDUFS2, PPRC1, RAD23A, REPIN1, SDR39U1, SETBP1, SLC18A2, SMC4, SPARC, SQLE, STRIP1, STX12, UNC45A, XPC, and a housekeeping gene; (ii) normalizing the expression level of each of AAMP, CHTOP, EDC4, FYCO1, HNRNPU, HPN, KRT23, MAN2B2, MAX, MRPS25, NDUFS2, PPRC1, RAD23A, REPIN1, SDR39U1, SETBP1, SLC18A2, SMC4, SPARC, SQLE, STRIP1, STX12, UNC45A, and XPC to the expression level of the housekeeping gene, thereby obtaining a normalized expression level of each of AAMP, CHTOP, EDC4, FYCO1, HNRNPU, HPN, KRT23, MAN2B2, MAX, MRPS25, NDUFS2, PPRC1, RAD23A, REPIN1, SDR39U1, SETBP1, SLC18A2, SMC4, SPARC, SQLE, STRIP1, STX12, UNC45A, and XPC; and (iii) inputting each normalized expression level from step (a)(ii) into an algorithm to generate a first score; (b) at a second time point, wherein the second time point is after the first time point and after the administration of the therapy to the subject: (i) determining the expression level of the at least 24 biomarkers in a test sample from the subject; (ii) normalizing the expression level of each of AAMP, CHTOP, EDC4, FYCO1, HNRNPU, HPN, KRT23, MAN2B2, MAX, MRPS25, NDUFS2, PPRC1, RAD23A, REPIN1, SDR39U1, SETBP1, SLC18A2, SMC4, SPARC, SQLE, STRIP1, STX12, UNC45A, and XPC to the expression level of the housekeeping gene, thereby obtaining a normalized expression level of each of AAMP, CHTOP, EDC4, FYCO1, HNRNPU, HPN, KRT23, MAN2B2, MAX, MRPS25, NDUFS2, PPRC1, RAD23A, REPIN1, SDR39U1, SETBP1, SLC18A2, SMC4, SPARC, SQLE, STRIP1, STX12, UNC45A, and XPC; and (iii) inputting each normalized expression level from step (b)(ii) into an algorithm to generate a second score; (c) comparing the first score and second score; and (d) identifying that the subject is responsive to the anti-prostate cancer therapy when the second score is decreased as compared to the first score or identifying that the subject is not responsive to the anti-prostate cancer therapy when the second score is not decreased as compared to the normalized expression levels from step (a)(ii). In some aspects, the subject is identified as responsive to the anti-neuroendocrine cancer therapy when the second is at least 5% less than the first score.

In some aspects of the preceding methods, the housekeeping gene is selected from the group consisting of ATG4B, RHOA, TOX4, TPT1, and TXNIP. In some aspects, the housekeeping gene is TOX4.

In some aspects, the preceding methods have a sensitivity of at least 90%.

In some aspects, the preceding methods have a specificity of at least 90%.

In some aspects of the preceding methods, at least one of the at least 24 biomarkers is RNA, cDNA, or protein. In some aspects, wherein when the biomarker is RNA, the RNA is reverse transcribed to produce cDNA, and the produced cDNA expression level is detected.

In some aspects of the preceding methods, the expression level of the biomarker is detected by forming a complex between the biomarker and a labeled probe or primer. In some aspects, the label is a fluorescent label.

In some aspects, wherein when the biomarker is protein, the protein detected by forming a complex between the protein and a labeled antibody.

In some aspects, wherein when the biomarker is RNA or cDNA, the RNA or cDNA is detected by forming a complex between the RNA or cDNA and a labeled nucleic acid probe or primer. In some aspects, the complex between the RNA or cDNA and the labeled nucleic acid probe or primer is a hybridization complex.

In some aspects of the preceding methods, the first predetermined cutoff value is derived from a plurality of reference samples obtained from subjects not having or not diagnosed with a neoplastic disease. In some aspects, the neoplastic disease is prostate cancer.

In some aspects of the preceding methods, the algorithm is XGB, RF, glmnet, cforest, CART, treebag, knn, nnet, SVM-radial, SVM-linear, NB, or mlp. In some aspects of the preceding methods, the algorithm is Random Forest.

In some aspects of the preceding methods, the machine learning algorithm is trained using the expression levels or normalized expression levels of the at least 24 biomarkers obtained from a plurality of reference samples obtained from subjects not having a neuroendocrine cancer and the expression levels or normalized expression levels of the at least 24 biomarkers from a plurality of reference samples obtained from subjects having neuroendocrine cancer.

In some aspects, the preceding methods further comprise treat the subject identified as having prostate cancer with at least one anti-prostate cancer therapy.

In some aspects, the anti-prostate cancer therapy comprises comprise active surveillance, surgery, radiation therapy, cryotherapy, hormone therapy, chemotherapy, vaccine treatment, bone-directed treatment, or any combination thereof.

In some aspects, a radiation therapy comprises external beam radiation, brachytherapy, radiopharmaceuticals or any combination thereof, preferably wherein the radiopharmaceuticals comprise ¹⁷⁷Lu-PSMA.

In some aspects, a hormone therapy comprises androgen suppression therapy.

In some aspects, a chemotherapy comprises docetaxel, cabazitaxel, mitoxantrone, estramustine, or any combination thereof.

In some aspects, a vaccine treatment comprises Sipuleucel-T.

In some aspects, a bone-directed treatment comprises a bisphosphonate, denosumab, a corticosteroid, or a combination thereof.

In some aspects of the preceding methods, the first time point is prior to the administration of the therapy to the subject.

In some aspects of the preceding methods, the first time point is after the administration of the therapy to the subject.

In some aspects, the test sample is saliva.

In some aspects, the test sample is self-collected saliva into a container with stabilization fluid.

### BRIEF DESCRIPTION OF THE DRAWINGS

FIG. 1 is a graph showing the relationship between gene expression in blood and in saliva.

FIG. 2A and FIG. 2B are X-Y scatter graphs showing the concordance between Ct values in blood and saliva (FIG. 2A) and normalized gene expression in blood and saliva (FIG. 2B). Red line is the linear correlation. Vertical and horizontal lines SEM and SD, respectively of the averaged values from the 36 target genes.

FIG. 3 is a graph showing the relationship between normalized gene expression in tumour samples and in saliva. Red line is the linear correlation. Vertical and horizontal lines SEM and SD, respectively of the averaged values from the 24 target genes.

FIG. 4 is a graph showing gene expression in age/sex-matched controls: n=30, and neuroendocrine cancer cases: n=15). Expression levels were significantly (p<0.05) elevated in 14 genes and was significantly decreased in 5 of the target genes.

FIG. 5A and FIG. 5B are graphs showing visualisation of 24 putative marker genes identified by the Random Forest algorithm in the derivation cohort of n=163 control samples and 51 cancer samples. (FIG. 5A) Expression normalized to TOX4. (FIG. 5B) Expression normalized to TPT1.

FIG. 6 is a graph showing SalivaPROSTest scores in an independent set of controls (n=100) and neuroendocrine cancers (n=40). Levels were significantly elevated (p<0.0001) in NETs (61±24) versus controls (6±5).

FIG. 7 is a graph showing receiver operator curve analysis of the testing partition in the independent set. The AUROC was 0.99. The Youden J index was 0.95. The Z-statistic was highly significant (304.7; p<0.0001).

FIG. 8 is a graph showing the metrics of the assay for determining prostate cancers. The sensitivity was 95% and specificity was 100%.

FIG. 9 is a graph showing SalivaPROSTest scores in high grade PCas (Gleason≥7) compared to low grade (Gleason 5+6) tumors. Levels were significantly elevated (p<0.002) in higher grade tumors (72±25) versus low Gleason tumors (46±19).

FIG. 10 is a graph showing the effect of surgery on the SalivaPROSTest. Levels prior to surgery are elevated (64±18%). Surgery reduced levels to 33±11% (p<0.0001), not different to control levels.

FIG. 11A and FIG. 11B are spider plot graphs showing the effect of treatment on the SalivaPROSTest test. Levels prior to treatment are elevated (69±23%). In those who responded to therapy, levels were reduced by −38±31% and −60±19% at the two follow-up time points (p 0.0001). In those who progressed despite therapy, levels were increased by ±18±19% and ±27±7% (p<0.05), respectively. (FIG. 11A) Follow-up plot of all patients. (FIG. 11B) Spider plot of individual responders (blue) and those who progress (red).

### DETAILED DESCRIPTION

The details of the inventions are set forth in the accompanying description below. Although methods and materials similar or equivalent to those described herein can be used in the practice or testing of the present inventions, illustrative methods and materials are now described. Other features, objects, and advantages of the inventions will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which these inventions belongs. All patents and publications cited in this specification are incorporated herein by reference in their entireties.

Described herein are methods to quantitate (score) a salivary prostate cancer molecular signature with high sensitivity and specificity for purposes including, but not limited to, detecting a prostate cancer, determining whether a prostate cancer is stable or progressive, determining the completeness of surgery, and evaluating the response of a subject to a prostate cancer therapy, treating prostate cancer in a subject, or any combination thereof. Without wishing to be bound by theory, the present inventions are based on the discovery that the expression levels of AAMP, CHTOP, EDC4, FYCO1, HNRNPU, HPN, KRT23, MAN2B2, MAX, MRPS25, NDUFS2, PPRC1, RAD23A, REPIN1, SDR39U1, SETBP1, SLC18A2, SMC4, SPARC, SQLE, STRIP1, STX12, UNC45A, and XPC, normalized by the expression level of a housekeeping gene, are elevated in subjects having prostate cancers as compared to healthy subjects.

As described herein, measurements of the expression level of the circulating prostate cancer transcripts described above (referred to collectively as the “SalivaPROSTest transcripts”) in a saliva sample from a subject can be used to diagnoses prostate cancer. In non-limiting examples, the expression levels of the SalivaPROSTest transcripts as measured from a saliva sample can be inputted into an algorithm to generate a score (referred to herein as the “ProstaTest score”), which can be used to diagnose the presence of prostate cancer in a subject. Moreover, decreases in a subject's ProstaTest score after administration of one or more anti-prostate cancer therapies (e.g. surgery and chemotherapy) can be used to determine the subject's responsiveness to the one or more therapies, optionally in combination with standard clinical assessment and imaging.

The present disclosure provides methods of identifying the presence or absence of prostate cancer in a subject in need thereof, the method comprising: (a) determining the expression level of at least 24 biomarkers in a test sample from the subject, wherein the at least 24 biomarkers comprise AAMP, CHTOP, EDC4, FYCO1, HNRNPU, HPN, KRT23, MAN2B2, MAX, MRPS25, NDUFS2, PPRC1, RAD23A, REPIN1, SDR39U1, SETBP1, SLC18A2, SMC4, SPARC, SQLE, STRIP1, STX12, UNC45A, XPC, and a housekeeping gene; (b) normalizing the expression level of each of AAMP, CHTOP, EDC4, FYCO1, HNRNPU, HPN, KRT23, MAN2B2, MAX, MRPS25, NDUFS2, PPRC1, RAD23A, REPIN1, SDR39U1, SETBP1, SLC18A2, SMC4, SPARC, SQLE, STRIP1, STX12, UNC45A, and XPC to the expression level of the housekeeping gene, thereby obtaining a normalized expression level of each of AAMP, CHTOP, EDC4, FYCO1, HNRNPU, HPN, KRT23, MAN2B2, MAX, MRPS25, NDUFS2, PPRC1, RAD23A, REPIN1, SDR39U1, SETBP1, SLC18A2, SMC4, SPARC, SQLE, STRIP1, STX12, UNC45A, and XPC; and (c) identifying the presence or absence of prostate cancer in the subject based on the normalized expression levels from step (b). In some aspects, identifying the presence of absence of prostate cancer in the subject based on the normalized expression levels from step (b) can comprise comparing the normalized expression levels to corresponding predetermined cutoff values and identifying the presence or absence of the prostate cancer in the subject based on the relationship between the normalized expression levels and the corresponding predetermined cutoff values (e.g. greater than, greater than or equal to, less than, less than or equal to, or equal to).

The present disclosure provides methods of identifying the presence or absence of prostate cancer in a subject in need thereof, the method comprising: (a) determining the expression level of at least 24 biomarkers in a test sample from the subject, wherein the at least 24 biomarkers comprise AAMP, CHTOP, EDC4, FYCO1, HNRNPU, HPN, KRT23, MAN2B2, MAX, MRPS25, NDUFS2, PPRC1, RAD23A, REPIN1, SDR39U1, SETBP1, SLC18A2, SMC4, SPARC, SQLE, STRIP1, STX12, UNC45A, XPC, and a housekeeping gene; (b) normalizing the expression level of each of AAMP, CHTOP, EDC4, FYCO1, HNRNPU, HPN, KRT23, MAN2B2, MAX, MRPS25, NDUFS2, PPRC1, RAD23A, REPIN1, SDR39U1, SETBP1, SLC18A2, SMC4, SPARC, SQLE, STRIP1, STX12, UNC45A, and XPC to the expression level of the housekeeping gene, thereby obtaining a normalized expression level of each of AAMP, CHTOP, EDC4, FYCO1, HNRNPU, HPN, KRT23, MAN2B2, MAX, MRPS25, NDUFS2, PPRC1, RAD23A, REPIN1, SDR39U1, SETBP1, SLC18A2, SMC4, SPARC, SQLE, STRIP1, STX12, UNC45A, and XPC; (c) inputting each normalized expression from step (b) into an algorithm to generate a score; and (d) identifying the presence or absence of prostate cancer in the subject based on the score. In some aspects, identifying the presence of absence of prostate cancer in the subject based on the score can comprise comparing the score to a predetermined cutoff value and identifying the presence or absence of the prostate cancer in the subject based on the relationship between the score and the predetermined cutoff value (e.g. greater than, greater than or equal to, less than, less than or equal to, or equal to).

The present disclosure provides methods of identifying the presence or absence of prostate cancer in a subject in need thereof, the method comprising: (a) determining the expression level of at least 24 biomarkers in a test sample from the subject, wherein the at least 24 biomarkers comprise AAMP, CHTOP, EDC4, FYCO1, HNRNPU, HPN, KRT23, MAN2B2, MAX, MRPS25, NDUFS2, PPRC1, RAD23A, REPIN1, SDR39U1, SETBP1, SLC18A2, SMC4, SPARC, SQLE, STRIP1, STX12, UNC45A, XPC, and a housekeeping gene; (b) normalizing the expression level of each of AAMP, CHTOP, EDC4, FYCO1, HNRNPU, HPN, KRT23, MAN2B2, MAX, MRPS25, NDUFS2, PPRC1, RAD23A, REPIN1, SDR39U1, SETBP1, SLC18A2, SMC4, SPARC, SQLE, STRIP1, STX12, UNC45A, and XPC to the expression level of the housekeeping gene, thereby obtaining a normalized expression level of each of AAMP, CHTOP, EDC4, FYCO1, HNRNPU, HPN, KRT23, MAN2B2, MAX, MRPS25, NDUFS2, PPRC1, RAD23A, REPIN1, SDR39U1, SETBP1, SLC18A2, SMC4, SPARC, SQLE, STRIP1, STX12, UNC45A, and XPC; (c) inputting each normalized expression from step (b) into an algorithm to generate a score; (d) comparing the score to a predetermined cutoff value; and (e) identifying the presence of prostate cancer in the subject when the score is greater than or equal to the predetermined cutoff value or determining the absence of prostate cancer in the subject when the score is less than the predetermined cutoff value.

The present disclosure provides methods of identifying the presence or absence of prostate cancer in a subject in need thereof, the method comprising: (a) determining the expression level of at least 24 biomarkers in a test sample from the subject, wherein the at least 24 biomarkers comprise AAMP, CHTOP, EDC4, FYCO1, HNRNPU, HPN, KRT23, MAN2B2, MAX, MRPS25, NDUFS2, PPRC1, RAD23A, REPIN1, SDR39U1, SETBP1, SLC18A2, SMC4, SPARC, SQLE, STRIP1, STX12, UNC45A, XPC, and a housekeeping gene; (b) normalizing the expression level of each of AAMP, CHTOP, EDC4, FYCO1, HNRNPU, HPN, KRT23, MAN2B2, MAX, MRPS25, NDUFS2, PPRC1, RAD23A, REPIN1, SDR39U1, SETBP1, SLC18A2, SMC4, SPARC, SQLE, STRIP1, STX12, UNC45A, and XPC to the expression level of the housekeeping gene, thereby obtaining a normalized expression level of each of AAMP, CHTOP, EDC4, FYCO1, HNRNPU, HPN, KRT23, MAN2B2, MAX, MRPS25, NDUFS2, PPRC1, RAD23A, REPIN1, SDR39U1, SETBP1, SLC18A2, SMC4, SPARC, SQLE, STRIP1, STX12, UNC45A, and XPC; (c) inputting each normalized expression from step (b) into an algorithm to generate a score; (d) comparing the score to a predetermined cutoff value; and (e) identifying the presence of prostate cancer in the subject when the score is greater than the predetermined cutoff value or determining the absence of prostate cancer in the subject when the score is less than or equal to the predetermined cutoff value.

In some aspects of the preceding methods, the predetermined cutoff value can be 23% on a scale of 0-100%.

The present disclosure provides methods of identifying the risk of a subject having prostate cancer, the method comprising: (a) determining the expression level of at least 24 biomarkers in a test sample from the subject, wherein the at least 24 biomarkers comprise AAMP, CHTOP, EDC4, FYCO1, HNRNPU, HPN, KRT23, MAN2B2, MAX, MRPS25, NDUFS2, PPRC1, RAD23A, REPIN1, SDR39U1, SETBP1, SLC18A2, SMC4, SPARC, SQLE, STRIP1, STX12, UNC45A, XPC, and a housekeeping gene; (b) normalizing the expression level of each of AAMP, CHTOP, EDC4, FYCO1, HNRNPU, HPN, KRT23, MAN2B2, MAX, MRPS25, NDUFS2, PPRC1, RAD23A, REPIN1, SDR39U1, SETBP1, SLC18A2, SMC4, SPARC, SQLE, STRIP1, STX12, UNC45A, and XPC to the expression level of the housekeeping gene, thereby obtaining a normalized expression level of each of AAMP, CHTOP, EDC4, FYCO1, HNRNPU, HPN, KRT23, MAN2B2, MAX, MRPS25, NDUFS2, PPRC1, RAD23A, REPIN1, SDR39U1, SETBP1, SLC18A2, SMC4, SPARC, SQLE, STRIP1, STX12, UNC45A, and XPC; and (c) identifying the risk of the subject having prostate cancer based on the normalized expression levels from step (b). In some aspects, identifying the risk of the subject having prostate cancer based on the normalized expression levels from step (b) can comprise comparing the normalized expression levels to corresponding predetermined cutoff values and identifying the risk of the subject having prostate cancer based on the relationship between the normalized expression levels and the corresponding predetermined cutoff values (e.g. greater than, greater than or equal to, less than, less than or equal to, or equal to).

The present disclosure provides methods of identifying the risk of a subject having prostate cancer, the method comprising: (a) determining the expression level of at least 24 biomarkers in a test sample from the subject, wherein the at least 24 biomarkers comprise AAMP, CHTOP, EDC4, FYCO1, HNRNPU, HPN, KRT23, MAN2B2, MAX, MRPS25, NDUFS2, PPRC1, RAD23A, REPIN1, SDR39U1, SETBP1, SLC18A2, SMC4, SPARC, SQLE, STRIP1, STX12, UNC45A, XPC, and a housekeeping gene; (b) normalizing the expression level of each of AAMP, CHTOP, EDC4, FYCO1, HNRNPU, HPN, KRT23, MAN2B2, MAX, MRPS25, NDUFS2, PPRC1, RAD23A, REPIN1, SDR39U1, SETBP1, SLC18A2, SMC4, SPARC, SQLE, STRIP1, STX12, UNC45A, and XPC to the expression level of the housekeeping gene, thereby obtaining a normalized expression level of each of AAMP, CHTOP, EDC4, FYCO1, HNRNPU, HPN, KRT23, MAN2B2, MAX, MRPS25, NDUFS2, PPRC1, RAD23A, REPIN1, SDR39U1, SETBP1, SLC18A2, SMC4, SPARC, SQLE, STRIP1, STX12, UNC45A, and XPC; (c) inputting each normalized expression from step (b) into an algorithm to generate a score; and (d) identifying the risk of the subject having prostate based on the score. In some aspects, identifying the risk of the subject having prostate cancer based on the score can comprise comparing the score to a predetermined cutoff value and the risk of the subject having prostate based on the relationship between the score and the predetermined cutoff value (e.g. greater than, greater than or equal to, less than, less than or equal to, or equal to).

Accordingly, the present disclosure provides methods of identifying the risk of a subject having prostate cancer, the method comprising: (a) determining the expression level of at least 24 biomarkers in a test sample from the subject, wherein the at least 24 biomarkers comprise AAMP, CHTOP, EDC4, FYCO1, HNRNPU, HPN, KRT23, MAN2B2, MAX, MRPS25, NDUFS2, PPRC1, RAD23A, REPIN1, SDR39U1, SETBP1, SLC18A2, SMC4, SPARC, SQLE, STRIP1, STX12, UNC45A, XPC, and a housekeeping gene; (b) normalizing the expression level of each of AAMP, CHTOP, EDC4, FYCO1, HNRNPU, HPN, KRT23, MAN2B2, MAX, MRPS25, NDUFS2, PPRC1, RAD23A, REPIN1, SDR39U1, SETBP1, SLC18A2, SMC4, SPARC, SQLE, STRIP1, STX12, UNC45A, and XPC to the expression level of the housekeeping gene, thereby obtaining a normalized expression level of each of AAMP, CHTOP, EDC4, FYCO1, HNRNPU, HPN, KRT23, MAN2B2, MAX, MRPS25, NDUFS2, PPRC1, RAD23A, REPIN1, SDR39U1, SETBP1, SLC18A2, SMC4, SPARC, SQLE, STRIP1, STX12, UNC45A, and XPC; (c) inputting each normalized expression from step (b) into an algorithm to generate a score; (d) comparing the score to a predetermined cutoff value; and (e) identifying that the subject is at high risk of having prostate cancer when the score is greater than or equal to the predetermined cutoff value or determining that the subject is at a low risk of having prostate cancer when the score is less than the predetermined cutoff value.

Accordingly, the present disclosure provides methods of identifying the risk of a subject having prostate cancer, the method comprising: (a) determining the expression level of at least 24 biomarkers in a test sample from the subject, wherein the at least 24 biomarkers comprise AAMP, CHTOP, EDC4, FYCO1, HNRNPU, HPN, KRT23, MAN2B2, MAX, MRPS25, NDUFS2, PPRC1, RAD23A, REPIN1, SDR39U1, SETBP1, SLC18A2, SMC4, SPARC, SQLE, STRIP1, STX12, UNC45A, XPC, and a housekeeping gene; (b) normalizing the expression level of each of AAMP, CHTOP, EDC4, FYCO1, HNRNPU, HPN, KRT23, MAN2B2, MAX, MRPS25, NDUFS2, PPRC1, RAD23A, REPIN1, SDR39U1, SETBP1, SLC18A2, SMC4, SPARC, SQLE, STRIP1, STX12, UNC45A, and XPC to the expression level of the housekeeping gene, thereby obtaining a normalized expression level of each of AAMP, CHTOP, EDC4, FYCO1, HNRNPU, HPN, KRT23, MAN2B2, MAX, MRPS25, NDUFS2, PPRC1, RAD23A, REPIN1, SDR39U1, SETBP1, SLC18A2, SMC4, SPARC, SQLE, STRIP1, STX12, UNC45A, and XPC; (c) inputting each normalized expression from step (b) into an algorithm to generate a score; (d) comparing the score to a predetermined cutoff value; and (e) identifying that the subject is at high risk of having prostate cancer when the score is greater than the predetermined cutoff value or determining that the subject is at a low risk of having prostate cancer when the score is less than or equal to the predetermined cutoff value.

In some aspects of the preceding methods, the predetermined cutoff value can be 23% on a scale of 0-100%.

The present disclosure also provides methods of determining whether a prostate cancer in a subject is stable or progressive, the method comprising: (a) determining the expression level of at least 24 biomarkers in a test sample from the subject, wherein the at least 24 biomarkers comprise AAMP, CHTOP, EDC4, FYCO1, HNRNPU, HPN, KRT23, MAN2B2, MAX, MRPS25, NDUFS2, PPRC1, RAD23A, REPIN1, SDR39U1, SETBP1, SLC18A2, SMC4, SPARC, SQLE, STRIP1, STX12, UNC45A, XPC, and a housekeeping gene; (b) normalizing the expression level of each of AAMP, CHTOP, EDC4, FYCO1, HNRNPU, HPN, KRT23, MAN2B2, MAX, MRPS25, NDUFS2, PPRC1, RAD23A, REPIN1, SDR39U1, SETBP1, SLC18A2, SMC4, SPARC, SQLE, STRIP1, STX12, UNC45A, and XPC to the expression level of the housekeeping gene, thereby obtaining a normalized expression level of each of AAMP, CHTOP, EDC4, FYCO1, HNRNPU, HPN, KRT23, MAN2B2, MAX, MRPS25, NDUFS2, PPRC1, RAD23A, REPIN1, SDR39U1, SETBP1, SLC18A2, SMC4, SPARC, SQLE, STRIP1, STX12, UNC45A, and XPC; and (c) determining whether the prostate cancer in the subject is stable or progressive based on the normalized expression levels from step (b). In some aspects, determining whether the prostate cancer in the subject is stable or progressive based on the normalized expression levels from step (b) comprises comparing the normalized expression levels to corresponding predetermined cutoff values and determining whether the prostate cancer in the subject is stable or progressive based on the relationship between the normalized expression levels and the corresponding predetermined cutoff values (e.g. greater than, greater than or equal to, less than, less than or equal to, or equal to).

The present disclosure also provides methods of determining whether a prostate cancer in a subject is stable or progressive, the method comprising: (a) determining the expression level of at least 24 biomarkers in a test sample from the subject, wherein the at least 24 biomarkers comprise AAMP, CHTOP, EDC4, FYCO1, HNRNPU, HPN, KRT23, MAN2B2, MAX, MRPS25, NDUFS2, PPRC1, RAD23A, REPIN1, SDR39U1, SETBP1, SLC18A2, SMC4, SPARC, SQLE, STRIP1, STX12, UNC45A, XPC, and a housekeeping gene; (b) normalizing the expression level of each of AAMP, CHTOP, EDC4, FYCO1, HNRNPU, HPN, KRT23, MAN2B2, MAX, MRPS25, NDUFS2, PPRC1, RAD23A, REPIN1, SDR39U1, SETBP1, SLC18A2, SMC4, SPARC, SQLE, STRIP1, STX12, UNC45A, and XPC to the expression level of the housekeeping gene, thereby obtaining a normalized expression level of each of AAMP, CHTOP, EDC4, FYCO1, HNRNPU, HPN, KRT23, MAN2B2, MAX, MRPS25, NDUFS2, PPRC1, RAD23A, REPIN1, SDR39U1, SETBP1, SLC18A2, SMC4, SPARC, SQLE, STRIP1, STX12, UN (45A, and XPC; (c) inputting each normalized expression level from step (b) into an algorithm to generate a score; and (d) determining whether the prostate cancer in the subject is stable or progressive based on the normalized expression levels from step (b). In some aspects, determining whether the prostate cancer in the subject is stable or progressive based on the score comprises comparing the score to a predetermined cutoff value and determining whether the prostate cancer in the subject is stable or progressive based on the relationship between the score and the predetermined cutoff value (e.g. greater than, greater than or equal to, less than, less than or equal to, or equal to).

The present disclosure also provides methods of determining whether a prostate cancer in a subject is stable or progressive, the method comprising: (a) determining the expression level of at least 24 biomarkers in a test sample from the subject, wherein the at least 24 biomarkers comprise AAMP, CHTOP, EDC4, FYCO1, HNRNPU, HPN, KRT23, MAN2B2, MAX, MRPS25, NDUFS2, PPRC1, RAD23A, REPIN1, SDR39U1, SETBP1, SLC18A2, SMC4, SPARC, SQLE, STRIP1, STX12, UNC45A, XPC, and a housekeeping gene; (b) normalizing the expression level of each of AAMP, CHTOP, EDC4, FYCO1, HNRNPU, HPN, KRT23, MAN2B2, MAX, MRPS25, NDUFS2, PPRC1, RAD23A, REPIN1, SDR39U1, SETBP1, SLC18A2, SMC4, SPARC, SQLE, STRIP1, STX12, UNC45A, and XPC to the expression level of the housekeeping gene, thereby obtaining a normalized expression level of each of AAMP, CHTOP, EDC4, FYCO1, HNRNPU, HPN, KRT23, MAN2B2, MAX, MRPS25, NDUFS2, PPRC1, RAD23A, REPIN1, SDR39U1, SETBP1, SLC18A2, SMC4, SPARC, SQLE, STRIP1, STX12, UNC45A, and XPC; (c) inputting each normalized expression level from step (b) into an algorithm to generate a score; (d) comparing the score to a predetermined cutoff value; and (e) determining that the prostate cancer is progressive when the score is greater than or equal to the predetermined cutoff value or determining that the prostate cancer is stable when the score is less than the predetermined cutoff value.

The present disclosure also provides methods of determining whether a prostate cancer in a subject is stable or progressive, the method comprising: (a) determining the expression level of at least 24 biomarkers in a test sample from the subject, wherein the at least 24 biomarkers comprise AAMP, CHTOP, EDC4, FYCO1, HNRNPU, HPN, KRT23, MAN2B2, MAX, MRPS25, NDUFS2, PPRC1, RAD23A, REPIN1, SDR39U1, SETBP1, SLC18A2, SMC4, SPARC, SQLE, STRIP1, STX12, UNC45A, XPC, and a housekeeping gene; (b) normalizing the expression level of each of AAMP, CHTOP, EDC4, FYCO1, HNRNPU, HPN, KRT23, MAN2B2, MAX, MRPS25, NDUFS2, PPRC1, RAD23A, REPIN1, SDR39U1, SETBP1, SLC18A2, SMC4, SPARC, SQLE, STRIP1, STX12, UNC45A, and XPC to the expression level of the housekeeping gene, thereby obtaining a normalized expression level of each of AAMP, CHTOP, EDC4, FYCO1, HNRNPU, HPN, KRT23, MAN2B2, MAX, MRPS25, NDUFS2, PPRC1, RAD23A, REPIN1, SDR39U1, SETBP1, SLC18A2, SMC4, SPARC, SQLE, STRIP1, STX12, UNC45A, and XPC; (c) inputting each normalized expression level from step (b) into an algorithm to generate a score; (d) comparing the score to a predetermined cutoff value; and (e) determining that the prostate cancer is progressive when the score is greater than the predetermined cutoff value or determining that the prostate cancer is stable when the score is less than or equal to the predetermined cutoff value.

In some aspects of the preceding methods, the predetermined cutoff value can be 50% on a scale of 0-100%.

Additionally, the present disclosure also provides methods of determining whether a prostate cancer in a subject has a low Gleason score (≤6) or a high Gleason score (≥7), the method comprising: (a) determining the expression level of at least 24 biomarkers in a test sample from the subject, wherein the at least 24 biomarkers comprise AAMP, CHTOP, EDC4, FYCO1, HNRNPU, HPN, KRT23, MAN2B2, MAX, MRPS25, NDUFS2, PPRC1, RAD23A, REPIN1, SDR39U1, SETBP1, SLC18A2, SMC4, SPARC, SQLE, STRIP1, STX12, UNC45A, and XP (′, and a housekeeping gene; (b) normalizing the expression level of each of AAMP, CHTOP, EDC4, FYCO1, HNRNPU, HPN, KRT23, MAN2B2, MAX, MRPS25, NDUFS2, PPRC1, RAD23A, REPIN1, SDR39U1, SETBP1, SLC18A2, SMC4, SPARC, SQLE, STRIP1, STX12, UNC45A, and XPC to the expression level of the housekeeping gene, thereby obtaining a normalized expression level of each of AAMP, CHTOP, EDC4, FYCO1, HNRNPU, HPN, KRT23, MAN2B2, MAX, MRPS25, NDUFS2, PPRC1, RAD23A, REPIN1, SDR39U1, SETBP1, SLC18A2, SMC4, SPARC, SQLE, STRIP1, STX12, UNC45A, and XPC; and (c) determining whether the prostate cancer in the subject has a low Gleason score (≤6) or a high Gleason score (≥7) based on the normalized expression levels from step (b). In some aspects, determining whether the prostate cancer in the subject has a low Gleason score (≤6) or a high Gleason score (≥7) based on the normalized expression levels from step (b) comprises comparing the normalized expression levels to corresponding predetermined cutoff values and determining whether the prostate cancer in the subject has a low Gleason score (≤6) or a high Gleason score (≥7) based on the relationship between the normalized expression levels and the corresponding predetermined cutoff values (e.g. greater than, greater than or equal to, less than, less than or equal to, or equal to).

The present disclosure also provides methods of determining whether a prostate cancer in a subject has a low Gleason score (≤6) or a high Gleason score (≥7), the method comprising: (a) determining the expression level of at least 24 biomarkers in a test sample from the subject, wherein the at least 24 biomarkers comprise AAMP, CHTOP, EDC4, FYCO1, HNRNPU, HPN, KRT23, MAN2B2, MAX, MRPS25, NDUFS2, PPRC1, RAD23A, REPIN1, SDR39U1, SETBP1, SLC18A2, SMC4, SPARC, SQLE, STRIP1, STX12, UNC45A, and XPC, and a housekeeping gene; (b) normalizing the expression level of each of AAMP, CHTOP, EDC4, FYCO1, HNRNPU, HPN, KRT23, MAN2B2, MAX, MRPS25, NDUFS2, PPRC1, RAD23A, REPIN1, SDR39U1, SETBP1, SLC18A2, SMC4, SPARC, SQLE, STRIP1, STX12, UNC45A, and XPC to the expression level of the housekeeping gene, thereby obtaining a normalized expression level of each of AAMP, CHTOP, EDC4, FYCO1, HNRNPU, HPN, KRT23, MAN2B2, MAX, MRPS25, NDUFS2, PPRC1, RAD23A, REPIN1, SDR39U1, SETBP1, SLC18A2, SMC4, SPARC, SQLE, STRIP1, STX12, UNC45A, and XPC; (c) inputting each normalized expression level from step (b) into an algorithm to generate a score; and (d) determining whether the prostate cancer in the subject has a low Gleason score (≤6) or a high Gleason score (≥7) based on the score. In some aspects, determining whether the prostate cancer in the subject has a low Gleason score (≤6) or a high Gleason score (≥7) based on the score comprises comparing the score to a predetermined cutoff value and determining whether the prostate cancer in the subject has a low Gleason score (≤6) or a high Gleason score (≥7) based on the relationship between the score and the predetermined cutoff value (e.g. greater than, greater than or equal to, less than, less than or equal to, or equal to).

Accordingly, the present disclosure also provides methods of determining whether a prostate cancer in a subject has a low Gleason score (≤6) or a high Gleason score (≥7), the method comprising: (a) determining the expression level of at least 24 biomarkers in a test sample from the subject, wherein the at least 24 biomarkers comprise AAMP, CHTOP, EDC4, FYCO1, HNRNPU, HPN, KRT23, MAN2B2, MAX, MRPS25, NDUFS2, PPRC1, RAD23A, REPIN1, SDR39U1, SETBP1, SLC18A2, SMC4, SPARC, SQLE, STRIP1, STX12, UNC45A, and XPC, and a housekeeping gene; (b) normalizing the expression level of each of AAMP, CHTOP, EDC4, FYCO1, HNRNPU, HPN, KRT23, MAN2B2, MAX, MRPS25, NDUFS2, PPRC1, RAD23A, REPIN1, SDR39U1, SETBP1, SLC18A2, SMC4, SPARC, SQLE, STRIP1, STX12, UNC45A, and XPC to the expression level of the housekeeping gene, thereby obtaining a normalized expression level of each of AAMP, CHTOP, EDC4, FYCO1, HNRNPU, HPN, KRT23, MAN2B2, MAX, MRPS25, NDUFS2, PPRC1, RAD23A, REPIN1, SDR39U1, SETBP1, SLC18A2, SMC4, SPARC, SQLE, STRIP1, STX12, UNC45A, and XPC; (c) inputting each normalized expression level from step (b) into an algorithm to generate a score; (d) comparing the score to a predetermined cutoff value; and (e) determining that the prostate cancer in the subject has a high Gleason score (≥7) when the score is greater than or equal to the predetermined cutoff value or determining that the prostate cancer in the subject has a low Gleason score (≤6) when the score is less than the predetermined cutoff value.

Accordingly, the present disclosure also provides methods of determining whether a prostate cancer in a subject has a low Gleason score (≤6) or a high Gleason score (≥7), the method comprising: (a) determining the expression level of at least 24 biomarkers in a test sample from the subject, wherein the at least 24 biomarkers comprise AAMP, CHTOP, EDC4, FYCO1, HNRNPU, HPN, KRT23, MAN2B2, MAX, MRPS25, NDUFS2, PPRC1, RAD23A, REPIN1, SDR39U1, SETBP1, SLC18A2, SMC4, SPARC, SQLE, STRIP1, STX12, UNC45A, and XP (′, and a housekeeping gene; (b) normalizing the expression level of each of AAMP, CHTOP, EDC4, FYCO1, HNRNPU, HPN, KRT23, MAN2B2, MAX, MRPS25, NDUFS2, PPRC1, RAD23A, REPIN1, SDR39U1, SETBP1, SLC18A2, SMC4, SPARC, SQLE, STRIP1, STX12, UNC45A, and XPC to the expression level of the housekeeping gene, thereby obtaining a normalized expression level of each of AAMP, CHTOP, EDC4, FYCO1, HNRNPU, HPN, KRT23, MAN2B2, MAX, MRPS25, NDUFS2, PPRC1, RAD23A, REPIN1, SDR39U1, SETBP1, SLC18A2, SMC4, SPARC, SQLE, STRIP1, STX12, UNC45A, and XPC; (c) inputting each normalized expression level from step (b) into an algorithm to generate a score; (d) comparing the score to a predetermined cutoff value; and (e) determining that the prostate cancer in the subject has a high Gleason score (≥7) when the score is greater than the predetermined cutoff value or determining that the prostate cancer in the subject has a low Gleason score (≤6) when the score is less than or equal to the predetermined cutoff value.

In some aspects of the preceding methods, the predetermined cutoff value can be 50% on a scale of 0-100%.

Moreover, the present disclosure provides methods of determining the completeness of surgery in a subject having a prostate cancer, the method comprising: (a) determining the expression level of at least 24 biomarkers in a test sample from the subject after the surgery, wherein the 24 biomarkers comprise AAMP, CHTOP, EDC4, FYCO1, HNRNPU, HPN, KRT23, MAN2B2, MAX, MRPS25, NDUFS2, PPRC1, RAD23A, REPIN1, SDR39U1, SETBP1, SLC18A2, SMC4, SPARC, SQLE, STRIP1, STX12, UNC45A, XPC, and a housekeeping gene; (b) normalizing the expression level of each of AAMP, CHTOP, EDC4, FYCO1, HNRNPU, HPN, KRT23, MAN2B2, MAX, MRPS25, NDUFS2, PPRC1, RAD23A, REPIN1, SDR39U1, SETBP1, SLC18A2, SMC4, SPARC, SQLE, STRIP1, STX12, UNC45A, and XPC to the expression level of the housekeeping gene, thereby obtaining a normalized expression level of each of AAMP, CHTOP, EDC4, FYCO1, HNRNPU, HPN, KRT23, MAN2B2, MAX, MRPS25, NDUFS2, PPRC1, RAD23A, REPIN1, SDR39U1, SETBP1, SLC18A2, SMC4, SPARC, SQLE, STRIP1, STX12, UNC45A, and XPC; and (c) identifying that the prostate cancer is not completely removed or identifying that the prostate cancer is completely removed based on the normalized expression levels from step (b). In some aspects, identifying that the prostate cancer is not completely removed or identifying that the prostate cancer is completely removed based on the normalized expression levels from step (b) can comprise comparing the normalized expression levels to corresponding predetermined cutoff values and identifying that the prostate cancer is not completely removed or identifying that the prostate cancer is completely removed based on the relationship between the normalized expression levels and the corresponding predetermined cutoff values (e.g. greater than, greater than or equal to, less than, less than or equal to, or equal to).

Moreover, the present disclosure provides methods of determining the completeness of surgery in a subject having a prostate cancer, the method comprising: (a) determining the expression level of at least 24 biomarkers in a test sample from the subject after the surgery, wherein the 24 biomarkers comprise AAMP, CHTOP, EDC4, FYCO1, HNRNPU, HPN, KRT23, MAN2B2, MAX, MRPS25, NDUFS2, PPRC1, RAD23A, REPIN1, SDR39U1, SETBP1, SLC18A2, SMC4, SPARC, SQLE, STRIP1, STX12, UNC45A, XPC, and a housekeeping gene; (b) normalizing the expression level of each of AAMP, CHTOP, EDC4, FYCO1, HNRNPU, HPN, KRT23, MAN2B2, MAX, MRPS25, NDUFS2, PPRC1, RAD23A, REPIN1, SDR39U1, SETBP1, SLC18A2, SMC4, SPARC, SQLE, STRIP1, STX12, UNC45A, and XPC to the expression level of the housekeeping gene, thereby obtaining a normalized expression level of each of AAMP, CHTOP, EDC4, FYCO1, HNRNPU, HPN, KRT23, MAN2B2, MAX, MRPS25, NDUFS2, PPRC1, RAD23A, REPIN1, SDR39U1, SETBP1, SLC18A2, SMC4, SPARC, SQLE, STRIP1, STX12, UNC45A, and XPC; (c) inputting each normalized expression level from step (b) into an algorithm to generate a score; and (d) identifying that the prostate cancer is not completely removed or identifying that the neuroendocrine cancer is completely removed based on the score. In some aspects, identifying that the prostate cancer is not completely removed or identifying that the prostate cancer is completely removed based on the score can comprise comparing the score to a predetermined cutoff value and identifying that the prostate cancer is not completely removed or identifying that the neuroendocrine cancer is completely removed based on the relationship between the score and the corresponding predetermined cutoff value (e.g. greater than, greater than or equal to, less than, less than or equal to, or equal to).

Accordingly, the present disclosure provides methods of determining the completeness of surgery in a subject having a prostate cancer, the method comprising: (a) determining the expression level of at least 24 biomarkers in a test sample from the subject after the surgery, wherein the 24 biomarkers comprise AAMP, CHTOP, EDC4, FYCO1, HNRNPU, HPN, KRT23, MAN2B2, MAX, MRPS25, NDUFS2, PPRC1, RAD23A, REPIN1, SDR39U1, SETBP1, SLC18A2, SMC4, SPARC, SQLE, STRIP1, STX12, UNC45A, XPC, and a housekeeping gene; (b) normalizing the expression level of each of AAMP, CHTOP, EDC4, FYCO1, HNRNPU, HPN, KRT23, MAN2B2, MAX, MRPS25, NDUFS2, PPRC1, RAD23A, REPIN1, SDR39U1, SETBP1, SLC18A2, SMC4, SPARC, SQLE, STRIP1, STX12, UNC45A, and XPC to the expression level of the housekeeping gene, thereby obtaining a normalized expression level of each of AAMP, CHTOP, EDC4, FYCO1, HNRNPU, HPN, KRT23, MAN2B2, MAX, MRPS25, NDUFS2, PPRC1, RAD23A, REPIN1, SDR39U1, SETBP1, SLC18A2, SMC4, SPARC, SQLE, STRIP1, STX12, UNC45A, and XPC; (c) inputting each normalized expression level from step (b) into an algorithm to generate a score; (d) comparing the score to a predetermined cutoff value; and (e) identifying that the prostate cancer is not completely removed when the score is greater than or equal to the score or identifying that the prostate cancer is completely removed when the score is less than the predetermined cutoff value.

Accordingly, the present disclosure provides methods of determining the completeness of surgery in a subject having a prostate cancer, the method comprising: (a) determining the expression level of at least 24 biomarkers in a test sample from the subject after the surgery, wherein the 24 biomarkers comprise AAMP, CHTOP, EDC4, FYCO1, HNRNPU, HPN, KRT23, MAN2B2, MAX, MRPS25, NDUFS2, PPRC1, RAD23A, REPIN1, SDR39U1, SETBP1, SLC18A2, SMC4, SPARC, SQLE, STRIP1, STX12, UNC45A, XPC, and a housekeeping gene; (b) normalizing the expression level of each of AAMP, CHTOP, EDC4, FYCO1, HNRNPU, HPN, KRT23, MAN2B2, MAX, MRPS25, NDUFS2, PPRC1, RAD23A, REPIN1, SDR39U1, SETBP1, SLC18A2, SMC4, SPARC, SQLE, STRIP1, STX12, UNC45A, and XPC to the expression level of the housekeeping gene, thereby obtaining a normalized expression level of each of AAMP, CHTOP, EDC4, FYCO1, HNRNPU, HPN, KRT23, MAN2B2, MAX, MRPS25, NDUFS2, PPRC1, RAD23A, REPIN1, SDR39U1, SETBP1, SLC18A2, SMC4, SPARC, SQLE, STRIP1, STX12, UNC45A, and XPC; (c) inputting each normalized expression level from step (b) into an algorithm to generate a score; (d) comparing the score to a predetermined cutoff value; and (e) identifying that the prostate cancer is not completely removed when the score is greater than the score or identifying that the prostate cancer is completely removed when the score is less than or equal to the predetermined cutoff value.

In some aspects of the preceding methods, the predetermined cutoff value can be 50% on a scale of 0-100%.

The response of a subject having prostate cancer to a therapy can also be evaluated by comparing the scores determined by the same algorithm at different time points of the therapy. For example, the first time point can be prior to or after the administration of the therapy to the subject; the second time point is after the first time point and after the administration of the therapy to the subject. A first score is generated at the first time point, and a second score is generated at the second time point. When the second score is decreased as compared to the first score, the subject is considered to be responsive to the therapy. In some aspects, the second score is decreased as compared to the first score when the second score is at least 5% less than the first score, e.g., at least 10% less than the first score, at least 15% less than the first score, at least 25% less than the first score, at least 40% less than the first score, at least 50% less than the first score, at least 75% less than the first score, or at least 90% less than the first score. When the second score is not significantly decreased or has increased as compared to the first score, the subject is considered to be not responsive to the therapy.

The present disclosure also provides methods of evaluating the response of a subject having a prostate cancer to an anti-prostate cancer therapy, the method comprising: (a) at a first time point: (i) determining the expression level of at least 24 biomarkers in a test sample from the subject, wherein the at least 24 biomarkers comprise AAMP, CHTOP, EDC4, FYCO1, HNRNPU, HPN, KRT23, MAN2B2, MAX, MRPS25, NDUFS2, PPRC1, RAD23A, REPIN1, SDR39U1, SETBP1, SLC18A2, SMC4, SPARC, SQLE, STRIP1, STX12, UNC45A, XPC, and a housekeeping gene; (ii) normalizing the expression level of each of AAMP, CHTOP, EDC4, FYCO1, HNRNPU, HPN, KRT23, MAN2B2, MAX, MRPS25, NDUFS2, PPRC1, RAD23A, REPIN1, SDR39U1, SETBP1, SLC18A2, SMC4, SPARC, SQLE, STRIP1, STX12, UNC45A, and XPC to the expression level of the housekeeping gene, thereby obtaining a normalized expression level of each of AAMP, CHTOP, EDC4, FYCO1, HNRNPU, HPN, KRT23, MAN2B2, MAX, MRPS25, NDUFS2, PPRC1, RAD23A, REPIN1, SDR39U1, SETBP1, SLC18A2, SMC4, SPARC, SQLE, STRIP1, STX12, UNC45A, and XPC; (b) at a second time point, wherein the second time point is after the first time point and after the administration of the therapy to the subject: (i) determining the expression level of the at least 24 biomarkers in a test sample from the subject; (ii) normalizing the expression level of each of AAMP, CHTOP, EDC4, FYCO1, HNRNPU, HPN, KRT23, MAN2B2, MAX, MRPS25, NDUFS2, PPRC1, RAD23A, REPIN1, SDR39U1, SETBP1, SLC18A2, SMC4, SPARC, SQLE, STRIP1, STX12, UNC45A, and XPC to the expression level of the housekeeping gene, thereby obtaining a normalized expression level of each of AAMP, CHTOP, EDC4, FYCO1, HNRNPU, HPN, KRT23, MAN2B2, MAX, MRPS25, NDUFS2, PPRC1, RAD23A, REPIN1, SDR39U1, SETBP1, SLC18A2, SMC4, SPARC, SQLE, STRIP1, STX12, UNC45A, and XPC; (c) comparing the normalized expression levels from step (a)(ii) and step (b)(ii); and (d) identifying that the subject is responsive to the anti-prostate cancer therapy when the normalized expression levels from step (b)(ii) are decreased as compared to the expression levels from step (a)(ii) or identifying that the subject is not responsive to the anti-prostate cancer therapy when the normalized expression levels from step (b)(ii) are not decreased as compared to the normalized expression levels from step (a)(ii).

The present disclosure also provides methods of evaluating the response of a subject having a prostate cancer to an anti-prostate cancer therapy, the method comprising: (a) at a first time point: (i) determining the expression level of at least 24 biomarkers in a test sample from the subject, wherein the at least 24 biomarkers comprise AAMP, CHTOP, EDC4, FYCO1, HNRNPU, HPN, KRT23, MAN2B2, MAX, MRPS25, NDUFS2, PPRC1, RAD23A, REPIN1, SDR39U1, SETBP1, SLC18A2, SMC4, SPARC, SQLE, STRIP1, STX12, UNC45A, XPC, and a housekeeping gene; (ii) normalizing the expression level of each of AAMP, CHTOP, EDC4, FYCO1, HNRNPU, HPN, KRT23, MAN2B2, MAX, MRPS25, NDUFS2, PPRC1, RAD23A, REPIN1, SDR39U1, SETBP1, SLC18A2, SMC4, SPARC, SQLE, STRIP1, STX12, UNC45A, and XPC to the expression level of the housekeeping gene, thereby obtaining a normalized expression level of each of AAMP, CHTOP, EDC4, FYCO1, HNRNPU, HPN, KRT23, MAN2B2, MAX, MRPS25, NDUFS2, PPRC1, RAD23A, REPIN1, SDR39U1, SETBP1, SLC18A2, SMC4, SPARC, SQLE, STRIP1, STX12, UNC45A, and XPC; and (iii) inputting each normalized expression level from step (a)(ii) into an algorithm to generate a first score; (b) at a second time point, wherein the second time point is after the first time point and after the administration of the therapy to the subject: (i) determining the expression level of the at least 24 biomarkers in a test sample from the subject; (ii) normalizing the expression level of each of AAMP, CHTOP, EDC4, FYCO1, HNRNPU, HPN, KRT23, MAN2B2, MAX, MRPS25, NDUFS2, PPRC1, RAD23A, REPIN1, SDR39U1, SETBP1, SLC18A2, SMC4, SPARC, SQLE, STRIP1, STX12, UNC45A, and XPC to the expression level of the housekeeping gene, thereby obtaining a normalized expression level of each of AAMP, CHTOP, EDC4, FYCO1, HNRNPU, HPN, KRT23, MAN2B2, MAX, MRPS25, NDUFS2, PPRC1, RAD23A, REPIN1, SDR39U1, SETBP1, SLC18A2, SMC4, SPARC, SQLE, STRIP1, STX12, UNC45A, and XPC; and (iii) inputting each normalized expression level from step (b)(ii) into an algorithm to generate a second score; (c) comparing the first score and second score; and (d) identifying that the subject is responsive to the anti-prostate cancer therapy when the second score is decreased as compared to the first score or identifying that the subject is not responsive to the anti-prostate cancer therapy when the second score is not decreased as compared to the normalized expression levels from step (a)(ii).

### General Methods and Definitions

The following general methods and definitions can be applied to any of the preceding methods.

In some aspect, the test sample can comprise saliva.

Exemplary housekeeping genes include, but are not limited to, ATG4B, RHOA, TOX4, TPT1, and TXNIP. In some aspects, the housekeeping gene is TOX4.

Each of the biomarkers disclosed herein may have one or more transcript variants. The methods disclosed herein can measure the expression level of any one of the transcript variants for each biomarker.

In some aspects, determining the expression level of at least 24 biomarkers in a test sample from a subject can comprise contacting the test sample with a plurality of agents specific to detect the expression of the at least 24 biomarkers.

Accordingly, the present disclosure provides the use of a plurality of agents to detect the expression of at least 36 biomarkers in the manufacture of a kit for identifying the presence or absence of a prostate cancer by the methods described herein.

The present disclosure also provides the use of a plurality of agents to detect the expression of at least 36 biomarkers in the manufacture of a kit for identifying the risk of a subject having a prostate cancer by the methods described herein.

The present disclosure also provides the use of a plurality of agents to detect the expression of at least 36 biomarkers in the manufacture of a kit for determining whether a prostate cancer in a subject is stable or progressive by the methods described herein.

The present disclosure also provides the use of a plurality of agents to detect the expression of at least 36 biomarkers in the manufacture of a kit for determining whether a prostate cancer in a subject has a low Gleason score (≤6) or a high Gleason score (≥7) by the methods described herein.

The present disclosure also provides the use of a plurality of agents to detect the expression of at least 36 biomarkers in the manufacture of a kit for determining the completeness of a surgery to remove a prostate cancer in a subject by the methods described herein.

The present disclosure also provides the use of a plurality of agents to detect the expression of at least 36 biomarkers in the manufacture of a kit for evaluating the response of a subject having prostate cancer to an anti-prostate cancer therapy by the methods described herein.

The expression levels can be measured in a number of ways, including, but not limited to measuring the mRNA encoded by the selected genes; measuring the amount of protein encoded by the selected genes; measuring the activity of the protein encoded by the selected genes; or any combination thereof.

The biomarker can be RNA, cDNA, or protein. When the biomarker is RNA, the RNA can be reverse transcribed to produce cDNA (such as by RT-PCR), and the produced cDNA expression level is detected. The expression level of the biomarker can be detected by forming a complex between the biomarker and a labeled probe or primer. When the biomarker is RNA or cDNA, the RNA or cDNA is detected by forming a complex between the RNA or cDNA and a labeled nucleic acid probe or primer. The complex between the RNA or cDNA and the labeled nucleic acid probe or primer can be a hybridization complex.

As would be appreciated by the skilled artisan, gene expression can be detected by microarray analysis. Differential gene expression can also be identified or confirmed using the microarray technique. Thus, the expression profile biomarkers can be measured in either fresh or fixed tissue, using microarray technology. In this method, polynucleotide sequences of interest (including cDNAs and oligonucleotides) are plated, or arrayed, on a microchip substrate. The arrayed sequences are then hybridized with specific DNA probes from cells or tissues of interest. The source of mRNA typically is total RNA isolated from a biological sample, and corresponding normal tissues or cell lines may be used to determine differential expression.

In some aspects of the microarray technique, PCR amplified inserts of cDNA clones are applied to a substrate in a dense array. In some aspects, at least 10,000 nucleotide sequences are applied to the substrate. The microarrayed genes, immobilized on the microchip at 10,000 elements each, are suitable for hybridization under stringent conditions. Fluorescently labeled cDNA probes may be generated through incorporation of fluorescent nucleotides by reverse transcription of RNA extracted from tissues of interest. Labeled cDNA probes applied to the chip hybridize with specificity to each spot of DNA on the array. After stringent washing to remove non-specifically bound probes, the microarray chip is scanned by a device such as, confocal laser microscopy or by another detection method, such as a CCD camera. Quantitation of hybridization of each arrayed element allows for assessment of corresponding mRNA abundance. With dual color fluorescence, separately labeled cDNA probes generated from two sources of RNA are hybridized pair-wise to the array. The relative abundance of the transcripts from the two sources corresponding to each specified gene is thus determined simultaneously. Microarray analysis can be performed by commercially available equipment, following manufacturer's protocols.

In some aspects, the biomarkers (i.e. the SalivaPROSTest transcripts and/or housekeeping genes) can be detected in a saliva sample using RNAseq. As would be appreciated by the skilled artisan, the first step in gene expression profiling by RNAseq is extracting RNA from a saliva sample followed by the reverse transcription of the RNA template into cDNA to generate the RNA libraries. Sequencing adapters are added. cDNA is then sequenced using a sequencing platform. Data is analyzed and expressed as transcripts per million.

In some aspects, the biomarkers (i.e. the SalivaPROSTest transcripts and/or housekeeping genes) can be detected in a saliva sample using qRT-PCR. As would be appreciated by the skilled artisan, the first step in gene expression profiling by RT-PCR is extracting RNA from a biological sample followed by the reverse transcription of the RNA template into cDNA and amplification by a PCR reaction. The reverse transcription reaction step is generally primed using specific primers, random hexamers, or oligo-dT primers, depending on the goal of expression profiling. The two commonly used reverse transcriptases are avilo myeloblastosis virus reverse transcriptase (AMV-RT) and Moloney murine leukemia virus reverse transcriptase (MLV-RT).

In some aspects wherein the biomarker is protein, the protein can be detected by forming a complex between the protein and a labeled antibody. The label can be any label for example a fluorescent label, chemiluminescence label, radioactive label, etc. Exemplary methods for protein detection include, but are not limited to, enzyme immunoassay (EIA), radioimmunoassay (RIA), Western blot analysis and enzyme linked immunoabsorbant assay (ELISA). For example, the biomarker can be detected in an ELISA, in which the biomarker antibody is bound to a solid phase and an enzyme-antibody conjugate is employed to detect and/or quantify biomarker present in a sample. Alternatively, a western blot assay can be used in which solubilized and separated biomarker is bound to nitrocellulose paper. The combination of a highly specific, stable liquid conjugate with a sensitive chromogenic substrate allows rapid and accurate identification of samples.

In some aspects, the methods described herein can have a specificity, sensitivity, and/or accuracy of at least 75%, 80%, 85%, 90%, 91%, 92%, 93%, 94%, 95%, 96%, 97%, 98%, or 99%.

In some aspects, the methods described herein can have a specificity (e.g. a specificity for identifying the presence or absence of prostate cancer, a specificity for identifying whether a prostate cancer is stable or progressive, a specificity for identifying the completeness of surgery in a subject having prostate cancer, or a specificity for evaluating the response of a subject having prostate cancer to an anti-prostate cancer therapy) of at least 75%, 80%, 85%, 90%, 91%, 92%, 93%, 94%, 95%, 96%, 97%, 98%, or 99%.

In some aspects, the methods described herein can have a sensitivity (e.g. a specificity for identifying the presence or absence of prostate cancer, a specificity for identifying whether a prostate cancer is stable or progressive, a specificity for identifying the completeness of surgery in a subject having prostate cancer, or a specificity for evaluating the response of a subject having prostate cancer to an anti-prostate cancer therapy) of at least 75%, 80%, 85%, 90%, 91%, 92%, 93%, 94%, 95%, 96%, 97%, 98%, or 99%.

In some aspects, the methods described herein can have a accuracy (e.g. a specificity for identifying the presence or absence of prostate cancer, a specificity for identifying whether a prostate cancer is stable or progressive, a specificity for identifying the completeness of surgery in a subject having prostate cancer, or a specificity for evaluating the response of a subject having prostate cancer to an anti-prostate cancer therapy) of at least 75%, 80%, 85%, 90%, 91%, 92%, 93%, 94%, 95%, 96%, 97%, 98%, or 99%.

Any algorithm that can generate a score for a sample by assessing where that sample value falls onto a prediction model generated using different techniques, e.g., decision trees, can be used in the methods disclosed herein. The algorithm analyzes the data (i.e., expression levels) and then assigns a score. In some aspects, the algorithm can be a machine-learning algorithm. Exemplary algorithms that can be used in the methods disclosed herein can include, but are not limited to, XGB, RF, glmnet, cforest, CART, treebag, knn, nnet, SVM-radial, SVM-linear, NB, and mlp. In some aspects, the algorithm can be XGB (also called XGBoost). XGB is an implementation of gradient boosted decision trees designed for speed and performance. In some aspects, the algorithm can be Random Forest. In some aspects, the Random Forest algorithm can be a grid-search optimized Random-Forest. Random Forest is an implementation of an ensemble learning method for classification, regression and other tasks that operates by constructing a multitude of decision trees at training time.

In some aspects of the methods of the present disclosure, the machine learning algorithm can be trained using: a) the expression levels or normalized expression levels of the at least 24 biomarkers in at least one biological sample (e.g. saliva) from at least one subject who does not have prostate cancer; and b) the expression levels or normalized expression levels of the at least 24 biomarkers in at least one biological sample (e.g. saliva) from at least one subject who has prostate cancer. That is, in some aspects, the machine learning algorithm is trained using the expression levels or normalized expression levels of the at least 24 biomarkers obtained from a plurality of reference samples obtained from subjects not having prostate cancer and the expression levels or normalized expression levels of the at least 24 biomarkers from a plurality of reference samples obtained from subjects having prostate cancer.

In some aspects, one or more predetermined cutoff values can be derived from a plurality of reference samples obtained from subjects not having or not diagnosed with a neoplastic disease. The plurality of reference samples can be about 2 to about 500 samples, about 2 to about 200 samples, about 10 to about 100, or about 20 to about 80 samples.

In some aspects, determining a predetermined cutoff value can comprise inputting the normalized expression level of the SalivaPROSTest transcripts from each reference sample into the same algorithm used in the methods described above, thereby generating a plurality of scores from the plurality of reference samples. The predetermined cutoff value can then be determined by taking the arithmetic mean of these scores. In some aspects, the reference samples can comprise saliva. In some aspects, the reference sample is of the same type as the test sample.

In some aspects of the methods of the present disclosure, a predetermined cutoff value can be calculated and/or selected using at least one receiver operating characteristic (ROC) curve. In some aspects of the methods of the present disclosure, a predetermined cutoff value can be calculated and/or selected to have any of the features described herein (e.g., a specific sensitivity, specificity, accuracy, or any combination thereof) using any method known in the art, as would be appreciated by the skilled artisan.

In some aspects, the methods described herein can further comprise treating a subject with an anti-prostate cancer therapy.

Accordingly, in some aspects, the methods described herein further comprise treating a subject identified as having prostate cancer with an anti-prostate cancer therapy. In some aspects, the methods described herein further comprise treating a subject identified as having a progressive prostate cancer with at least one anti-prostate cancer therapy. In some aspects, the methods described herein further comprise treating a subject identified as having a high risk of prostate cancer with at least one anti-prostate cancer therapy. In some aspects, the methods described herein further comprise treating a subject whose prostate cancer has not been completely removed by surgery with at least one anti-prostate cancer therapy.

In some aspects, the methods described further comprise treating a subject who is identified as not responding to an anti-prostate cancer therapy with a different anti-prostate cancer therapy. In some aspects, the methods described further comprise continuing to treat a subject who is identified as responding to an anti-prostate cancer therapy with the same anti-prostate cancer therapy.

In some aspects, anti-prostate cancer therapy can comprise active surveillance, surgery, radiation therapy, cryotherapy, hormone therapy, chemotherapy, vaccine treatment, bone-directed treatment, or any combination thereof. Anti-prostate cancer therapy can comprise any therapeutic known in the art that is effective at treating neuroendocrine cancer.

As would be appreciated by the skilled artisan, active surveillance can comprise a doctor visit with a prostate-specific antigen blood test and digital rectal exam about every 6 months. As would be appreciated by the skilled artisan, active surveillance can also comprise prostate biopsies, which may be done every year.

As would be appreciated by the skilled artisan, surgery for prostate cancer patients can comprise a radical prostatectomy.

As would be appreciated by the skilled artisan, radiation therapy for prostate cancer can comprise external beam radiation, brachytherapy and radiopharmaceuticals. As would be appreciated by the skilled artisan, radiopharmaceuticals can comprise 177Lu-PSMA.

As would be appreciated by the skilled artisan, cryotherapy (also called cryosurgery or cryoablation) can comprise the use of very cold temperatures to freeze and kill prostate cancer cells.

As would be appreciated by the skilled artisan, hormone therapy is also called androgen deprivation therapy or androgen suppression therapy. Without wishing to be bound by theory, the goal is to reduce levels of male hormones, called androgens, in the body, or to stop them from affecting prostate cancer cells. Hormone therapy can comprise orchiectomy. Hormone therapy can comprise administration of a compound that lowers the level of androgens, including, but not limited to, Luteinizing hormone-releasing hormone (LHRH) agonists, LHRH antagonists, and CYP17 inhibitors. Known LHRH agonists include, but are not limited to, leuprolide, goserelin, triptorelin, and histrelin. Known LHRH antagonists include degarelix. Known CYP17 inhibitors include abiraterone. Hormone therapy can also comprise administration of an anti-androgen, including, but not limited to, flutamide, bicalutamide, nilutamide, and enzalutamide. Hormone therapy can comprise administration of an androgen-suppressing drug, including, but not limited to, estrogens and ketoconazole.

As would be appreciated by the skilled artisan, chemotherapy can comprise docetaxel, cabazitaxel, mitoxantrone, estramustine, or any combination thereof.

As would be appreciated by the skilled artisan, vaccine treatment can comprise Sipuleucel-T.

As would be appreciated by the skilled artisan, if a prostate cancer has grown outside the prostate, preventing or slowing the spread of the cancer to the bones is a major goal of treatment. Bone-directed treatment can include bisphosphonates (e.g., zoledronic acid), denosumab, corticosteroids, external radiation therapy, radiopharmaceuticals (e.g., Strontium-89, Samarium-153, Lutetium-177 or Radium-223), and pain medicines.

The sequence information of the prostate cancer biomarkers and housekeeping genes is shown in Table 1. Table 1 shows representative sequences for each of the prostate cancer biomarkers and housekeeping genes discussed herein. The skilled artisan would appreciate that in addition to the specific sequences shown in Table 1, other isoforms and variants of the prostate cancer biomarkers can be measured in the methods of the present disclosure to obtain an expression level of the biomarker or housekeeping gene.

TABLE 1
Prostate Cancer Biomarker/Housekeeper Sequence Information

| Gene Name | RefSeq Accession | SEQ ID NO: |
|---|---|---|
| AAMP | NM_001087.4 | 1 |
| CHTOP | NM_001206612.1 | 2 |
| EDC4 | NM_014329.4 | 3 |
| FYCO1 | NM_024513.3 | 4 |
| HNRNPU | NM_004501.3 | 5 |
| HPN | NM_002151.2 | 6 |
| KRT23 | NM_001282433.1 | 7 |
| MAN2B2 | NM_001292038.1 | 8 |
| MAX | NM_001320415.1 | 9 |
| MRPS25 | NM_022497.4 | 10 |
| NDUFS2 | NM_001166159.1 | 11 |
| PPRC1 | NM_001288727.1 | 12 |
| RAD23A | NM_001270362.1 | 13 |
| REPIN1 | NM_013400.3 | 14 |
| SDR39U1 | NM_020195.2 | 15 |
| SETBP1 | NM_001130110.1 | 16 |
| SLC18A2 | NM_003054.4 | 17 |
| SMC4 | NM_001002800.2 | 18 |
| SPARC | NM_001309443.1 | 19 |
| SQLE | NM_003129.3 | 20 |
| STRIP1 | NM_001270768.1 | 21 |
| STX12 | NM_177424.2 | 22 |
| UNC45A | NM_001039675.1 | 23 |
| XPC | NM_004628.4 | 24 |
| ATG4B | NM_178326.3 | 25 |
| RHOA | NM_001313941.2 | 26 |
| TOX4 | NM_001303523.2 | 27 |
| TPT1 | NM_001286272.2 | 28 |
| TXNIP | NM_001313972.2 | 29 |

### Definitions

The articles “a” and “an” are used in this disclosure to refer to one or more than one (i.e., to at least one) of the grammatical object of the article. By way of example, “an element” means one element or more than one element.

The term “and/or” is used in this disclosure to mean either “and” or “or” unless indicated otherwise.

As used herein, the terms “polynucleotide” and “nucleic acid molecule” are used interchangeably to mean a polymeric form of nucleotides of at least 10 bases or base pairs in length, either ribonucleotides or deoxynucleotides or a modified form of either type of nucleotide, and is meant to include single and double stranded forms of DNA. As used herein, a nucleic acid molecule or nucleic acid sequence that serves as a probe in a microarray analysis preferably comprises a chain of nucleotides, more preferably DNA and/or RNA. In some aspects, a nucleic acid molecule or nucleic acid sequence comprises other kinds of nucleic acid structures such as for instance a DNA/RNA helix, peptide nucleic acid (PNA), locked nucleic acid (LNA) and/or a ribozyme. Hence, as used herein the term “nucleic acid molecule” also encompasses a chain comprising non-natural nucleotides, modified nucleotides and/or non-nucleotide building blocks which exhibit the same function as natural nucleotides.

As used herein, the terms “hybridize,” “hybridizing”, “hybridizes,” and the like, used in the context of polynucleotides, are meant to refer to conventional hybridization conditions, such as hybridization in 50% formamide/6×SSC/0.1% SDS/100 μg/ml ssDNA, in which temperatures for hybridization are above 37 degrees centigrade and temperatures for washing in 0.1×SSC/0.1% SDS are above 55 degrees C., and preferably to stringent hybridization conditions.

As used herein, the term “normalization” or “normalizer” refers to the expression of a differential value in terms of a standard value to adjust for effects which arise from technical variation due to sample handling, sample preparation, and measurement methods rather than biological variation of biomarker concentration in a sample. For example, when measuring the expression of a differentially expressed protein, the absolute value for the expression of the protein can be expressed in terms of an absolute value for the expression of a standard protein that is substantially constant in expression.

The terms “diagnosis” and “diagnostics” also encompass the terms “prognosis” and “prognostics”, respectively, as well as the applications of such procedures over two or more time points to monitor the diagnosis and/or prognosis over time, and statistical modeling based thereupon. Furthermore, the term diagnosis includes: a. prediction (determining if a patient will likely develop aggressive disease (hyperproliferative/invasive)), b. prognosis (predicting whether a patient will likely have a better or worse outcome at a pre-selected time in the future), c. therapy selection, d. therapeutic drug monitoring, and e. relapse monitoring.

“Accuracy” refers to the degree of conformity of a measured or calculated quantity (a test reported value) to its actual (or true) value. Clinical accuracy relates to the proportion of true outcomes (true positives (TP) or true negatives (TN)) versus misclassified outcomes (false positives (FP) or false negatives (FN)), and may be stated as a sensitivity, specificity, positive predictive values (PPV) or negative predictive values (NPV), or as a likelihood, odds ratio, among other measures.

The term “biological sample” as used herein refers to any sample of biological origin potentially containing one or more biomarkers. Examples of biological samples include tissue, organs, or bodily fluids such as whole blood, plasma, serum, tissue, lavage or any other specimen used for detection of disease.

The term “subject” as used herein refers to a mammal, preferably a human. In some aspects, the subject has at least one prostate cancer symptom. In some aspects, the subject has a predisposition or familial history for developing a prostate cancer. The subject could also be previously diagnosed with a prostate cancer and is tested for cancer recurrence. In some aspects, the subject has benign prostate hyperplasia.

“Treating” or treatment of a disease or condition refers to executing a protocol or treatment plan, which may include administering one or more therapeutic agents to a patient, in an effort to alleviate signs or symptoms of the disease or the recurrence of the disease. Desirable effects of treatment include decreasing the rate of disease progression, ameliorating or palliating the disease state, and remission, increased survival, improved quality of life or improved prognosis. In addition, “treating” or “treatment” does not require complete alleviation of signs or symptoms, does not require a cure, and specifically includes protocols or treatment plans that have only a marginal effect on the patient.

As used herein, “prevent”, “preventing” and the like describe stopping the onset of the disease, condition or disorder, or one or more symptoms or complications thereof.

Biomarker levels may change due to treatment of the disease. The changes in biomarker levels may be measured by the present disclosure. Changes in biomarker levels may be used to monitor the progression of disease or therapy.

“Altered”, “changed” or “significantly different” refer to a detectable change or difference from a reasonably comparable state, profile, measurement, or the like. Such changes may be all or none. They may be incremental and need not be linear. They may be by orders of magnitude. A change may be an increase or decrease by 5%, 10%, 20%, 30%, 40%, 50%, 60%, 70%, 80%, 90%, 95%, 99%, 100%, or more, or any value in between 0% and 100%. Alternatively, the change may be 1-fold, 1.5-fold, 2-fold, 3-fold, 4-fold, 5-fold or more, or any values in between 1-fold and 5-fold. The change may be statistically significant with a p value of 0.1, 0.05, 0.001, or 0.0001.

The term “stable disease” refers to a diagnosis for the presence of a prostate cancer, however the prostate cancer has been treated and remains in a stable condition, i.e. one that that is not progressive, as determined by imaging data and/or best clinical judgment.

The term “progressive disease” refers to a diagnosis for the presence of a highly active state of a prostate cancer, i.e., one has not been treated and is not stable or has been treated and has not responded to therapy, or has been treated and active disease remains, as determined by imaging data and/or best clinical judgment.

The term “neoplastic disease” refers to any abnormal growth of cells or tissues being either benign (non-cancerous) or malignant (cancerous). For example, the neoplastic disease can be a prostate cancer.

The term “neoplastic tissue” refers to a mass of cells that grow abnormally. The term “non-neoplastic tissue” refers to a mass of cells that grow normally.

As used herein, the term “about” when used in conjunction with numerical values and/or ranges generally refers to those numerical values and/or ranges near to a recited numerical value and/or range. In some instances, the term “about” can mean within +10% of the recited value. For example, in some instances, “about 100 [units]” can mean within +10% of 100 (e.g., from 90 to 110).

### EXAMPLES

The disclosure is further illustrated by the following examples, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure and/or scope of the appended claims.

### Example 1. Derivation of a 24-Marker Gene Panel

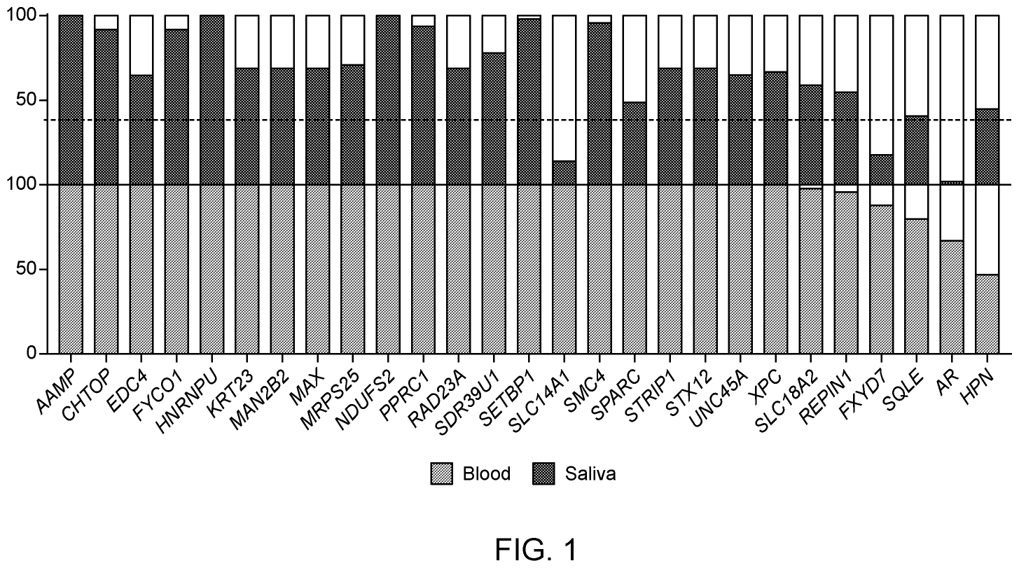

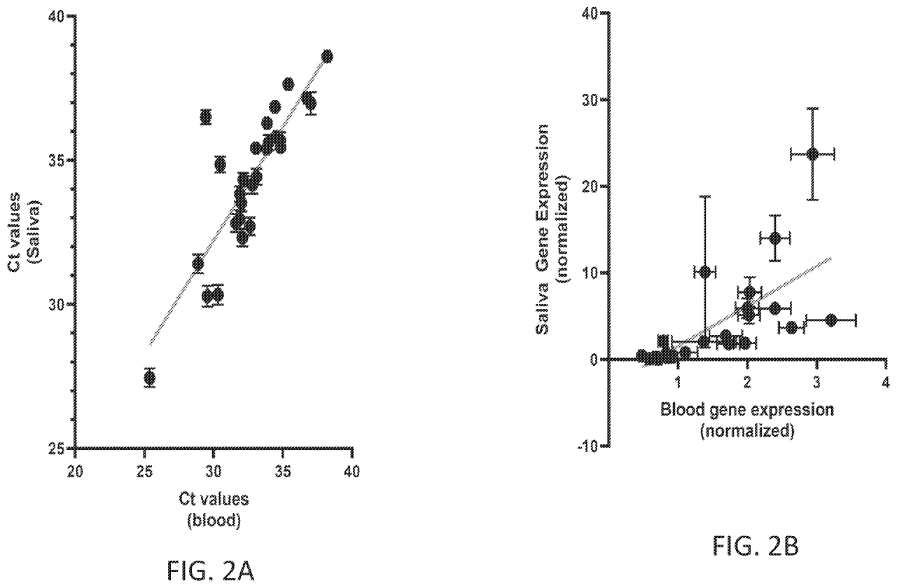

panel has previously been developed and patented for blood evaluation. This comprises 38 marker genes. The panel of SalivaPROSTest transcripts was derived from evaluating gene expression in matched blood and saliva samples collected from 51 prostate cancer patients, including the expression of biomarkers previously identified in blood samples from prostate cancer patients (see US 2019-0259471A1). All of the previously identified genes were detectable in blood but only 24 of these were detectable in >40% of saliva samples (FIG. 1). These 24 genes were highly correlated both in terms of measurement (Ct values) as well as when expressed as normalized values. The correlation between blood and saliva Ct values was r=0.85 (p<0.0001, FIG. 2A) and for normalized values the Pearson r value was 0.67 (p=0.0003; FIG. 2B).

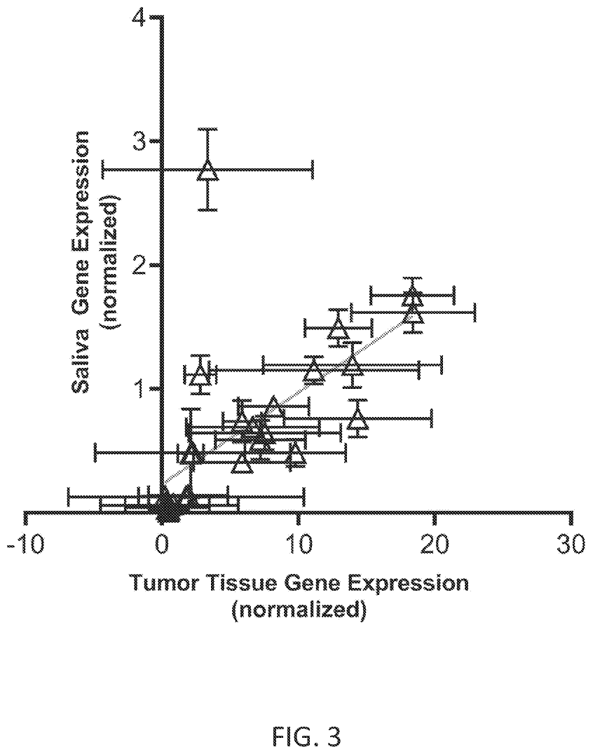

These genes were demonstrated to be highly expressed in prostate cancer tumor tissue and there was a significant correlation (r=0.64, p=0.0004) with saliva gene expression identifying that saliva could be used to effectively function as a liquid biopsy (FIG. 3).

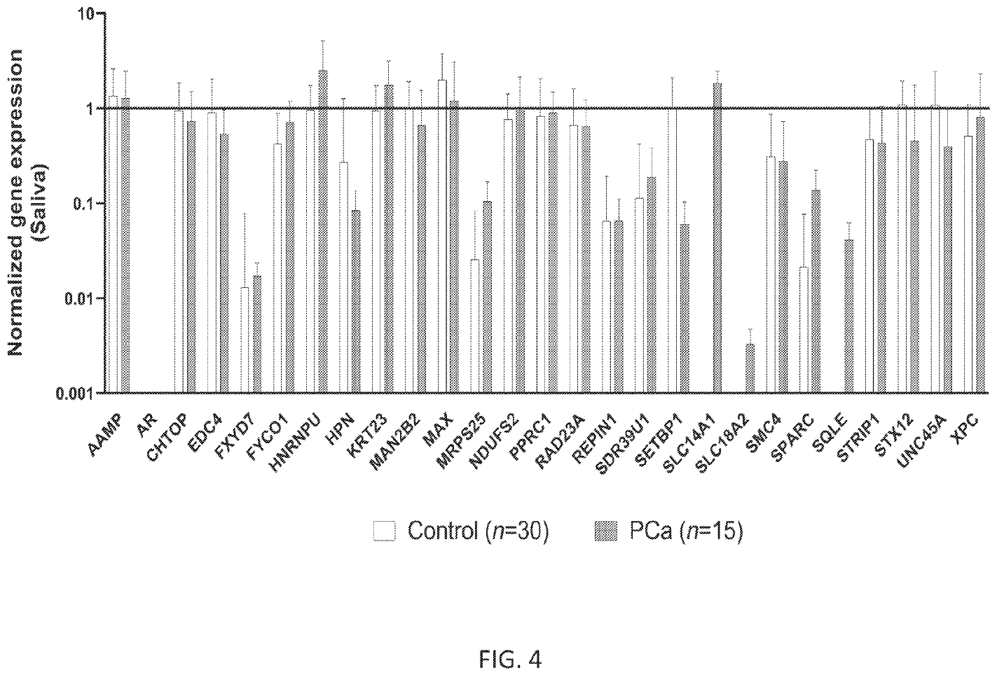

Evaluation of transcripts in a preliminary dataset of saliva samples from age (average 76 years) matched prostate cancers (n=15) and normal saliva (n=30) confirmed expression of the 24 genes as markers of prostate cancer (FIG. 4). These data demonstrate the candidate target transcripts are produced by neoplastic transformed prostate cells and are detectable in saliva.

An artificial intelligence model of prostate cancer disease was built using normalized gene expression of these 24 markers in saliva from Controls (n=163), and PCa (n=51) samples. The dataset was randomly split into training and testing partitions for model creation and validation respectively. Twelve algorithms were evaluated (XGB, RF, glmnet, cforest, CART, treebag, knn, nnet, SVM-radial, SVM-linear, NB and mlp). The top performing algorithm (RF—“random forest”) best predicted the training data. In the test set, RF produced probability scores that predicted the sample. Each probability score reflects the “certainty” of an algorithm that an unknown sample belongs to either “Control” or “PCa” class. For example, an unknown sample S1 can have the following probability vector [Control=20%, PCa=80%]. This sample would be considered a PCa sample.

TABLE 2
24 PCa marker gene panel (not including the housekeeping gene)

| | | NCBI | | | | | |
|---|---|---|---|---|---|---|---|
| Prostate Cancer Biomarker Gene | | Chromosome | | | Exon | Assay | Amplicon |
| Symbol | Name | Location | UniGene ID | RefSeq | Boundary | Location | Length |
| AAMP | Angio-associated | Chr. 2: | Hs.83347 | NM_001087.4 | 2-3 | 395 | 58 |
| | migratory cell protein | 218264129- | | | | | |
| | | 218270209 | | | | | |
| CHTOP | chromatin target of | Chr. 1: | Hs.611057 | NM_001206612.1 | 2-3 | 445 | 66 |
| | PRMT1 | 153633982- | | | | | |
| | | 153646306 | | | | | |
| EDC4 | enhancer of mRNA | Chr. 16: | Hs.75682 | NM_014329.4 | 27-28 | 4089 | 72 |
| | decapping 4 | 67873023- | | | | | |
| | | 67884514 | | | | | |
| FYCO1 | FYVE and coiled-coil | Chr. 3: | Hs.200227 | NM_024513.3 | 11-12 | 3651 | 59 |
| | domain containing 1 | 45917899- | | | | | |
| | | 45995824 | | | | | |
| HNRNPU | heterogeneous | Chr. 1: | Hs.106212 | NM_004501.3 | 8-9 | 1773 | 79 |
| | nuclear | 244842123- | | | | | |
| | ribonucleoprotein U | 244864720 | | | | | |
| HPN | hepsin | Chr. 19: | Hs.182385 | NM_002151.2 | 10-11 | 1052 | 89 |
| | | 35040506- | | | | | |
| | | 35066573 | | | | | |
| KRT23 | keratin 23 | Chr. 17: | Hs.9029 | NM_001282433.1 | 5-6 | 1045 | 90 |
| | | 40922696- | | | | | |
| | | 40937643 | | | | | |
| MAN2B2 | mannosidase alpha | Chr. 4: | Hs.188464 | NM_001292038.1 | 5-6 | 716 | 55 |
| | class 2B member 2 | 6575174- | | | | | |
| | | 6622403 | | | | | |
| MAX | MYC associated | Chr. 14: | Hs.285354 | NM_001320415.1 | 3-4 | 377 | 61 |
| | factor X | 65006101- | | | | | |
| | | 65102695 | | | | | |
| MRPS25 | mitochondrial | Chr. 3: | Hs.657764 | NM_022497.4 | 2-3 | 380 | 80 |
| | ribosomal protein | 15042251- | | | | | |
| | S25 | 15065337 | | | | | |
| NDUFS2 | NADH: ubiquinone | Chr. 1: | Hs.173611 | NM_001166159.1 | 3-4 | 632 | 80 |
| | oxidoreductase core | 161197377- | | | | | |
| | subunit S2 | 161214395 | | | | | |
| PPRC1 | peroxisome | Chr. 10: | Hs.533551 | NM_001288727.1 | 1-2 | 224 | 55 |
| | proliferator-activated | 102132994- | | | | | |
| | receptor gamma, | 102150333 | | | | | |
| | coactivator-related 1 | | | | | | |
| RAD23A | RAD23 homolog A, | Chr. 19: | Hs.643267 | NM_001270362.1 | 1-2 | 203 | 74 |
| | nucleotide excision | 12945814- | | | | | |
| | repair protein | 12953643 | | | | | |
| REPIN1 | replication initiator 1 | Chr. 7: | Hs.647086 | NM_013400.3 | 3-4 | 474 | 70 |
| | | 150368228- | | | | | |
| | | 150374044 | | | | | |
| SDR39U1 | short chain | Chr. 14: | Hs.643552 | NM_020195.2 | 4-5 | 358 | 91 |
| | dehydrogenase/ | 24439766- | | | | | |
| | reductase family | 24442905 | | | | | |
| | 39U member 1 | | | | | | |
| SETBP1 | SET binding protein 1 | Chr. 18: | Hs.435458 | NM_001130110.1 | 2-3 | 882 | 70 |
| | | 44680173- | | | | | |
| | | 45068510 | | | | | |
| SLC18A2 | solute carrier family | Chr. 10: | Hs.596992 | NM_003054.4 | 15-16 | 1605 | 145 |
| | 18 member A2 | 117241073- | | | | | |
| | | 117279430 | | | | | |
| SMC4 | structural | Chr. 3: | Hs.58992 | NM_001002800.2 | 5-6 | 1134 | 91 |
| | maintenance of | 160399304- | | | | | |
| | chromosomes 4 | 160434962 | | | | | |
| SPARC | secreted protein | Chr. 5: | Hs.111779 | NM_001309443.1 | 6-7 | 650 | 76 |
| | acidic and cysteine | 151661096- | | | | | |
| | rich | 151687054 | | | | | |
| SQLE | squalene epoxidase | Chr. 8: | Hs.71465 | NM_003129.3 | 9-10 | 2368 | 109 |
| | | 124998478- | | | | | |
| | | 125022283 | | | | | |
| STRIP1 | striatin interacting | Chr. 1: | Hs.584996 | NM_001270768.1 | 12-13 | 1303 | 64 |
| | protein 1 | 110031577- | | | | | |
| | | 110054641 | | | | | |
| STX12 | syntaxin 12 | Chr. 1: | Hs.523855 | NM_177424.2 | 1-2 | 248 | 70 |
| | | 27773183- | | | | | |
| | | 27824452 | | | | | |
| UNC45A | unc-45 myosin | Chr. 15: | Hs.389461 | NM_001039675.1 | 20-21 | 3095 | 62 |
| | chaperone A | 90929980- | | | | | |
| | | 90954093 | | | | | |
| XPC | XPC complex | Chr. 3: | Hs.475538 | NM_004628.4 | 2-3 | 403 | 104 |
| | subunit, DNA | 14145147- | | | | | |
| | damage recognition | 14178672 | | | | | |
| | and repair factor | | | | | | |

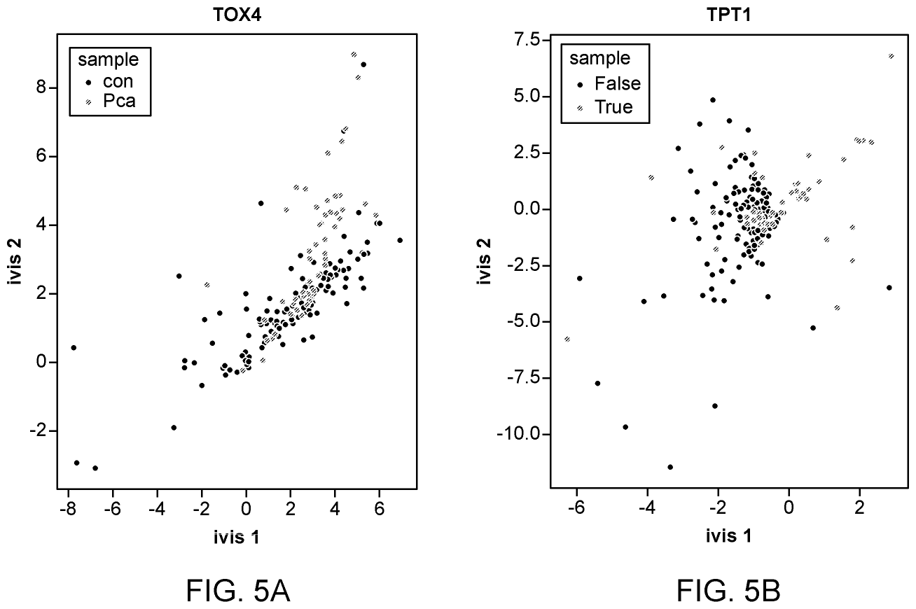

The 24 marker genes identified by the Random Forest algorithm were visualized in the derivation cohort of n=163 control samples and 51 cancer samples using the IVIS algorithm (FIG. 5A-B).

### Example 2. Clinical Utility

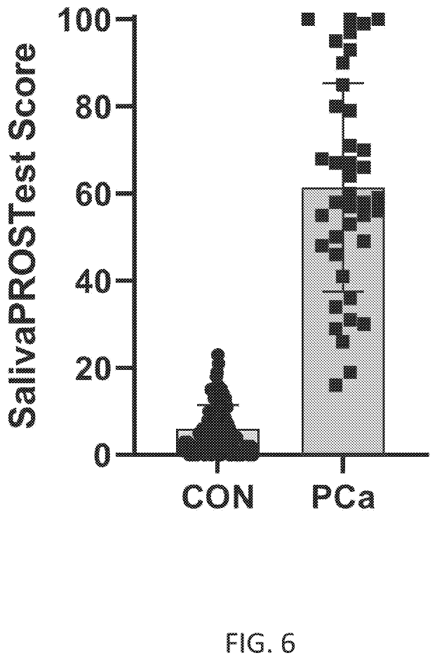

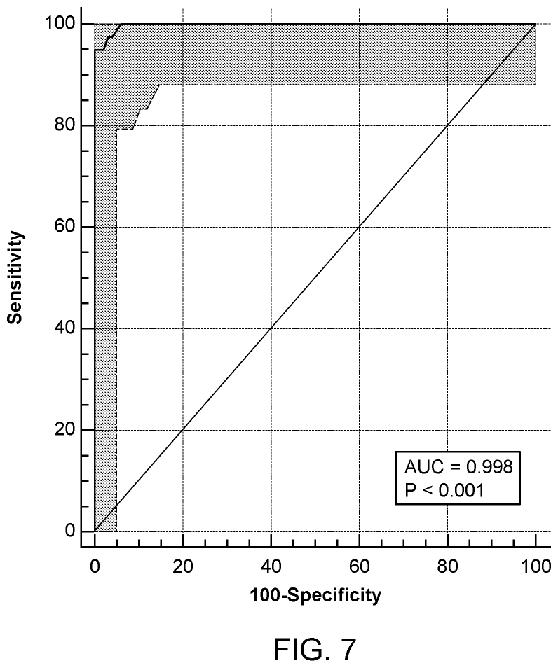

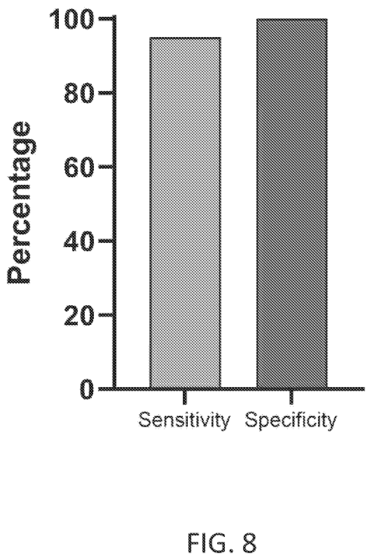

The SalivaPROSTest scores were significantly (p<0.001) elevated in PCa (61±24%) compared to control men including those with benign prostate hyperplasia (8±9%)(FIG. 6). The data (receiver operator curve analysis and metrics) for the utility of the test to differentiate patients with prostate cancer (n=40) from controls (n=100) in the validation is included in FIG. 7. The score exhibited an area under the curve (AUROC) of 0.99. The metrics are: sensitivity: 95% and specificity: 100% (FIG. 8). The Youden index J is 0.95 and the Z-statistic for differentiating non-malignant prostate disease and controls was 304.7.

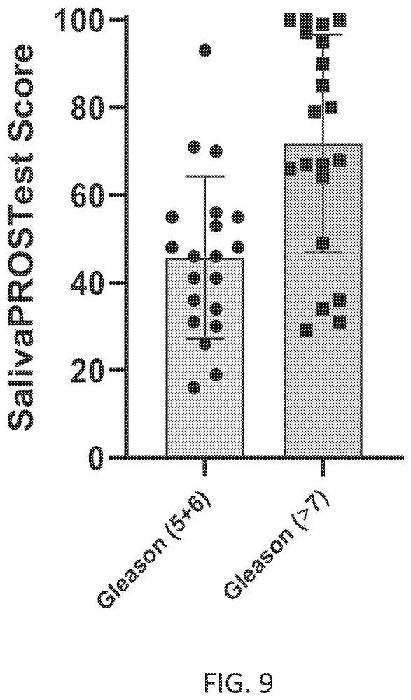

The SalivaPROSTest scores were significantly (p<0.002) elevated in high grade (Gleason score ≥7: 72±25%) compared to low grade (Gleason 5+6) PCa (46±19%). The data is included in FIG. 9.

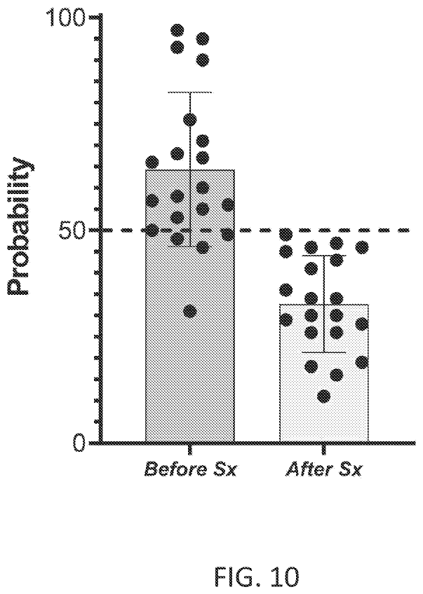

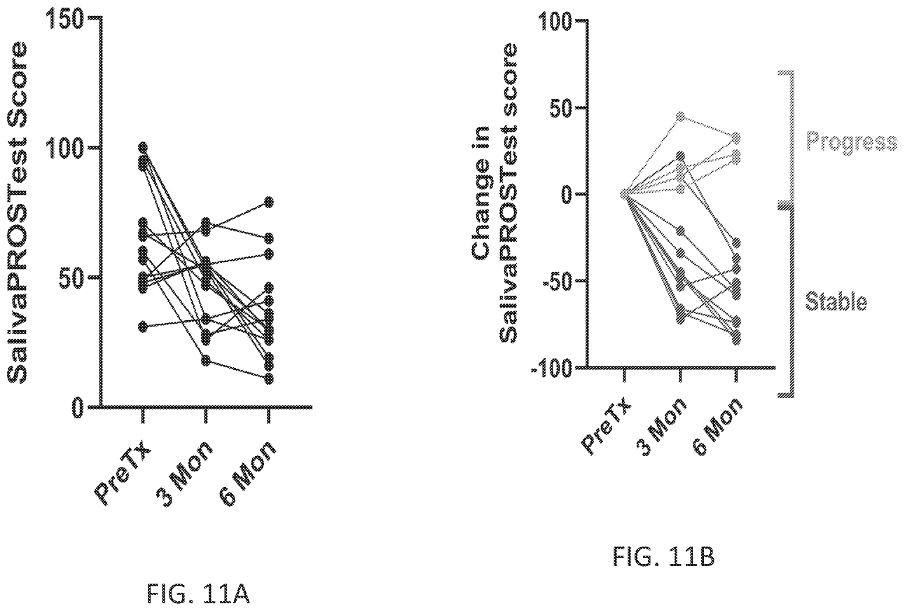

Specific evaluation of a prostate carcinoma cohort before and after surgery identified that complete removal of a tumor and no evidence of disease was associated with a significant decrease (p<0.0001) in the SalivaPROSTest score (FIG. 10). Levels were not significantly different to controls. Evaluation of separate cohort identified that patients who underwent and responded to therapy exhibited a significant lower score (p<0.001) that those diagnosed with disease (FIG. 11). Therapies included ADT and 177Lu-PSMA therapy. The tool can therefore accurately identify treatment responses in prostate cancer disease.

### EQUIVALENTS

While the present inventions have been described in conjunction with the specific embodiments set forth above, many alternatives, modifications and other variations thereof will be apparent to those of ordinary skill in the art. All such alternatives, modifications and variations are intended to fall within the spirit and scope of the present inventions.

What is claimed is:

1. A method of identifying the presence or absence of prostate cancer in a subject in need thereof, the method comprising:
(a) determining the expression level of at least 24 biomarkers in a saliva sample from the subject, wherein the at least 24 biomarkers comprise AAMP, CHTOP, EDC4, FYCO1, HNRNPU, HPN, KRT23, MAN2B2, MAX, MRPS25, NDUFS2, PPRC1, RAD23A, REPIN1, SDR39U1, SETBP1, SLC18A2, SMC4, SPARC, SQLE, STRIP1, STX12, UNC45A, XPC, and a housekeeping gene;
(b) normalizing the expression level of each of AAMP, CHTOP, EDC4, FYCO1, HNRNPU, HPN, KRT23, MAN2B2, MAX, MRPS25, NDUFS2, PPRC1, RAD23A, REPIN1, SDR39U1, SETBP1, SLC18A2, SMC4, SPARC, SQLE, STRIP1, STX12, UNC45A, and XPC to the expression level of the housekeeping gene, thereby obtaining a normalized expression level of each of AAMP, CHTOP, EDC4, FYCO1, HNRNPU, HPN, KRT23, MAN2B2, MAX, MRPS25, NDUFS2, PPRC1, RAD23A, REPIN1, SDR39U1, SETBP1, SLC18A2, SMC4, SPARC, SQLE, STRIP1, STX12, UNC45A, and XPC;
(c) inputting each normalized expression from step (b) into an algorithm to generate a score;
(d) comparing the score to a predetermined cutoff value; and
(e) identifying the presence of prostate cancer in the subject when the score is greater than or equal to the predetermined cutoff value or determining the absence of prostate cancer in the subject when the score is less than the predetermined cutoff value.

2. The method of claim 1, wherein the predetermined cutoff value is 23% on a scale of 0-100%.

3. A method of determining whether a prostate cancer in a subject is stable or progressive, the method comprising:
(a) determining the expression level of at least 24 biomarkers in a saliva sample from the subject, wherein the at least 24 biomarkers comprise AAMP, CHTOP, EDC4, FYCO1, HNRNPU, HPN, KRT23, MAN2B2, MAX, MRPS25, NDUFS2, PPRC1, RAD23A, REPIN1, SDR39U1, SETBP1, SLC18A2, SMC4, SPARC, SOLE, STRIP1, STX12, UNC45A, XPC, and a housekeeping gene;
(b) normalizing the expression level of each of AAMP, CHTOP, EDC4, FYCO1, HNRNPU, HPN, KRT23, MAN2B2, MAX, MRPS25, NDUFS2, PPRC1, RAD23A, REPIN1, SDR39U1, SETBP1, SLC18A2, SMC4, SPARC, SQLE, STRIP1, STX12, UNC45A, and XPC to the expression level of the housekeeping gene, thereby obtaining a normalized expression level of each of AAMP, CHTOP, EDC4, FYCO1, HNRNPU, HPN, KRT23, MAN2B2, MAX, MRPS25, NDUFS2, PPRC1, RAD23A, REPIN1, SDR39U1, SETBP1, SLC18A2, SMC4, SPARC, SQLE, STRIP1, STX12, UNC45A, and XPC;
(c) inputting each normalized expression level from step (b) into an algorithm to generate a score;
(d) comparing the score to a predetermined cutoff value; and
(e) determining that the prostate cancer is progressive when the score is greater than or equal to the predetermined cutoff value or determining that the prostate cancer is stable when the score is less than the predetermined cutoff value.

4. The method of claim 3, wherein the predetermined cutoff value is 50% on a scale of 0-100%.

5. A method of determining whether a prostate cancer in a subject has a low Gleason score (≤6) or a high Gleason score (≥7), the method comprising:
(a) determining the expression level of at least 24 biomarkers in a saliva sample from the subject, wherein the at least 24 biomarkers comprise AAMP, CHTOP, EDC4, FYCO1, HNRNPU, HPN, KRT23, MAN2B2, MAX, MRPS25, NDUFS2, PPRC1, RAD23A, REPIN1, SDR39U1, SETBP1, SLC18A2, SMC4, SPARC, SQLE, STRIP1, STX12, UNC45A, and XPC, and a housekeeping gene;
(b) normalizing the expression level of each of AAMP, CHTOP, EDC4, FYCO1, HNRNPU, HPN, KRT23, MAN2B2, MAX, MRPS25, NDUFS2, PPRC1, RAD23A, REPIN1, SDR39U1, SETBP1, SLC18A2, SMC4, SPARC, SQLE, STRIP1, STX12, UNC45A, and XPC to the expression level of the housekeeping gene, thereby obtaining a normalized expression level of each of AAMP, CHTOP, EDC4, FYCO1, HNRNPU, HPN, KRT23, MAN2B2, MAX, MRPS25, NDUFS2, PPRC1, RAD23A, REPIN1, SDR39U1, SETBP1, SLC18A2, SMC4, SPARC, SQLE, STRIP1, STX12, UNC45A, and XPC;
(c) inputting each normalized expression level from step (b) into an algorithm to generate a score;
(d) comparing the score to a predetermined cutoff value; and
(e) determining that the prostate cancer in the subject has a high Gleason score (≥7) when the score is greater than or equal to the predetermined cutoff value or determining that the prostate cancer in the subject has a low Gleason score (≤6) when the score is less than the predetermined cutoff value.

6. The method of claim 5, wherein the predetermined cutoff value is 50% on a scale of 0-100%.

7. A method of determining the completeness of surgery in a subject having a prostate cancer, the method comprising:
(a) determining the expression level of at least 24 biomarkers in a saliva sample from the subject after the surgery, wherein the 24 biomarkers comprise AAMP, CHTOP, EDC4, FYCO1, HNRNPU, HPN, KRT23, MAN2B2, MAX, MRPS25, NDUFS2, PPRC1, RAD23A, REPIN1, SDR39U1, SETBP1, SLC18A2, SMC4, SPARC, SQLE, STRIP1, STX12, UNC45A, XPC, and a housekeeping gene;
(b) normalizing the expression level of each of AAMP, CHTOP, EDC4, FYCO1, HNRNPU, HPN, KRT23, MAN2B2, MAX, MRPS25, NDUFS2, PPRC1, RAD23A, REPIN1, SDR39U1, SETBP1, SLC18A2, SMC4, SPARC, SQLE, STRIP1, STX12, UNC45A, and XPC to the expression level of the housekeeping gene, thereby obtaining a normalized expression level of each of AAMP, CHTOP, EDC4, FYCO1, HNRNPU, HPN, KRT23, MAN2B2, MAX, MRPS25, NDUFS2, PPRC1, RAD23A, REPIN1, SDR39U1, SETBP1, SLC18A2, SMC4, SPARC, SQLE, STRIP1, STX12, UNC45A, and XPC;
(c) inputting each normalized expression level from step (b) into an algorithm to generate a score;
(d) comparing the score to a predetermined cutoff value; and
(e) identifying that the prostate cancer is not completely removed when the score is greater than or equal to the score or identifying that the prostate cancer is completely removed when the score is less than the predetermined cutoff value.

8. The method of claim 7, wherein the predetermined cutoff value is 50% on a scale of 0-100%.

9. A method of evaluating the response of a subject having a prostate cancer to an anti-prostate cancer therapy, the method comprising:
(a) at a first time point:
(i) determining the expression level of at least 24 biomarkers in a saliva sample from the subject, wherein the at least 24 biomarkers comprise AAMP, CHTOP, EDC4, FYCO1, HNRNPU, HPN, KRT23, MAN2B2, MAX, MRPS25, NDUFS2, PPRC1, RAD23A, REPIN1, SDR39U1, SETBP1, SLC18A2, SMC4, SPARC, SQLE, STRIP1, STX12, UNC45A, XPC, and a housekeeping gene;
(ii) normalizing the expression level of each of AAMP, CHTOP, EDC4, FYCO1, HNRNPU, HPN, KRT23, MAN2B2, MAX, MRPS25, NDUFS2, PPRC1, RAD23A, REPIN1, SDR39U1, SETBP1, SLC18A2, SMC4, SPARC, SQLE, STRIP1, STX12, UNC45A, and XPC to the expression level of the housekeeping gene, thereby obtaining a normalized expression level of each of AAMP, CHTOP, EDC4, FYCO1, HNRNPU, HPN, KRT23, MAN2B2, MAX, MRPS25, NDUFS2, PPRC1, RAD23A, REPIN1, SDR39U1, SETBP1, SLC18A2, SMC4, SPARC, SQLE, STRIP1, STX12, UNC45A, and XPC; and
(iii) inputting each normalized expression level from step (a)(ii) into an algorithm to generate a first score;
(b) at a second time point, wherein the second time point is after the first time point and after the administration of the therapy to the subject:
(i) determining the expression level of the at least 24 biomarkers in a saliva sample from the subject;
(ii) normalizing the expression level of each of AAMP, CHTOP, EDC4, FYCO1, HNRNPU, HPN, KRT23, MAN2B2, MAX, MRPS25, NDUFS2, PPRC1, RAD23A, REPIN1, SDR39U1, SETBP1, SLC18A2, SMC4, SPARC, SQLE, STRIP1, STX12, UNC45A, and XPC to the expression level of the housekeeping gene, thereby obtaining a normalized expression level of each of AAMP, CHTOP, EDC4, FYCO1, HNRNPU, HPN, KRT23, MAN2B2, MAX, MRPS25, NDUFS2, PPRC1, RAD23A, REPIN1, SDR39U1, SETBP1, SLC18A2, SMC4, SPARC, SQLE, STRIP1, STX12, UNC45A, and XPC; and
(iii) inputting each normalized expression level from step (b)(ii) into an algorithm to generate a second score;
(c) comparing the first score and second score; and
(d) identifying that the subject is responsive to the anti-prostate cancer therapy when the second score is decreased as compared to the first score or identifying that the subject is not responsive to the anti-prostate cancer therapy when the second score is not decreased as compared to the normalized expression levels from step (a)(ii).

10. The method of claim 9, wherein the subject is identified as responsive to the anti-neuroendocrine cancer therapy when the second is at least 5% less than the first score.

11. The method of any one of the preceding claims, wherein the housekeeping gene is selected from the group consisting of ATG4B, RHOA, TOX4, TPT1, and TXNIP.

12. The method of claim 11, wherein the housekeeping gene is TOX4.

13. The method of any one of the preceding claims, having a sensitivity of at least 90%.

14. The method of any one of the preceding claims, having a specificity of at least 90%.

15. The method of any one of the preceding claims, wherein at least one of the at least 24 biomarkers is RNA, cDNA, or protein.

16. The method of claim 15, wherein when the biomarker is RNA, the RNA is reverse transcribed to produce cDNA, and the produced cDNA expression level is detected.

17. The method of any one of the preceding claims, wherein the expression level of the biomarker is detected by forming a complex between the biomarker and a labeled probe or primer.

18. The method of claim 15, wherein when the biomarker is protein, the protein detected by forming a complex between the protein and a labeled antibody.

19. The method of claim 18, wherein the label is a fluorescent label.

20. The method of claim 15, wherein when the biomarker is RNA or cDNA, the RNA or cDNA is detected by forming a complex between the RNA or cDNA and a labeled nucleic acid probe or primer.

21. The method of claim 20, wherein the label is a fluorescent label.

22. The method of claim 20 or claim 21, wherein the complex between the RNA or cDNA and the labeled nucleic acid probe or primer is a hybridization complex.

23. The method of any one of the preceding claims, wherein the first predetermined cutoff value is derived from a plurality of reference samples obtained from subjects not having or not diagnosed with a neoplastic disease.

24. The method of claim 23, wherein the neoplastic disease is prostate cancer.

25. The method of any one of the preceding claims wherein the algorithm is XGB, RF, glmnet, cforest, CART, treebag, knn, nnet, SVM-radial, SVM-linear, NB, or mlp.

26. The method of claim 25, wherein the algorithm is Random Forest.

27. The method of claim 26, wherein the machine learning algorithm is trained using the expression levels or normalized expression levels of the at least 24 biomarkers obtained from a plurality of reference samples obtained from subjects not having a neuroendocrine cancer and the expression levels or normalized expression levels of the at least 24 biomarkers from a plurality of reference samples obtained from subjects having neuroendocrine cancer.

28. The method of any one of the preceding claims, further comprising treating the subject identified as having prostate cancer with at least one anti-prostate cancer therapy.

29. The method of any one of the preceding claims, wherein the anti-prostate cancer therapy comprises comprise active surveillance, surgery, radiation therapy, cryotherapy, hormone therapy, chemotherapy, vaccine treatment, bone-directed treatment, or any combination thereof.

30. The method of claim 29, wherein the radiation therapy comprises external beam radiation, brachytherapy, radiopharmaceuticals or any combination thereof, preferably wherein the radiopharmaceuticals comprise 177Lu-PSMA.

31. The method of claim 29, wherein the hormone therapy comprises androgen suppression therapy.

32. The method of claim 29, wherein the chemotherapy comprises docetaxel, cabazitaxel, mitoxantrone, estramustine, or any combination thereof.

33. The method of claim 29, wherein the vaccine treatment comprises Sipuleucel-T.

34. The method of claim 29, wherein the bone-directed treatment comprises a bisphosphonate, denosumab, a corticosteroid, or a combination thereof.

35. The method of any one of the preceding claims, wherein the first time point is prior to the administration of the therapy to the subject.

36. The method of any one of the preceding claims, wherein the first time point is after the administration of the therapy to the subject.

37. The method of any one of the preceding claims, wherein the saliva sample is self-collected saliva into a container with stabilization fluid.
